(19) United States
(12) Patent Application Publication
(10) Pub. No.: US 2023/0220007 A1
(43) Pub. Date: Jul. 13, 2023

(54) SETD7 EPIGENETIC MODULATORS

(71) Applicant: Biorchestra Co., Ltd., Daejeon (KR)

(72) Inventor: Jin-Hyeob RYU, Daejeon (KR)

(73) Assignee: Biorchestra Co., Ltd., Daejeon (KR)

(21) Appl. No.: 17/996,330

(22) PCT Filed: Apr. 14, 2021
(86) PCT No.: PCT/IB2021/053091

§ 371 Date: Oct. 14, 2022

Related U.S. Application Data
(60) Provisional application No. 63/010,026, filed on Apr. 14, 2020.

(51) Int. Cl. C07K 7/08; C12N 15/86; A61K 47/18; A61K 47/22; A61P 3/10

(52) U.S. Cl. CPC C07K 7/08; C12N 15/86; A61K 47/183; A61K 47/22; A61P 3/10; C12N 2740/16043; C12N 2750/14143

(57) ABSTRACT

The present disclosure provides SETD7 modulators (e.g., polypeptides, polynucleotides, vectors, compositions, micelles, or pharmaceutical composition) which reduce or abolish SETD7 translocation to the nucleus, e.g., in response to stimuli such as increases in glucose levels. In some aspects, the SETD7 modulators mimic phosphorylated STED7, competing with STED7 and reducing or abolishing SETD7 nuclear translocation. In turn, the reduced SETD7 nuclear translocation results in a decrease in histone monomethylation. In some aspects, the SETD7 modulator is a catalytically inactive SETD7 protein. In some aspects, the SETD7 modulator is a polypeptide (e.g., a phosphomimetic polypeptide). In other aspects, the SETD7 modulator is a polynucleotide encoding a SETD7 polypeptide modulator, e.g., a phosphomimetic polypeptide or a mutant SETD7 protein. The SETD7 modulators of the present disclosure can be used to treat type diabetes or cancer.

### CROSS-REFERENCE TO RELATED APPLICATIONS

This PCT application claims the priority benefit of U.S. Provisional Application No. 63/010,026, filed on Apr. 14, 2020, which is herein incorporated by reference in its entirety.

### REFERENCE TO SEQUENCE LISTING SUBMITTED ELECTRONICALLY

The content of the electronically submitted sequence listing in ASCII text file (Name 4366_004PC01_Seqlisting_ST25; Size: 33,892 bytes; and Date of Creation: Apr. 13, 2021) filed with the application is incorporated herein by reference in its entirety.

### FIELD

The present disclosure provides compositions and methods for the treatment of diabetes and associated metabolic conditions or cancer using epigenetic modulators derived from histone-lysine N-methyltransferase SETD7.

### BACKGROUND

Epigenetics is broadly described as heritable changes in an organism caused by modifications of gene function that occur without a change in the genetic sequence. Epigenetic regulation of gene expression is a dynamic and reversible process that establishes normal cellular phenotypes but also contributes to human diseases such as type 2 diabetes and a wide array of vascular complications due to diabetes.

The epigenetic variations involved with diabetes can change chromatin structure as well as gene expression. Marpadga et al. (2012) Cardiovascular Research 90:421-29. Regardless of whether glycemic control has been achieved through treatment, these epigenetic mechanisms are lasting and do not change with the alteration of diet. Studies have shown that insulin resistance (IR), the hallmark of type 2 diabetes, may involve epigenetic control as a contributing factor. Studies also have shown that the islet dysfunction and development of diabetes in rats is associated with epigenetic silencing via DNA methylation of the gene Pdx1 promoter, which produces a key transcription factor that regulates beta-cell differentiation and insulin gene expression. Marpadga et al. (2012) Cardiovascular Research 90:421-29. Under high glucose conditions, islet-specific transcription factor Pdx1 has been shown to stimulate insulin expression by recruiting co-activators p300 and the Histone methyl transferase SETD7, which increased histone acetylation and H3K4me2, respectively, and the formation of open chromatin at the insulin promoter. In contrast, under low-glucose conditions, Pdx1 could recruit co-repressors HDAC1/2, which led to inhibition of insulin gene expression. Furthermore, Pdx1 also mediated β-cell-specific expression of SET7/9, which may regulate genes involved in glucose-induced insulin secretion.

Accordingly, there is a need for epigenetic modulators that can be used for the treatment of diseases and conditions in which epigenetic mechanisms are causative or contributing, including metabolic disorders such as diabetes and cancer.

### BRIEF SUMMARY

The present disclosure provides epigenetic modulators, in particular, SETD7 modulators, that can be used for the treatment of diseases and conditions such as type 2 diabetes or cancer. In some aspects, the present disclosure provides an isolated polynucleotide encoding a polypeptide which comprises the sequence X₁VAYGYDHX₂PPGKX₃ (SEQ ID NO: 1), wherein each of X₁, X₂, and X₃ is (i) Serine (S) or Threonine (T); (ii) a phosphomimetic amino acid or analog thereof; or, (iii) a combination thereof, wherein the polypeptide sequence is not TVAYGYDHSPPGKS (SEQ ID NO: 2; wild type), wherein one, two, three, four or five amino acids other than X₁, X₂, and X₃ are optionally substituted with respect to their corresponding amino acids in SEQ ID NO: 2, and wherein the polypeptide is capable of modulating nuclear translocation of endogenous SET domain containing 7, histone lysine methyltransferase (SETD7). The present disclosure also provides an isolated polypeptide (e.g., a synthetic polypeptide) which comprises the sequence X₁VAYGYDHX₂PPGKX₃ (SEQ ID NO: 1), wherein each of X₁, X₂, and X₃ is (i) Serine (S) or Threonine (T); (ii) a phosphomimetic amino acid or analog thereof; or, (iii) a combination thereof, wherein the polypeptide sequence is not TVAYGYDHSPPGKS (SEQ ID NO: 2; wild type), wherein one, two, three, four or five amino acids other than X₁, X₂, and X₃ are optionally substituted with respect to their corresponding amino acids in SEQ ID NO: 2, and wherein the polypeptide is capable of modulating nuclear translocation of endogenous SET domain containing 7, histone lysine methyltransferase (SETD7).

In some aspects, the phosphomimetic amino acid is aspartic acid (D) or glutamic acid (E). In some aspects, the phosphomimetic amino acid is phosphoserine or phosphothreonine. In some aspects, the phosphomimetic amino acid analog is a non-cleavable analog. In some aspects, the non-cleavable analog is a phosphoserine non-hydrolyzable analog. In some aspects, the non-hydrolyzable analog of phosphoserine is 2-amino-4-phosphobutyric acid. In some aspects, the optionally substituted amino acids are conservative amino acid substitutions. In some aspects, at least one tyrosine (Y) is substituted. In some aspects, at least one tyrosine is substituted with phosphotyrosine, aspartic acid, glutamic acid, or an analog thereof. In some aspects, the phosphotyrosine analog is a non-hydrolyzable analog.

In some aspects, the non-hydrolyzable analog of phosphotyrosine is 4-phosphomethyl-L-phenylalanine (Pmp). In some aspects, the phosphomimetic amino acid analog is a thiophosphate analog. In some aspects, the thiophosphate analog is thiophosphoserine.

In some aspects, the sequence of the polypeptide comprises a sequence selected from the group consisting of SEQ ID NOS: 3, 4, 5, 6, 7, 8, 9, 10, 11, 12, 13, 14, 15, 16, 17 and 18. In some aspects, a polypeptide set forth in SEQ ID NO:3 (x-TVAYGYDHSPPGKS-y) comprises N- and/or C-terminal modification, wherein x is an N-terminal modification and y is a C-terminal modification. In some aspects, the N-terminal modification is acetylation. In some aspects, the C-terminal modification is amidation.

In some aspects, the polypeptide comprises the sequence

(SEQ ID NO: 19)
X₄VAX₅GX₆DHX₇PPGKX₈

wherein X₄, X₇ and X₈ are selected from the group consisting of serine, threonine, aspartic acid, glutamic acid, 2-amino-4-phosphobutyric acid, and thiophosphoserine; and wherein X₅ and X₆ are selected from the group consisting of tyrosine, and 4-phosphomethyl-L-phenylalanine. In some aspects, the polypeptide further comprises (i) an L, EL, EEL, DEEL (SEQ ID NO:20), or ADEEL (SEQ ID:21) amino acid sequence appended to its N-terminus; (ii) a G, GP, GPE, GPEA (SEQ ID NO:22), or GPEAP (SEQ ID NO:23) amino acid sequence appended to its C-terminus; or, (iii) a combination thereof.

In some aspects, the polypeptide comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence as set forth in SEQ ID NOS: 24, 25, 26, 27, 28, 29, 30 or 31.

In some aspects, the polypeptide consists of or consists essentially of the sequence as set forth in SEQ ID NOS: 24, 25, 26, 27, 28, 29, 30 or 31.

The present disclosure also provides an isolated polynucleotide encoding a polypeptide which consists of or consists essentially of the sequence as set forth in SEQ ID NO: 2 and (i) an L, EL, EEL, DEEL (SEQ ID NO:20), or ADEEL (SEQ ID:21) amino acid sequence appended to its N-terminus; (ii) a G, GP, GPE, GPEA (SEQ ID NO:22), or GPEAP (SEQ ID NO:23) amino acid sequence appended to its C-terminus; or, (iii) a combination thereof.

In some aspects, the polypeptide consists essentially of or consists of an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence as set forth in ADEELTVAYGYDHSPPGKSGPEAP (SEQ ID NO:32). In some aspects, the polypeptide consists or consists essentially of ADEELTVAYGYDHSPPGKSGPEAP (SEQ ID NO:32).

In some aspects, the polypeptide has at least 14 amino acids, at least 15 amino acids, at least 16 amino acids, at least 17 amino acids, at least 18 amino acids, at least 19 amino acids, at least 20 amino acids, at least 21 amino acids, at least 22 amino acids, at least 23 amino acids, or at least 24 amino acids in length. In some aspects, the polypeptide comprises an N-terminal capping modification, a C-terminal capping modification, or a combination thereof. In some aspects, the N-terminal capping modification is an N-terminal acetylation, formylation, acylation, pyroglutamylation, or carbamate, sulfonamide, or alkylamine modification. In some aspects, the C-terminal capping modification is a C-terminal amidation, N-alkyl amidation, aldehyde modification, or esterification.

In some aspects, the isolated polynucleotide comprises a nucleic acid sequence at least about 70%, at least about 75%, at least about 80%, at least about 85%, at least about 90%, at least about 95%, at least about 96%, at least about 97%, at least about 98%, at least about 99%, or about 100% sequence identity to the sequence as set forth in 5′-GCNGAYGARGARYUNGARGUNGCNUAYGGNUAYGAYCAYGAYCCNCCNGGNAAR GAYGGNCCNGARGCNCCNURR-3′ (SEQ ID NO:33), wherein N is any nucleotide (A, G, T, or C), Y is a pyrimidine (C or T), R is a purine (A or G). In some aspects, the isolated polynucleotide comprises a nucleic acid sequence as set forth in 5′-GCNGAYGARGARYUNGARGUNGCNUAYGGNUAYGAYCAYGAYCCNCCNGGNAAR GAYGGNCCNGARGCNCCNURR-3′ (SEQ ID NO:33), wherein N is any nucleotide (A, G, T, or C), Y is a pyrimidine (C or T), R is a purine (A or G).

In some aspects, the isolated polynucleotide consists of or consists essentially of a nucleic acid sequence at least about 70%, at least about 75%, at least about 80%, at least about 85%, at least about 90%, at least about 95%, at least about 96%, at least about 97%, at least about 98%, at least about 99%, or about 100% sequence identity to the sequence as set forth in 5′-GCNGAYGARGARYUNACNGUNGCNUAYGGNUAYGAYCAYWSNCCNCCNGGNA ARWSNGGNCCNGARGCNCCNURR-3′ (SEQ ID NO: 34).

In some aspects, the polypeptide comprises at least about 20 amino acids, at least about 30 amino acids, at least about 40 amino acids, at least about 50 amino acids, at least about 60 amino acids, at least about 70 amino acids, at least about 80 amino acids, at least about 90 amino acids, at least about 100 amino acids, at least about 120 amino acids, at least about 140 amino acids, at least about 160 amino acids, at least about 180 amino acids, at least about 200 amino acids, at least about 220 amino acids, at least about 240 amino acids, at least about 260 amino acids, at least about 280 amino acids, at least about 300 amino acids, at least about 320 amino acids, at least about 340 amino acids or at least about 360 amino acids in length.

In some aspects, the polypeptide is enzymatically inactive.

In some aspects, the polypeptide comprises at least about 70%, at least about 75%, at least about 80%, at least about 85%, at least about 90%, at least about 95%, at least about 96%, at least about 97%, at least about 98%, at least about 99%, or about 100% sequence identity to the sequence as set forth in SEQ ID NO: 35. In some aspects, the polypeptide consists of or consists essentially of the sequence as set forth in SEQ ID NO: 35. In some aspects, the isolated polynucleotide comprises or consists of the sequence set forth in SEQ ID NO: 36.

In some aspects, the polypeptide further comprises at least one heterologous moiety. In some aspects, at least one heterologous moiety comprises a serum half-life extending moiety. In some aspects, the serum half-life extending moiety comprises an Fc region, albumin, albumin binding polypeptide, a fatty acid, PAS, the β subunit of the C-terminal peptide (CTP) of human chorionic gonadotropin, polyethylene glycol (PEG), hydroxyethyl starch (HES), XTEN, albumin-binding small molecules, or a combination thereof.

In some aspects, the serum half-life of the polypeptide is at least about 10%, at least about 20%, at least about 30%, at least about 40%, at least about 50%, at least about 60%, at least about 70%, at least about 80%, at least about 90%, or at least about 100% higher than the plasma half-life of a corresponding polypeptide without a serum half-life extending moiety. In some aspects, the at least one heterologous moiety comprises a detectable moiety.

In some aspects, the polynucleotide is a DNA or an RNA. In some aspects, the polynucleotide comprises at least one chemically modified nucleobase, sugar, backbone, or any combination thereof. In some aspects, the polynucleotide is codon optimized.

The present disclosure also provides a vector comprising an isolated polynucleotide disclosed herein. In some aspects, the vector is a viral vector. In some aspects, the viral vector is an adenoviral vector or an adenoassociated viral vector. In some aspects, the adenoviral vector is a third generation adenoviral vector. In some aspects, the viral vector is a retroviral vector. In some aspects, the retroviral vector is a lentiviral vector. In some aspects, the lentiviral vector is a third or fourth generation lentiviral vector. Also provided is a polypeptide encoded by any of the polynucleotides disclosed herein.

Also provided is a composition comprising a polynucleotide, polypeptide, or vector disclosed herein, and a delivery agent. In some aspects, the delivery agent comprises a lipidoid, a liposome, a lipoplex, a lipid nanoparticle, a polymeric compound, a peptide, a protein, a cell, a nanoparticle mimic, a nanotube, or a conjugate. In some aspects, the delivery agent comprises a cationic carrier unit comprising

[WP]-L1-[CC]-L2-[AM] (formula I)

or

[WP]-L1-[AM]-L2-[CC] (formula II)

wherein
WP is a water-soluble biopolymer moiety;
CC is a positively charged carrier moiety;
AM is an optional adjuvant moiety; and,
L1 and L2 are independently optional linkers, and
wherein when mixed with a nucleic acid at an ionic ratio of about 1:1, the cationic carrier unit forms a micelle.

In some aspects, a polynucleotide, vector, or polypeptide disclosed herein interacts with the cationic carrier unit via an ionic bond.

In some aspects, the water-soluble polymer comprises a poly(alkylene glycol), a poly(oxyethylated polyol), a poly(olefinic alcohol), a poly(vinylpyrrolidone), a poly(hydroxyalkylmethacrylamide), apoly(hydroxyalkylmethacrylate), a poly(saccharide), a poly(α-hydroxy acid), a poly(vinyl alcohol), a polyglycerol, a polyphosphazene, a polyoxazoline (“POZ”) poly(N-acryloylmorpholine), or any combinations thereof. In some aspects, the water-soluble polymer comprises polyethylene glycol (“PEG”), polyglycerol, or poly(propylene glycol) (“PPG”). In some aspects, the water-soluble polymer comprises:

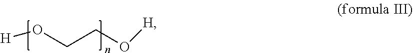

wherein n is 1-1000.

In some aspects, n is at least about 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, or at least about 141. In some aspects, n is about 80 to about 90, about 90 to about 100, about 100 to about 110, about 110 to about 120, about 120 to about 130, about 140 to about 150, about 150 to about 160.

In some aspects, the water-soluble polymer is linear, branched, or dendritic. In some aspects, the cationic carrier moiety comprises one or more basic amino acids. In some aspects, the cationic carrier moiety comprises at least three, at least four, at least five, at least six, at least seven, at least eight, at least nine, at least ten, at least 11, at least 12, at least 13, at least 14, at last 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 basic amino acids. In some aspects, the cationic carrier moiety comprises about 30 to about 50 basic amino acids. In some aspects, the basic amino acid comprises arginine, lysine, histidine, or any combination thereof. In some aspects, the cationic carrier moiety comprises about 40 lysine monomers.

In some aspects, the optional adjuvant moiety is capable of modulating an immune response, an inflammatory response, and/or a tissue microenvironment. In some aspects, the optional adjuvant moiety comprises an imidazole derivative, an amino acid, a vitamin, or any combination thereof. In some aspects, the optional adjuvant moiety comprises:

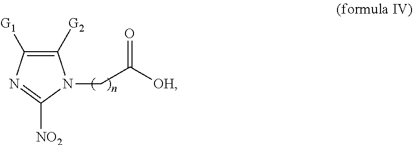

wherein each of G1 and G2 is H, an aromatic ring, or 1-10 alkyl, or G1 and G2 together form an aromatic ring, and wherein n is 1-10.

In some aspects, the optional adjuvant moiety comprises nitroimidazole. In some aspects, the optional adjuvant moiety comprises metronidazole, tinidazole, nimorazole, dimetridazole, pretomanid, ornidazole, megazol, azanidazole, benznidazole, or any combination thereof. In some aspects, the optional adjuvant moiety comprises an amino acid.

In some aspects, the optional adjuvant moiety comprises

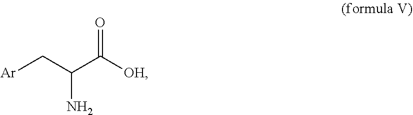

wherein Ar is

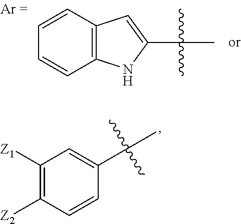

and
wherein each of Z1 and Z2 is H or OH.

In some aspects, the optional adjuvant moiety comprises a vitamin. In some aspects, the vitamin comprises a cyclic ring or cyclic hetero atom ring and a carboxyl group or hydroxyl group. In some aspects, the vitamin comprises:

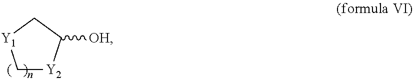

wherein each of Y1 and Y2 is C, N, O, or S, and wherein n is 1 or 2.

In some aspects, the vitamin is selected from the group consisting of vitamin A, vitamin B1, vitamin B2, vitamin B3, vitamin B6, vitamin B7, vitamin B9, vitamin B12, vitamin C, vitamin D2, vitamin D3, vitamin E, vitamin M, vitamin H, and any combination thereof. In some aspects, the vitamin is vitamin B3.

In some aspects, the optional adjuvant moiety comprises at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, or at least about 20 vitamin B3 units. In some aspects, the optional adjuvant moiety comprises about 10 vitamin B3 units.

In some aspects, the composition comprises about a water-soluble biopolymer moiety with about 120 to about 130 PEG units, a cationic carrier moiety comprising a poly-lysine with about 30 to about 40 lysines, and an adjuvant moiety with about 5 to about 10 vitamin B3.

The present disclosure also provides a micelle comprising a composition disclosed herein wherein a polynucleotide, vector, or polypeptide disclosed herein, and the delivery agent are associated with each other. In some aspects, the association is a covalent bond, a non-covalent bond, or an ionic bond. In some aspects, the positive charge of the cationic carrier moiety of the cationic carrier unit is sufficient to form a micelle when mixed with the polynucleotide, vector, or polypeptide disclosed herein in a solution, wherein the overall ionic ratio of the positive charges of the cationic carrier moiety of the cationic carrier unit and the negative charges of the polynucleotide, vector, or polypeptide in the solution is about 1:1.

In some aspects, the cationic carrier unit is capable of protecting the polynucleotide, vector, or polypeptide from enzymatic degradation.

The present disclosure also provides a cell comprising a polynucleotide or vector disclosed herein. The present disclosure also provides a pharmaceutical composition comprising a polynucleotide, vector, composition, micelle, or cell disclosed herein, and an excipient. Also provided is a method for preventing or reducing nuclear translocation of SETD7 in a cell comprising contacting the cell with an effective amount of a polynucleotide, vector, composition, micelle, or pharmaceutical composition disclosed herein.

Also provided is a method for preventing or reducing nuclear accumulation of SETD7 in a cell comprising contacting the cell with an effective amount of a polynucleotide, vector, composition, micelle, or pharmaceutical composition disclosed herein. The present disclosure also provides a method for preventing or reducing histone H3K4 monomethylation in a cell comprising contacting the cell with an effective amount of a polynucleotide, vector, composition, micelle, or pharmaceutical composition disclosed herein. Also provided is a method for preventing or reducing p53 monomethylation in a cell comprising contacting the cell with an effective amount of a polynucleotide, vector, composition, micelle, or pharmaceutical composition disclosed herein.

Also provided is a method for treating a metabolic disease or disorder in a subject in need thereof comprising administering to the subject a therapeutically effective amount of a polynucleotide, vector, composition, micelle, or pharmaceutical composition disclosed herein. In some aspects, the metabolic disease or disorder is diabetes, obesity, or insulin resistance. In some aspects, the diabetes is type 2 diabetes mellitus.

The present disclosure also provides a method to treat a cancer in a subject in need thereof comprising administering to the subject a therapeutically effective amount of a polynucleotide, vector, composition, micelle, or pharmaceutical composition disclosed herein.

The present disclosure also provides a kit comprising a polynucleotide, vector, composition, micelle, or pharmaceutical composition disclosed herein. Also provided is an article of manufacture comprising a polynucleotide, vector, composition, micelle, or pharmaceutical composition disclosed herein. In some aspects, the kit or article of manufacture, further comprises instructions for use according to the methods disclosed herein.

The present disclosure also provides a polynucleotide, vector, composition, micelle, or pharmaceutical composition disclosed herein for use as a medicament. Also provided is a polynucleotide, vector, composition, micelle, or pharmaceutical composition disclosed herein for use in the treatment of a metabolic disease in a subject in need thereof. In some aspects of the polynucleotide, vector, composition, micelle, cell, or pharmaceutical composition for use disclosed herein, the metabolic disease is diabetes.

The present disclosure also provides the use of a polynucleotide, vector, composition, micelle, or pharmaceutical composition disclosed herein. in the manufacture of treatment for a metabolic disease. In some aspects, the metabolic disease in diabetes.

### BRIEF DESCRIPTION OF THE DRAWINGS/FIGURES

FIG. 1 is a schematic representation of the domain structure of SETD7 and the location of phosphorylatable sites in its C-terminal regions.

FIG. 2 is a schematic representation showing a experimental design to identify the SET7D phosphorylated amino acids responsible for its nuclear translocation.

FIG. 3 shows SETD7 cellular distribution in response to changes in glucose levels for wild type SETD7 (WT) and five substitutions (S225A, Y305A, T332A, S340A, and S345A) in which amino acid susceptible to phosphorylation are replaced with alanine. When amino acids Thr332, Ser340, or Ser345 cannot be phosphorylated, and SET7D is not translocated to the nucleus in response to changes in glucose.

FIG. 4 shows SETD7 cellular distribution in response to changes in glucose levels for wild type SETD7 (WT) and four substitutions or combinations thereof, namely, T332A, S340A, T332E, S340D, T332E/S340D/S345D, and H297A/T332E/S340D/S345D. When amino acids Thr332 or Ser340 cannot be phosphorylated and SETD7 is not translocated to the nucleus in response to changes in glucose. SETD7 is translocated when Thr332, Ser340, and Ser345 are replaced with glutamic or aspartic acid (phosphomimetic substitutions).

### DETAILED DESCRIPTION

The present disclosure is directed to epigenetic modulators related to histone-lysine N-methyltransferase SETD7 (“SETD7”). In some aspects, these SETD7 modulators can be used to treat or prevent metabolic disorders, e.g., diabetes and its complications (as descried, e.g, in the background section), and related conditions such as nonalcoholic fatty liver disease (NALFD) or obesity. In other aspects, the SETD7 modulators disclosed herein can be used to treat cancer. Some of the modulators disclosed herein are phosphorylatable or phosphomimetic peptides (e.g., synthetic peptides or recombinantly expressed peptides) that compete with SETD7 for the same transporters, and therefore prevent or reduce the nuclear translocation of SETD7. In other aspects, the modulators are inactive forms of SETD7 which would be translocated to the nucleus like SETD7, but would not be able to methylate histones and/or other nuclear proteins.

In some aspects, the SETD7 modulators are antibodies or aptamers that bind to SETD7 (to either phosphorylated or non-phosphorylated forms of SETD7), or antisense oligonucleotides capable of binding to mRNAs encoding SETD7.

The present disclosure also provides, e.g., pharmaceutical compositions, cells, micelles, kits or articles of manufacture comprising at least one SETD7 modulator of the present disclosure.

Before the present disclosure is described in greater detail, it is to be understood that this disclosure is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order which is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, ‘a’ represents adenine, ‘c’ represents cytosine, ‘g’ represents guanine, ‘t’ represents thymine, and ‘u’ represents uracil.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The terms “administration,” “administering,” and grammatical variants thereof refer to introducing a composition, such as a SETD7 modulator of the present disclosure, into a subject via a pharmaceutically acceptable route. The introduction of a composition, such as a micelle comprising a SETD7 modulator of the present disclosure, into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least 70% identical, at least 80% identical, at least 90% identical, or at least 95% identical to one another. In some aspects, two or more sequences are said to be “highly conserved” if they are about 70% identical, about 80% identical, about 90% identical, about 95%, about 98%, or about 99% identical to one another. In some aspects, two or more sequences are said to be “conserved” if they are at least 30% identical, at least 40% identical, at least 50% identical, at least 60% identical, at least 70% identical, at least 80% identical, at least 90% identical, or at least 95% identical to one another. In some aspects, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an polynucleotide or polypeptide or may apply to a portion, region or feature thereof.

The term “derived from,” as used herein, refers to a component that is isolated from or made using a specified molecule or organism, or information (e.g., amino acid or nucleic acid sequence) from the specified molecule or organism. For example, a nucleic acid sequence that is derived from a second nucleic acid sequence can include a nucleotide sequence that is identical or substantially similar to the nucleotide sequence of the second nucleic acid sequence. In the case of nucleotides or polypeptides, the derived species can be obtained by, for example, naturally occurring mutagenesis, artificial directed mutagenesis or artificial random mutagenesis. The mutagenesis used to derive nucleotides or polypeptides can be intentionally directed or intentionally random, or a mixture of each. The mutagenesis of a nucleotide or polypeptide to create a different nucleotide or polypeptide derived from the first can be a random event (e.g., caused by polymerase infidelity) and the identification of the derived nucleotide or polypeptide can be made by appropriate screening methods, e.g., as discussed herein. Mutagenesis of a polypeptide typically entails manipulation of the polynucleotide that encodes the polypeptide. In some aspects, a nucleotide or amino acid sequence that is derived from a second nucleotide or amino acid sequence has a sequence identity of at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% to the second nucleotide or amino acid sequence, respectively, wherein the first nucleotide or amino acid sequence retains the biological activity of the second nucleotide or amino acid sequence.

The terms “complementary” and “complementarity” refer to two or more oligomers (i.e., each comprising a nucleobase sequence), or between an oligomer and a target gene, that are related with one another by Watson-Crick base-pairing rules. For example, the nucleobase sequence “T-G-A (5′→3′),” is complementary to the nucleobase sequence “A-C-T (3′→5′).” Complementarity may be “partial,” in which less than all of the nucleobases of a given nucleobase sequence are matched to the other nucleobase sequence according to base pairing rules. For example, in some aspects, complementarity between a given nucleobase sequence and the other nucleobase sequence may be about 70%, about 75%, about 80%, about 85%, about 90% or about 95%. Or, there may be “complete” or “perfect” (100%) complementarity between a given nucleobase sequence and the other nucleobase sequence to continue the example. The degree of complementarity between nucleobase sequences has significant effects on the efficiency and strength of hybridization between the sequences.

The term “downstream” refers to a nucleotide sequence that is located 3′ to a reference nucleotide sequence. In certain aspects, downstream nucleotide sequences relate to sequences that follow the starting point of transcription. For example, the translation initiation codon of a gene is located downstream of the start site of transcription.

The terms “excipient” and “carrier” are used interchangeably and refer to an inert substance added to a pharmaceutical composition to further facilitate administration of a compound, e.g., a SETD7 modulator of the present disclosure.

As used herein, the term “homology” refers to the overall relatedness between polymeric molecules, e.g. between nucleic acid molecules (e.g. DNA molecules and/or RNA molecules) and/or between polypeptide molecules. Generally, the term “homology” implies an evolutionary relationship between two molecules. Thus, two molecules that are homologous will have a common evolutionary ancestor. In the context of the present disclosure, the term homology encompasses both to identity and similarity.

In some aspects, polymeric molecules are considered to be “homologous” to one another if at least 25%, 30%, 35%, 40%, 45%, 50%, 55%, 60%, 65%, 70%, 75%, 80%, 85%, 90%, 95%, or 99% of the monomers in the molecule are identical (exactly the same monomer) or are similar (conservative substitutions). The term “homologous” necessarily refers to a comparison between at least two sequences (polynucleotide or polypeptide sequences).

As used herein, the term “identity” refers to the overall monomer conservation between polymeric molecules, e.g., between polypeptide molecules or polynucleotide molecules (e.g. DNA molecules and/or RNA molecules). The term “identical” without any additional qualifiers, e.g., protein A is identical to protein B, implies the sequences are 100% identical (100% sequence identity). Describing two sequences as, e.g., “70% identical,” is equivalent to describing them as having, e.g., “70% sequence identity.”

Calculation of the percent identity of two polypeptide or polynucleotide sequences, for example, can be performed by aligning the two sequences for optimal comparison purposes (e.g., gaps can be introduced in one or both of a first and a second polypeptide or polynucleotide sequences for optimal alignment and non-identical sequences can be disregarded for comparison purposes). In certain aspects, the length of a sequence aligned for comparison purposes is at least 30%, at least 40%, at least 50%, at least 60%, at least 70%, at least 80%, at least 90%, at least 95%, or 100% of the length of the reference sequence. The amino acids at corresponding amino acid positions, or bases in the case of polynucleotides, are then compared.

When a position in the first sequence is occupied by the same amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

As used herein, the terms “isolated,” “purified,” “extracted,” and grammatical variants thereof are used interchangeably and refer to the state of a preparation of desired composition of the present disclosure, e.g., a SETD7 modulator of the present disclosure, that has undergone one or more processes of purification. In some aspects, isolating or purifying as used herein is the process of removing, partially removing (e.g., a fraction) of a composition of the present disclosure, e.g., a SETD7 modulator of the present disclosure from a sample containing contaminants.

In some aspects, an isolated composition has no detectable undesired activity or, alternatively, the level or amount of the undesired activity is at or below an acceptable level or amount. In other aspects, an isolated composition has an amount and/or concentration of desired composition of the present disclosure, at or above an acceptable amount and/or concentration and/or activity. In other aspects, the isolated composition is enriched as compared to the starting material from which the composition is obtained. This enrichment can be by at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.9%, at least about 99.99%, at least about 99.999%, at least about 99.9999%, or greater than 99.9999% as compared to the starting material.

In some aspects, isolated preparations are substantially free of residual biological products. In some aspects, the isolated preparations are 100% free, at least about 99% free, at least about 98% free, at least about 97% free, at least about 96% free, at least about 95% free, at least about 94% free, at least about 93% free, at least about 92% free, at least about 91% free, or at least about 90% free of any contaminating biological matter. Residual biological products can include abiotic materials (including chemicals) or unwanted nucleic acids, proteins, lipids, or metabolites.

The term “linked” as used herein refers to a first amino acid sequence or polynucleotide sequence covalently or non-covalently joined to a second amino acid sequence or polynucleotide sequence, respectively. The first amino acid or polynucleotide sequence can be directly joined or juxtaposed to the second amino acid or polynucleotide sequence or alternatively an intervening sequence can covalently join the first sequence to the second sequence. The term “linked” means not only a fusion of a first polynucleotide sequence to a second polynucleotide sequence at the 5′-end or the 3′-end, but also includes insertion of the whole first polynucleotide sequence (or the second polynucleotide sequence) into any two nucleotides in the second polynucleotide sequence (or the first polynucleotide sequence, respectively). The first polynucleotide sequence can be linked to a second polynucleotide sequence by a phosphodiester bond or a linker. The linker can be, e.g., a polynucleotide.

In some aspects, a SETD7 modulator of the present disclosure, wherein the modulator is a polynucleotide, can be linked to nucleic acid sequence encoding a heterologous moiety (e.g., a half life extending moiety). In some aspects, a SETD7 modulator of the present disclosure, wherein the modulator is a polypeptide, a heterologous moiety (e.g., a half life extending moiety) can be linked to the modulator, for example, via genetic fusion (i.e., the modulators and the heterologous moiety are recombinantly expressed as a single polypeptide chain, i.e., a fusion protein), via conjugation (i.e., the heterologous moiety and the modulator are chemically conjugated), or via synthesis (i.e., the modulator and the heterologous can be synthesized as a single molecule, e.g., via solid phase peptide synthesis or solid phase polynucleotide synthesis).

As used herein, the terms “modulate,” “modify,” and grammatical variants thereof, generally refer when applied to a specific concentration, level, expression, function or behavior, to the ability to alter, by increasing or decreasing, e.g., directly or indirectly promoting/stimulating/up-regulating or interfering with/inhibiting/down-regulating the specific concentration, level, expression, function or behavior, such as, e.g., to act as an antagonist or agonist. In some instances a modulator can increase and/or decrease a certain concentration, level, activity or function relative to a control, or relative to the average level of activity that would generally be expected or relative to a control level of activity. In some aspects of the present disclosure a modulator, e.g., a SETD7 modulator, can modulate (e.g., decrease, alter, or abolish) SETD7 methyltransferase activate, modulate SETD7 distribution in cytoplasm and nucleus (e.g., decrease, alter, or abolish SETD7 nuclear levels), modulate SETD7 nuclear translocation (e.g., decrease, alter, or abolish nuclear translocation, modulate SETD7 methyltransferase substrate specificity (e.g., change preferred methylation target), modulate SETD7 methyltransferase activity (e.g., monomethyltransferase activity versus dimethyltransferase activity), or any combination thereof.

“Nucleic acid,” “nucleic acid molecule,” “nucleotide sequence,” “polynucleotide,” and grammatical variants thereof are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein.

The terms “pharmaceutically acceptable carrier,” “pharmaceutically acceptable excipient,” and grammatical variations thereof, encompass any of the agents approved by a regulatory agency of the U.S. Federal government or listed in the U.S. Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

As used herein, the term “pharmaceutical composition” refers to one or more of the compounds described herein, such as, e.g., a SETD7 modulator of the present disclosure, mixed or intermingled with, or suspended in one or more other chemical components, such as pharmaceutically acceptable carriers and excipients. One purpose of a pharmaceutical composition is to facilitate administration of preparations comprising a SETD7 modulator of the present disclosure to a subject.

The term “polynucleotide” as used herein refers to polymers of nucleotides of any length, including ribonucleotides, deoxyribonucleotides, analogs thereof, or mixtures thereof.

In some aspects of the present disclosure, a SETD7 modulator can be a polynucleotide SETD7 modulator encoding a SETD7 polypeptide modulator, wherein the SETD7 polypeptide modulator can compete or bind to endogenous SETD7. In other aspects, a SETD7 modulator be can a polynucleotide SETD7 modulator interacting with a nucleic acid encoding endogenous SETD7 (e.g., an antisense oligonucleotide SETD7 modulator targeting mRNA encoding endogenous SETD7). In some aspects, a SETD7 modulator can be a polynucleotide encoding a catalytically inactive form of SETD7, e.g., a SETD7 protein lacking monomethyl transferase enzymatic activity.

In some aspects, the term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide.

More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA.

In some aspects of the present disclosure a polynucleotide can be, e.g., an oligonucleotide, such as an antisense oligonucleotide or an oligonucleotide encoding a peptide. In some aspects, the oligonucleotide is an RNA. In some aspects, the RNA is a synthetic RNA. In some aspects, the synthetic RNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine).

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. The term “polypeptide,” as used herein, refers to proteins, polypeptides, and peptides of any size, structure, or function.

Polypeptides include gene products, naturally occurring polypeptides, synthetic polypeptides, homologs, orthologs, paralogs, fragments and other equivalents, variants, and analogs of the foregoing.

A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to 50 amino acids long, e.g., about 5, 10, 15, 20, 25, 30, 35, 40, 45, or 50 amino acids long.

The terms “prevent,” “preventing,” and variants thereof as used herein, refer partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition. In some aspects, preventing an outcome is achieved through prophylactic treatment.

As used herein, “prophylactic” refers to a therapeutic or course of action used to prevent the onset of a disease or condition, or to prevent or delay a symptom associated with a disease or condition.

As used herein, a “prophylaxis” refers to a measure taken to maintain health and prevent the onset of a disease or condition, or to prevent or delay a symptom associated with a disease or condition.

As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art. It is understood that percentage of similarity is contingent on the comparison scale used, i.e., whether the amino acids are compared, e.g., according to their evolutionary proximity, charge, volume, flexibility, polarity, hydrophobicity, aromaticity, isoelectric point, antigenicity, or combinations thereof.

The terms “subject,” “patient,” “individual,” and “host,” and variants thereof are used interchangeably herein and refer to any mammalian subject, including without limitation, humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired, particularly humans. The methods described herein are applicable to both human therapy and veterinary applications.

As used herein, the phrase “subject in need thereof” includes subjects, such as mammalian subjects, that would benefit from administration of a micelle of the disclosure, e.g., to improve hemostasis.

As used herein the term “therapeutically effective amount” is the amount of reagent or pharmaceutical compound comprising a SETD7 modulator of the present disclosure that is sufficient to a produce a desired therapeutic effect, pharmacologic and/or physiologic effect on a subject in need thereof. A therapeutically effective amount can be a “prophylactically effective amount” as prophylaxis can be considered therapy.

The terms “treat,” “treatment,” or “treating,” as used herein refers to, e.g., the reduction in severity of a disease or condition; the reduction in the duration of a disease course; the amelioration or elimination of one or more symptoms associated with a disease or condition (e.g., diabetes); the provision of beneficial effects to a subject with a disease or condition, without necessarily curing the disease or condition. The term also include prophylaxis or prevention of a disease or condition or its symptoms thereof.

The term “upstream” refers to a nucleotide sequence that is located 5′ to a reference nucleotide sequence.

The term “amino acid substitution” refers to replacing an amino acid residue present in a parent or reference sequence (e.g., a wild type sequence) with another amino acid residue. An amino acid can be substituted in a parent or reference sequence (e.g., a wild type polypeptide sequence), for example, via chemical peptide synthesis or through recombinant methods known in the art. Accordingly, a reference to a “substitution at position X” refers to the substitution of an amino acid present at position X with an alternative amino acid residue. In some aspects, substitution patterns can be described according to the schema AnY, wherein A is the single letter code corresponding to the amino acid naturally or originally present at position n, and Y is the substituting amino acid residue. In other aspects, substitution patterns can be described according to the schema An(YZ), wherein A is the single letter code corresponding to the amino acid residue substituting the amino acid naturally or originally present at position n, and Y and Z are alternative substituting amino acid residues that can replace A.

In some aspects of the present disclosure, substitutions (even when they are referred to as amino acid substitution) can be conducted at the nucleic acid level, i.e., substituting an amino acid residue with an alternative amino acid residue can be conducted by substituting the codon encoding the first amino acid with a codon encoding the second amino acid. In other aspects, substitutions can be conducted at protein level, e.g., substituting an amino acid residues with an alternative amino acid residue during chemical synthesis (e.g., solid phase peptide synthesis).

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. In another aspect, a string of amino acids can be conservatively replaced with a structurally similar string that differs in order and/or composition of side chain family members.

Non-conservative amino acid substitutions include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

Other amino acid substitutions can also be used. For example, for the amino acid alanine, a substitution can be taken from any one of D-alanine, glycine, beta-alanine, L-cysteine and D-cysteine. For lysine, a replacement can be any one of D-lysine, arginine, D-arginine, homo-arginine, methionine, D-methionine, ornithine, or D-ornithine. Generally, substitutions in functionally important regions that can be expected to induce changes in the properties of isolated polypeptides are those in which (i) a polar residue, e.g., serine or threonine, is substituted for (or by) a hydrophobic residue, e.g., leucine, isoleucine, phenylalanine, or alanine; (ii) a cysteine residue is substituted for (or by) any other residue; (iii) a residue having an electropositive side chain, e.g., lysine, arginine or histidine, is substituted for (or by) a residue having an electronegative side chain, e.g., glutamic acid or aspartic acid; or (iv) a residue having a bulky side chain, e.g., phenylalanine, is substituted for (or by) one not having such a side chain, e.g., glycine. The likelihood that one of the foregoing non-conservative substitutions can alter functional properties of the protein is also correlated to the position of the substitution with respect to functionally important regions of the protein: some non-conservative substitutions can accordingly have little or no effect on biological properties.

In the content of the present disclosure, the terms “mutation” and “amino acid substitution” as defined above (sometimes referred simply as a “substitution”) are considered interchangeable.

### II. SETD7 Modulators

The present disclosure provides epigenetic modulators of histone-lysine N-methyltransferase SETD7 (“SETD7 modulators”). As used herein, the terms “SETD7 modulator,” “SETD7 modulator of the present disclosure” and grammatical variants thereof refer to molecules that can modulate, e.g., monomethylase activity of SEDT7 in the nucleus of a target cell. Accordingly, the term encompasses, e.g., SETD7 modulator polypeptides (e.g., polypeptides competing with SETD7 for binding to importins and therefore reducing SETD7 nuclear transport and accumulation), polynucleotides encoding such SETD7 modulator polypeptides, vectors (e.g., vectors comprising polynucleotides encoding SETD7 modulator polypeptides), micelles (e.g., micelles comprising SETD7 modulator polypeptides, polynucleotides or vectors), and/or cells (e.g., cell comprising SETD7 modulator polypeptides, polynucleotides or vectors). The term also encompasses molecules capable of specifically binding to endogenous SETD7 protein or endogenous mRNA encoding SETD7, e.g., antibodies, aptamers, or antisense oligonucleotides. In some particular aspects, the term refers to, e.g., SETD7-derived polypeptides (synthetic or recombinant peptides corresponding to the C-terminal region of SETD7), polynucleotides encoding such polypeptides, catalytically inactive mutant forms of SETD7 (i.e., mutants without monomethylase activity or with significantly reduced monomethylase activity), and polynucleotides encoding such mutant forms of SETD7.

Histone-lysine N-methyltransferase SETD7 (“SETD7,” also known as SET7/9) is a histone methyltransferase that specifically monomethylates ‘Lys-4’ of histone H3. H3 ‘Lys-4’ methylation represents a specific tag for epigenetic transcriptional activation. SETD7 plays a central role in the transcriptional activation of genes such as collagenase or insulin. SETD7 is recruited by IPF1/PDX-1 (insulin promoter factor 1, also known as pancreatic and duodenal homeobox 1) to the insulin promoter, leading to activate transcription. SETD7 has also methyltransferase activity toward non-histone proteins such as p53/TP53 (tumor protein p53, and particularly its human form TP53), TAF10 (Transcription initiation factor TFIID subunit 10), and possibly TAF7 (Transcription initiation factor TFIID subunit 7) by recognizing and binding the [KR]-[STA]-K amino acid motif in substrate proteins. In particular, SETD7 monomethylates ‘Lys-189’ of TAF10, leading to increase the affinity of TAF10 for RNA polymerase II. Also, SETD7 monomethylates ‘Lys-372’ of p53/TP53, stabilizing p53/TP53 and increasing p53/TP53-mediated transcriptional activation. See, Martens et al. (2003) Mol. Cell. Biol. 23:1808-1816; Kouskouti et al. (2004) Mol. Cell 14:175-182; Francis et al. (2005) J. Biol. Chem. 280:36244-53; Huang et al. (2006) Nature 444:629-32; Xiao et al. (2003) Nature 421: 652-56; and Chuikov et al. (2004) Nature 432:353-60, all of which are herein incorporated.

SETD7 (Uniprot Q8WTS6) is the only lysine methyltransferase (KMT)7 family member due to its unique enzymatic activity and protein domain architecture. Duan et al. (2018) EBiomedicine 37:134-143. Recent investigations indicate that SETD7 is potentially a biomarker for hepatocellular cancer and breast cancer. SETD7 expression increases from healthy individuals to those with colorectal polyps and finally colorectal cancer (CRC) patients. As a methyltransferase, numerous non-histone substrates including transcription factors have been described for SETD7, including E2F1 (transcription factor E2F1), MINT (Msx2-interacting protein), IRF1 (interferon regulatory factor 1), TAF7 and CENPC1 (centromere protein C1) and others. See Kim et al. (2016) Nat. Commun, 7:19347. SETD7 is involved in cancer, e.g., by regulating the Wnt/β-catenin and Hippo/YAP pathways. See, e.g., Montenegro et al. (2016) Oncogene 35:6143-52; Oudhoff et al. (2016) Dev. Cell 37:47-57; Shen et al. (2015) FASEB J. 29:4313-4323; and Coghlin & Murray (2015) Proteomics Clin. Appl. 9: 64-71, which are herein incorporated by reference in their entireties. SETD7 overexpression inhibits the nuclear translocation of NF-κB p65 in hepatocytes during hepatitis C virus replication. Notably, SETD7 has been reported to contribute to TNF-α-induced methylation of NF-κB p65 subunit p65, which is required for the expression of a subset of NF-κB target genes in response to TNF-α stimulation. Wu et al. (2019) International Immunopharmacology 72: 459-66.

SETD7 is also responsible for hyperglycemic memory when cells are exposed to transient high glucose concentrations. Exposure to high glucose induces translocation of SETD7 into the nucleus and subsequently leads to histone methylation/gene action events. SETD7 does not contain a nuclear localization signal. However, SETD7 contains several phosphorylatable amino acids in the C-terminal region. Thus, nuclear translocation in response, e.g., to hyperglycemia, is tightly regulated by posttranslational modification (e.g., phosphorylation).

In some aspects, the present disclosure comprises a SETD7 modulator that combines a phosphomimicking mutant and a catalytically inactive SETD7 mutant. In some aspects, a SETD7 modulator of the present disclosure comprises an anti-SETD7 antibody that binds specifically to endogenous SETD7.

### II.a SETD7 Phosphomimicking Modulator

The present disclosure comprises a SETD7 modulator that can competitively inhibit endogenous SETD7, thereby preventing the endogenous SETD7 from being transported into the nucleus. In some aspects, the disclosure comprises an RNA aptamer that specifically binds to the region of SETD7 gene, which encodes a C-terminus portion of the endogenous SETD7 protein In some aspects of the present disclosure the SETD7 modulator comprises, consists, or consists essentially of a polynucleotide encoding a polypeptide (e.g., a recombinant or a synthetic polypeptide) or a polypeptide comprising at least one phosphorylatable amino acid selected from Ser225, Tyr305, Thr332, Ser340, and Ser345 or the polypeptide encoded by the polynucleotide. In some aspects, the polypeptide (e.g., a recombinant or a synthetic polypeptide) comprises the SET domain of SETD7 or a portion thereof, or a sequence located between the C-terminus of the SETD7 SET domain and the C-terminus of SETD7, wherein the polypeptide comprises at least one of Ser225, Tyr305, Thr332, Ser340, and Ser345. In some aspects, these nonphosphorylated peptides can compete with endogenous SETD7 for binding to protein kinases.

In some aspects of the present disclosure the SETD7 modulator comprises, consists, or consists essentially of a polynucleotide encoding a polypeptide (e.g., a recombinant polypeptide or a synthetic polypeptide) or a polypeptide comprising at least one phosphorylatable amino acid selected from Ser225, Tyr305, Thr332, Ser340, and Ser345 or the polypeptide encoded by the polynucleotide, wherein at least one phosphorylatable amino acid (Ser225, Tyr305, Thr332, Ser340, or Ser345) or a combination thereof has been replaced (i.e., substituted) with a phosphomimetic amino acid (e.g., Asp or Glu), a Ser, Tyr, or Thr analog (e.g., a non-hydrolyzable analog), or any combination thereof. These peptides comprising phosphomimicking amino acids would mimic SETD7 in a phosphorylated state and compete with endogenous phosphorylated SETD7, e.g., for binding to a shuttling protein, thus reducing the translocation of phosphorylated SETD7 to the nucleus.

As used herein, the term “recombinant polypeptide” refers to a peptide, polypeptide, or protein that results from the expression of a recombinant nucleic acid (e.g., recombinant DNA) within living cells.

As used herein, the term “synthetic polypeptide” refers to a peptide, polypeptide, or protein that is formed, in vitro, by joining amino acids or amino acid analogs in a particular order, using well known techniques of synthetic organic peptide synthesis to form the peptide bonds, e.g., via solid phase peptide synthesis.

Thus, in some aspects, the present disclosure provides an isolated polynucleotide (a SETD7 modulator polynucleotide) encoding a polypeptide (a SETD7 modulator polypeptide) which comprises the sequence X₁VAYGYDHX₂PPGKX₃ (SEQ ID NO: 1), wherein each of X₁, X₂, and X₃ is

### (i) Serine (S) or Threonine (T);

(ii) a phosphomimetic amino acid or analog thereof; or,
(iii) a combination thereof,
wherein the polypeptide sequence is not TVAYGYDHSPPGKS (SEQ ID NO: 2; wild type), wherein one, two, three, four or five amino acids other than X₁, X₂, and X₃ are optionally substituted with respect to their corresponding amino acids in SEQ ID NO: 2, and wherein the polypeptide is capable of modulating nuclear translocation of endogenous SETD7.

In some aspects, the present disclosure provides a polypeptide (a SETD7 modulator polypeptide, e.g., a recombinant polypeptide, synthetic polypeptide, or polypeptide encoded by SETD7 modulator polynucleotide) which comprises the sequence X₁VAYGYDHX₂PPGKX₃ (SEQ ID NO: 1), wherein each of X₁, X₂, and X₃ is

### (i) Serine (S) or Threonine (T);

(ii) a phosphomimetic amino acid or analog thereof; or,
(iii) a combination thereof,
wherein the polypeptide sequence is not TVAYGYDHSPPGKS (SEQ ID NO: 2; wild type), wherein one, two, three, four or five amino acids other than X₁, X₂, and X₃ are optionally substituted with respect to their corresponding amino acids in SEQ ID NO: 2, and wherein the polypeptide is capable of modulating nuclear translocation of endogenous SETD7.

As used herein the term “phosphomimetic amino acid” refers to an amino acid that mimics a phosphorylated amino acid. Within cells, proteins are commonly modified at serine, threonine, and tyrosine amino acids by adding a phosphate group. However, some non-phosphorylated amino acids appear chemically similar to phosphorylated amino acids. For example, aspartic acid is chemically similar to phospho-serine. Thus, when an aspartic acid replaces a serine, it is a phosphomimetic of phospho-serine and can make the protein function like it was in its phosphorylated form.

As used herein, SEQ ID NO:2 refers to a wild type SETD7 isolated subsequence comprising the amino acids from Thr332 to Ser345, wherein the N-terminal amino acid comprises a free N-terminal amino group, and the C-terminal amino acid comprises a free C-terminal carboxyl group.

In some aspects, the phosphomimetic amino acid is aspartic acid (D) or glutamic acid (E). In some aspects, the aspartic acid is L aspartic acid. In other aspects, the aspartic acid is D aspartic acid. In some aspects, the glutamic acid is L glutamic acid. In other aspects, the glutamic acid is D glutamic acid. In some aspects, the phosphomimetic amino acid is phosphoserine or phosphothreonine. In some aspects, the phosphomimetic amino acid is L phosphoserine, D phosphoserine, L phosphothreonine, or D phosphothreonine.

In some aspects, the phosphomimetic amino acid analog is a non-cleavable analog, i.e., an amino acid analog having a group mimicking a phosphate group, wherein the mimicking group cannot be hydrolyzed by phosphatases and/or other enzymes. In some aspects, the non-cleavable analog is, e.g., a phosphoserine non-hydrolyzable analog. In some aspects, the non-hydrolyzable analog of phosphoserine is, e.g., 2-amino-4-phosphobutyric acid.

In some aspects, the optionally substituted amino acids are conservative amino acid substitutions. A conservative amino acid substitution is one conducted according to similar biochemical or biophysical properties. Thus, the types of allowable substitutions would depend on the particular property, such a charge, hydrophobicity, or size. In some aspects, an aliphatic amino acid (e.g., V, G, A) is replaced with another aliphatic amino acid (e.g, V, G, A, I, L). In some aspects, an aromatic amino acid (e.g., Y) is replaced with F, or W. In some aspects, a basic amino acid (e.g., H) is replaced with K or R. In some aspects, an acidic amino acid (e.g., D) is replaced with E, N, or Q.

In some aspects, at least one tyrosine (Y) is substituted. In some aspects, at least one tyrosine is substituted, e.g., with phosphotyrosine, aspartic acid, glutamic acid, or an analog thereof. In some aspects, the substituting amino acid is an L amino acid. In some aspects, the substituting amino acid is a D amino acid. In some aspects, the phosphotyrosine analog is, e.g., a non-hydrolyzable analog. In some aspects, the non-hydrolyzable analog of phosphotyrosine is, e.g., 4-phosphomethyl-L-phenylalanine (Pmp).

In some aspects, the phosphomimetic amino acid analog is, e.g., a thiophosphate analog. In some aspects, the thiophosphate analog is, e.g., thiophosphoserine. In some aspects, the phosphomimetic amino acid analog is, e.g., a sulfate analog. In some aspects, the sulfate analog is, e.g., sulfoserine.

In some aspects, the sequence of the polypeptide (a SETD7 modulator polypeptide, e.g., a recombinant polypeptide, synthetic polypeptide, or polypeptide encoded by SETD7 modulator polynucleotide) comprises a sequence selected from the group consisting of SEQ ID NOS: 3, 4, 5, 6, 7, 8, 9, 10, 11, 12, 13, 14, 15, 16, 17 and 18.

In some aspects, a polypeptide set forth in SEQ ID NO:3 (x-TVAYGYDHSPPGKS-y) comprises N- and/or C-terminal modification, wherein “x” is an N-terminal modification and “y” is a C-terminal modification (e.g., a capping modification). “x” and “y” are peptide endings different from those found in a naturally occurring isolated polypeptide.

Capping modifications are generally introduced at the termini of chemically synthesized peptides to increase their resistance to proteolytic degradation. In some aspects, the N-terminal modification is acetylation. See, e.g., Thomas (2011) PLOS Biol. 9; Wallace (1992) Br. J. Nutr. 68:365-72. This modification removes the positive charge of the N-terminal of peptides, thus mimicking natural proteins and increasing peptide stability by preventing N-terminal degradation. In some aspects, the C-terminal modification is amidation. This modification neutralizes the negative charge created by the C-terminal COOH. This modification is added to prevent enzyme degradation, to mimic native proteins, and in some cases to remove hydrogen bonding at the C-terminal of the peptides. See, e.g., Kim et al. (2001) Biotechnol. Bioprocess Eng. 6:244-51.

In some aspects, the polynucleotide encodes a polypeptide (a SETD7 modulator polypeptide, e.g., a recombinant polypeptide, synthetic polypeptide, or polypeptide encoded by SETD7 modulator polynucleotide) that comprises the sequence

(SEQ ID NO: 19)
X₄VAX₅GX₆DHX₇PPGKX₈

wherein X₄, X₇ and X₈ are selected from the group consisting of serine, threonine, aspartic acid, glutamic acid, 2-amino-4-phosphobutyric acid, and thiophosphoserine; and wherein X₅ and X₆ are selected from the group consisting of tyrosine, and 4-phosphomethyl-L-phenylalanine. In some aspects, the polypeptide further comprises (i) an L, EL, EEL, DEEL (SEQ ID NO:20), or ADEEL (SEQ ID:21) amino acid sequence appended to its N-terminus; (ii) a G, GP, GPE, GPEA (SEQ ID NO:22), or GPEAP (SEQ ID NO:23) amino acid sequence appended to its C-terminus; or, (iii) a combination thereof.

Thus, in some aspects, the polypeptide encoded by the polynucleotide of the disclosure (a SETD7 modulator polypeptide, e.g., a recombinant polypeptide, synthetic polypeptide, or polypeptide encoded by SETD7 modulator polynucleotide) comprises a sequence set forth in SEQ ID NO:19 which is derived from the SETD7 amino acid subsequence from Thr332 to Ser345, and wherein any of the phosphorylatable amino acids (Ser225, Tyr305, Thr332, Ser340, and Ser345) or any combination thereof is replaced with a phosphomimic amino acid, with a non-cleavable analog, or any combination thereof. In some aspects, the polypeptide of SEQ ID NO:19 is flanked on its N-terminus by 1, 2, 3, 4, or 5 additional amino acids. In some aspects, the 1, 2, 3, 4, or 5 additional N-terminal amino acids are corresponding native amino acids from the wild type sequence of SETD7. In some aspects, the polypeptide of SEQ ID NO: 19 is flanked on its N-terminus by 1, 2, 3, 4, or 5 additional amino acids on its C-terminus. In some aspects, the 1, 2, 3, 4, or 5 additional C-terminal amino acids are corresponding native amino acids from the wild type sequence of SETD7.

In some aspects, the polypeptide of SEQ ID NO:19 is flanked by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 additional amino acids on its N-terminus and/or by 1, 2, 3, 4, 5, 6, 7, 8, 9, 10, 11, 12, 13, 14, 15, 16, 17, 18, 19, 20, or 21 additional amino acids on its C-terminus.

In some aspects, the polypeptide encoded by the polynucleotide of the disclosure (a SETD7 modulator polypeptide, e.g., a recombinant polypeptide, synthetic polypeptide, or polypeptide encoded by SETD7 modulator polynucleotide) comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence as set forth in SEQ ID NOS: 24, 25, 26, 27, 28, 29, 30 or 31.

In some aspects, the polypeptide encoded by the polynucleotide of the disclosure (a SETD7 modulator polypeptide, e.g., a recombinant polypeptide, synthetic polypeptide, or polypeptide encoded by SETD7 modulator polynucleotide) consists of or consists essentially of the sequence as set forth in SEQ ID NOS: 24, 25, 26, 27, 28, 29, 30 or 31.

In some aspects, the polypeptide encoded by the polynucleotide of the disclosure (a SETD7 modulator polypeptide, e.g., a recombinant polypeptide, synthetic polypeptide, or polypeptide encoded by SETD7 modulator polynucleotide) consists of or consists essentially of the sequence as set forth in SEQ ID NOS: 24, 25, 26, 27, 28, 29, 30 or 31, except for 1, 2, 3, 4 or 5 amino acid substitutions, e.g., conservative amino acid substitutions.

The present disclosure also provides an isolated polynucleotide (a SETD7 modulator polynucleotide) encoding a polypeptide (a SETD7 modulator polypeptide) which consists of or consists essentially of the sequence as set forth in SEQ ID NO: 2 and (i) an L, EL, EEL, DEEL (SEQ ID NO:20), or ADEEL (SEQ ID:21) amino acid sequence appended to its N-terminus; (ii) a G, GP, GPE, GPEA (SEQ ID NO:22), or GPEAP (SEQ ID NO:23) amino acid sequence appended to its C-terminus; or, (iii) a combination thereof.

In some aspects, the polypeptide (a SETD7 modulator polypeptide, e.g., a recombinant polypeptide, synthetic polypeptide, or polypeptide encoded by SETD7 modulator polynucleotide) consists essentially of or consists of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence as set forth in ADEELTVAYGYDHSPPGKSGPEAP (SEQ ID NO:32).

In some aspects, the polypeptide encoded by the polynucleotide of the disclosure (a SETD7 modulator polypeptide, e.g., a recombinant polypeptide, synthetic polypeptide, or polypeptide encoded by SETD7 modulator polynucleotide) consists or consists essentially of ADEELTVAYGYDHSPPGKSGPEAP (SEQ ID NO:32). ADEELTVAYGYDHSPPGKSGPEAP (SEQ ID NO:32) except for except for 1, 2, 3, 4 or 5 amino acid substitutions, e.g., conservative amino acid substitutions.

In some aspects, the polypeptide encoded by the polynucleotide of the disclosure (a SETD7 modulator polypeptide, e.g., a recombinant polypeptide, synthetic polypeptide, or polypeptide encoded by SETD7 modulator polynucleotide) has at least 14 amino acids, at least 15 amino acids, at least 16 amino acids, at least 17 amino acids, at least 18 amino acids, at least 19 amino acids, at least 20 amino acids, at least 21 amino acids, at least 22 amino acids, at least 23 amino acids, or at least 24 amino acids in length. In some aspects, the polypeptide has at least 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, or 155 amino acids in length.

In some aspects, the polypeptide encoded by the polynucleotide of the disclosure (a SETD7 modulator polypeptide, e.g., a recombinant polypeptide, synthetic polypeptide, or polypeptide encoded by SETD7 modulator polynucleotide) comprises at least about 20 amino acids, at least about 30 amino acids, at least about 40 amino acids, at least about 50 amino acids, at least about 60 amino acids, at least about 70 amino acids, at least about 80 amino acids, at least about 90 amino acids, at least about 100 amino acids, at least about 120 amino acids, at least about 140 amino acids, at least about 160 amino acids, at least about 180 amino acids, at least about 200 amino acids, at least about 220 amino acids, at least about 240 amino acids, at least about 260 amino acids, at least about 280 amino acids, at least about 300 amino acids, at least about 320 amino acids, at least about 340 amino acids, at least about 360 amino acids in length, or about 366 amino acids in length.

In some aspects, the polypeptide encoded by the polynucleotide of the disclosure (a SETD7 modulator polypeptide, e.g., a recombinant polypeptide, synthetic polypeptide, or polypeptide encoded by SETD7 modulator polynucleotide) comprises an N-terminal capping modification, a C-terminal capping modification, or a combination thereof. In some aspects, the N-terminal capping modification is an N-terminal acetylation, formylation, acylation, pyroglutamylation, or carbamate, sulfonamide, or alkylamine modification. In some aspects, the C-terminal capping modification is a C-terminal amidation, N-alkyl amidation, aldehyde modification, or esterification.

In some aspects, the isolated polynucleotide (a SETD7 modulator polynucleotide) comprises a nucleic acid sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence as set forth in 5′-GCNGAYGARGARYUNGARGUNGCNUAYGGNUAYGAYCAYGAYCCNCCNGGNAAR GAYGGNCCNGARGCNCCNURR-3′ (SEQ ID NO:33), wherein N is any nucleotide (e.g., A, G, T, U, or C), Y is a pyrimidine (e.g., C, T, or U), R is a purine (e.g., A or G). In some aspects, the sequence is a DNA sequence. In some aspects, the sequence is an RNA sequence. In some aspects, the sequence is an RNA/DNA sequence.

In some aspects, the isolated polynucleotide (a SETD7 modulator polynucleotide) comprises a nucleic acid sequence as set forth in 5′-GCNGAYGARG ARYUNGARGUNGCNUAYGGNUAYGAYCAYGAYCCNCCNGGNAARGAYGGNCCN GARGCNCCNURR-3′ (SEQ ID NO:33), wherein N is any nucleotide (e.g., A, G, T, U, or C), Y is a pyrimidine (e.g., C, T, or U), R is a purine (A or G). In some aspects, the sequence is a DNA sequence. In some aspects, the sequence is an RNA sequence. In some aspects, the sequence is an RNA/DNA sequence.

In some aspects, the isolated polynucleotide (a SETD7 modulator polynucleotide) consists of or consists essentially of a nucleic acid sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence as set forth in 5′-GCNGAYGARGARYUNACNGUNGCNUAYGGNUAYGAYCAYWSNCCNCCNGGNAAR WSNGGNCCNGARGCNCCNURR-3′ (SEQ ID NO: 34), wherein N is any nucleotide (e.g., A, G, T, U, or C), Y is a pyrimidine (e.g., C, T, or U), R is a purine (A or G), W is a weak bonding nucleotide (e.g., A, T or U) and S is a strong bonding nucleotide (e.g., G or C). In some aspects, the sequence is an RNA sequence. In some aspects, the sequence is an RNA/DNA sequence. In some aspects, the polynucleotide encoding a SETD7 modulator is mRNA. In some aspects, the polynucleotide encoding a SETD7 modulator of the present disclosure is a DNA. In some aspects, the polynucleotide encoding a SETD7 modulator is inserted in a vector.

### II.b SETD7 Catalytically Inactive Molecules

In some aspects, the present disclosure provides a polynucleotide encoding a SETD7 mutant protein or a fragment thereof wherein the SETD7 mutant protein is enzymatically inactive or has significantly reduced methyltransferase activity (e.g., histone monomethyl transferase activity). In some aspects, the present disclosure provides a SETD7 mutant protein or a fragment thereof wherein the SETD7 mutant protein is enzymatically inactive or has significantly reduced methyltransferase activity (e.g., histone monomethyl transferase activity). The present SETD7 mutant protein or a fragment thereof wherein the SETD7 mutant protein is enzymatically inactive or has significantly reduced methyltransferase activity (e.g., histone monomethyl transferase activity).

Such mutant SETD7 proteins would be phosphorylated as wild type SETD7, but after their translocation to the nucleus they would be catalytically inactive. Furthermore, phosphorylation of the mutant SETD7 proteins would lead to overall lower phosphorylation levels of wild type, and the phosphorylated mutant SETD7 protein would compete with wild type phosphorylated SETD7, e.g., for binding to importins, thus reducing the overall amount of phosphorylated wild type SETD7 reaching the nucleus.

As used herein, the term “SETD7 mutant protein” refers to a SETD7 protein which does not perform its usual or normal physiological role, e.g., lacking or having severely reduced enzymatic activity, or having an impaired ability to be translocated to the nucleus with respect to endogenous normal SETD7. The term “normal” means a protein which performs its usual or normal physiological role. Therefore, as used herein, the term “normal” is essentially synonymous with the usual meaning of the phrase “wild type.”

In some aspects, the SETD7 mutant protein comprises a His297 mutation. The mutations of the Histidine at position 297 results in the loss of enzymatic activity. See Nishioka et al. (2002) Genes Dev. 16:479-89, which is herein incorporated by reference in its entirety. The terminal carboxyl group of the S-adenosyl-L-methionine cofactor interacts with the amide nitrogen of Ser225 while its amine nitrogen is hydrogen bonded to the carbonyl oxygen of His297. Other amino acids susceptible of mutation in order to abolish the methyltransferase activity of SETD7 are disclosed in Wilson et al. (2002) Cell 111:105-115, which is herein incorporated by reference in its entirety. The residues immediately adjacent to the C-terminus of the SET domain (located between amino acid positions 214 and 336) are important for the catalytic competence of the SETD7 protein but not for substrate or cofactor binding.

Mutagenic studies on the C-terminus of SETD7 have shown that residues located C-terminal to Lys344 are essential for catalytic activity and are likely to be in contact with the active site of the enzyme. For example, deletion of the C-terminal region (position 344 to C-terminus at position 366), and substitutions at positions 245 (e.g., Y245A), 254 (e.g., E254A), 270 (e.g., D270A), 296 (e.g., N296A), 297 (e.g., H297A), 302 (e.g., N302A), 329 (e.g., E329A), 330 (e.g., E330A), or 335 (e.g., Y335A) result in partial or complete loss of histone monomethyl transferase activity.

Mutation at positions 352 (e.g., W352A) or 353 (e.g., Y353A) also led to a complete loss of monomethyl transferase activity, and mutations at positions 351 (e.g., E351A), 354 (e.g., Q354A), 356 (e.g., E356A), 358 (e.g., K358A), 361 (e.g., Q361A), 365 (e.g., Q365A) or 366 (e.g., K366A) result in a partial loss of monomethyl transferase activity.

Whereas the H297A mutation abolishes methyltransferase activity, the K317A mutation induces a reduction in methyltransferase activity toward TAF10 but an increased methyltransferase activity for H3 and p53/TP53, and the Y245A mutation significantly reduces the monomethyltransferase activity but increases the dimethyltransferase activity.

Other possible sites of intervention to modulate the catalytic activity of SETD7 are its substrate binding sites (Tyr245, Lys317, and Tyr335) and its S-adenosyl-L-methionine binding site (Glu356). K294A mutation reduces catalytic activity (e.g., monomethyl transferase activity).

Accordingly, in some aspects, the SETD7 mutant protein of the present disclosure comprises (i) a C-terminal deletion (at least 1, 2, 3, 4, 5, 6, 7, 8, 9, 10, 11, 12, 13, 14, 15, 16, 17, 18, 19, 20, 21, or 22 C-terminal amino acids deleted), or (ii) a substitution at position 225, 245, 254, 270, 294, 296, 297, 302, 317, 329, 330, 335, 351, 352, 353, 354, 356, 358, 361, 365, 366, or any combination thereof, or (iii) any combination thereof, wherein the mutations (i.e., deletions and/or substitutions) result in a decrease or loss of monomethyl transferase activity (e.g., histone monomethyl transferase activity).

In a specific aspect, SETD7 mutant protein of the present disclosure comprise a H297A mutation. In some aspects, the SETD7 mutant protein of the present disclosure comprises (i) a H297A mutation and (ii) at least one phosphomimetic substitution at position 332, 340, 345, or any combination thereof. In some aspects, the SETD7 mutant protein of the present disclosure comprises (i) a H297A mutation and (ii) one phosphomimetic substitution at position 332, 340, or 345. In some aspects, the SETD7 mutant protein of the present disclosure comprises (i) a H297A mutation and (ii) two phosphomimetic substitutions at positions 332 and 340, positions 332 and 345, or positions 340 and 345. In some aspects, the SETD7 mutant protein of the present disclosure comprises (i) a H297A mutation and (ii) two phosphomimetic substitutions at positions 332, 340, and 345.

In some aspects, the SETD7 mutant protein of the present disclosure has a monomethyl transferase activity reduced to about 90%, about 85%, about 80%, about 75%, about 70%, about 65%, about 60%, about 55%, about 50%, about 45%, about 40%, about 35%, about 30%, about 25%, about 20%, about 15%, about 10%, about 5%, or about 0% of the monomethyl transferase activity of wild type SETD7 as measured, e.g., as disclosed in Wilson et al. (2002) Cell 111:105-115.

In some aspects, the SETD7 mutant protein is at least about 120 amino acids, at least about 125 amino acids, at least about 130 amino acids, at least about 135 amino acids, at least about 140 amino acids, at least about 145 amino acids, at least about 150 amino acids, at least about 155 amino acids, at least about 160 amino acids, at least about 165 amino acids, at least about 170 amino acids, at least about 175 amino acids, at least about 180 amino acids, at least about 185 amino acids, at least about 190 amino acids, at least about 195 amino acids, at least about 200 amino acids, at least about 205 amino acids, at least about 210 amino acids, at least about 215 amino acids, at least about 220 amino acids, at least about 225 amino acids, at least about 230 amino acids, at least about 235 amino acids, at least about 240 amino acids, at least about 245 amino acids, at least about 250 amino acids, at least about 255 amino acids, at least about 260 amino acids, at least about 265 amino acids, at least about 270 amino acids, at least about 275 amino acids, at least about 280 amino acids, at least about 285 amino acids, at least about 290 amino acids, at least about 295 amino acids, at least about 300 amino acids, at least about 305 amino acids, at least about 310 amino acids, at least about 315 amino acids, at least about 320 amino acids, at least about 325 amino acids, at least about 330 amino acids, at least about 335 amino acids, at least about 340 amino acids, at least about 345 amino acids, at least about 350 amino acids, at least about 355 amino acids, at least about 360 amino acids, or about 366 amino acids in length.

In some aspects, the SETD7 mutant protein of the present disclosure comprises at least about 70%, at least about 75%, at least about 80%, at least about 85%, at least about 90%, at least about 95%, at least about 96%, at least about 97%, at least about 98%, at least about 99%, or about 100% sequence identity to the sequence as set forth in SEQ ID NO: 35. In some aspects, the SETD7 mutant protein of the present disclosure consists of or consists essentially of the sequence as set forth in SEQ ID NO: 35.

In some aspects, the SETD7 mutant protein comprises at least about 120 contiguous amino acids, at least about 125 contiguous amino acids, at least about 130 contiguous amino acids, at least about 135 contiguous amino acids, at least about 140 contiguous amino acids, at least about 145 contiguous amino acids, at least about 150 contiguous amino acids, at least about 155 contiguous amino acids, at least about 160 contiguous amino acids, at least about 165 contiguous amino acids, at least about 170 contiguous amino acids, at least about 175 contiguous amino acids, at least about 180 contiguous amino acids, at least about 185 contiguous amino acids, at least about 190 contiguous amino acids, at least about 195 contiguous amino acids, at least about 200 contiguous amino acids, at least about 205 contiguous amino acids, at least about 210 contiguous amino acids, at least about 215 contiguous amino acids, at least about 220 contiguous amino acids, at least about 225 contiguous amino acids, at least about 230 contiguous amino acids, at least about 235 contiguous amino acids, at least about 240 contiguous amino acids, at least about 245 contiguous amino acids, at least about 250 contiguous amino acids, at least about 255 contiguous amino acids, at least about 260 contiguous amino acids, at least about 265 contiguous amino acids, at least about 270 contiguous amino acids, at least about 275 contiguous amino acids, at least about 280 contiguous amino acids, at least about 285 contiguous amino acids, at least about 290 contiguous amino acids, at least about 295 contiguous amino acids, at least about 300 contiguous amino acids, at least about 305 contiguous amino acids, at least about 310 contiguous amino acids, at least about 315 contiguous amino acids, at least about 320 contiguous amino acids, at least about 325 contiguous amino acids, at least about 330 contiguous amino acids, at least about 335 contiguous amino acids, at least about 340 contiguous amino acids, at least about 345 contiguous amino acids, at least about 350 contiguous amino acids, at least about 355 contiguous amino acids, at least about 360 contiguous amino acids, or about 366 contiguous amino from the sequence as set forth in SEQ ID NO: 35.

In some aspects, the isolated polynucleotide encoding a SETD7 mutant protein of the present disclosure comprises or consists of the sequence set forth in SEQ ID NO: 36, wherein N is any nucleotide (A,G,T,C), Y is a pyrimidine (C,T), and R is a purine (A,G).

In some aspects, the isolated polynucleotide encoding a SETD7 mutant protein of the present disclosure comprises or consists of a fragment or variant of the sequence set forth in SEQ ID NO: 39, wherein the fragment or variant encodes a SETD7 mutant protein with a decrease or loss of monomethyl transferase activity (e.g., histone monomethyl transferase activity) with respect to wild type SETD7.

In some aspects, the isolated polynucleotide encoding a SETD7 mutant protein of the present disclosure is identical to SEQ ID NO: 39, except for 1, 2, 3, 4, 5, 6, 7, 8, 9, or 10 mutations (base substitutions), wherein the polynucleotide encodes a SETD7 mutant protein with a decrease or loss of monomethyl transferase activity (e.g., histone monomethyl transferase activity) with respect to wild type SETD7.

In some aspects, the isolated polynucleotide encoding a SETD7 mutant protein of the present disclosure comprises a nucleotide sequence at least about 50%, at least about 55%, at least about 60%, at least about 65%, at least about 70%, at least about 75%, at least about 80%, at least about 85%, at least about 90%, at least about 95%, or at least about 99% identical to the sequence set forth in SEQ ID NO: 39, wherein the nucleotide sequence encodes a SETD7 mutant protein with a decrease or loss of monomethyl transferase activity (e.g., histone monomethyl transferase activity) with respect to wild type SETD7.

In some aspects, the SETD7 mutant protein comprises the amino acid sequence set forth in SEQ ID NO: 37, except for 1, 2, 3, 4, 5, 6, 7, 8, 9, or 10 amino acid substitutions, wherein the SETD7 mutant protein has a decrease or loss of monomethyl transferase activity (e.g., histone monomethyl transferase activity) with respect to wild type SETD7.

In some aspects, the SETD7 mutant protein comprises an amino acid at least about 60%, at least about 65%, at least about 70%, at least about 75%, at least about 80%, at least about 85%, at least about 90%, at least about 95%, or at least about 99% identical to the amino acid sequence set forth in SEQ ID NO: 37, except for 1, 2, 3, 4, 5, 6, 7, 8, 9, or 10 amino acid substitutions, wherein the SETD7 mutant protein has a decrease or loss of monomethyl transferase activity (e.g., histone monomethyl transferase activity) with respect to wild type SETD7.

### II.c SETD7 Gene Inactivation

In some aspects, SETD7 gene can be inactivated by various methods, e.g., siRNA, antisense oligonucleotide, a gene editing tool, or any combination thereof. In some aspects, SETD7 gene can be inactivated by using one or more gene editing tools. One or more gene editing tools can be used to modify the cells of the present disclosure. Non-limiting examples of the gene editing tools are disclosed below:

### II.c.i CRISPR/Cas System

In some aspects, the gene editing tool that can be used in the present disclosure comprises a CRISPR/Cas system. Such systems can employ, for example, a Cas9 nuclease, which in some instances, is codon-optimized for the desired cell type in which it is to be expressed (e.g., T cells, e.g., CAR-expressing T cells). CRISPR/Cas systems use Cas nucleases, e.g., Cas9 nucleases, that are targeted to a genomic site by complexing with a synthetic guide RNA (gRNA) that hybridizes to a target DNA sequence immediately preceding an NGG motif recognized by the Cas nuclease, e.g., Cas9. This results in a double-strand break three nucleotides upstream of the NGG motif. A unique capability of the CRISPR/Cas9 system is the ability to simultaneously target multiple distinct genomic loci by co-expressing a single Cas9 protein with two or more gRNAs (e.g., at least one, two, three, four, five, six, seven, eight, nine or ten gRNAs). Such systems can also employ a guide RNA (gRNA) that comprises two separate molecules. In certain aspects, the two-molecule gRNA comprises a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA” or “scaffold”) molecule.

A crRNA comprises both the DNA-targeting segment (single stranded) of the gRNA and a stretch of nucleotides that forms one half of a double stranded RNA (dsRNA) duplex of the protein-binding segment of the gRNA. A corresponding tracrRNA (activator-RNA) comprises a stretch of nucleotides that forms the other half of the dsRNA duplex of the protein-binding segment of the gRNA. Thus, a stretch of nucleotides of a crRNA is complementary to and hybridizes with a stretch of nucleotides of a tracrRNA to form the dsRNA duplex of the protein-binding domain of the gRNA. As such, each crRNA can be said to have a corresponding tracrRNA. The crRNA additionally provides the single stranded DNA-targeting segment. Accordingly, a gRNA comprises a sequence that hybridizes to a target sequence (e.g., SETD7 mRNA), and a tracrRNA. Thus, a crRNA and a tracrRNA (as a corresponding pair) hybridize to form a gRNA. If used for modification within a cell, the exact sequence and/or length of a given crRNA or tracrRNA molecule can be designed to be specific to the species in which the RNA molecules will be used (e.g., humans).

Naturally-occurring genes encoding the three elements (Cas9, tracrRNA and crRNA) are typically organized in operon(s). Naturally-occurring CRISPR RNAs differ depending on the Cas9 system and organism but often contain a targeting segment of between 21 to 72 nucleotides length, flanked by two direct repeats (DR) of a length of between 21 to 46 nucleotides (see, e.g., WO2014/131833). In the case of S. pyogenes, the DRs are 36 nucleotides long and the targeting segment is 30 nucleotides long. The 3′ located DR is complementary to and hybridizes with the corresponding tracrRNA, which in turn binds to the Cas9 protein.

Alternatively, a CRISPR system used herein can further employ a fused crRNA-tracrRNA construct (i.e., a single transcript) that functions with the codon-optimized Cas9. This single RNA is often referred to as a guide RNA or gRNA. Within a gRNA, the crRNA portion is identified as the “target sequence” for the given recognition site and the tracrRNA is often referred to as the “scaffold.” Briefly, a short DNA fragment containing the target sequence is inserted into a guide RNA expression plasmid. The gRNA expression plasmid comprises the target sequence (in some aspects around 20 nucleotides), a form of the tracrRNA sequence (the scaffold) as well as a suitable promoter that is active in the cell and necessary elements for proper processing in eukaryotic cells. Many of the systems rely on custom, complementary oligos that are annealed to form a double stranded DNA and then cloned into the gRNA expression plasmid.

The gRNA expression cassette and the Cas9 expression cassette are then introduced into the cell. See, for example, Mali P et al., (2013) Science 2013 Feb. 15; 339(6121):823-6; Jinek M et al., Science 2012 Aug. 17; 337(6096):816-21; Hwang W Y et al., Nat Biotechnol 2013 March; 31(3):227-9; Jiang W et al., Nat Biotechnol 2013 March; 31(3):233-9; and Cong L et al., Science 2013 Feb. 15; 339(6121):819-23, each of which is herein incorporated by reference in its entirety. See also, for example, WO/2013/176772 A1, WO/2014/065596 A1, WO/2014/089290 A1, WO/2014/093622 A2, WO/2014/099750 A2, and WO/2013142578 A1, each of which is herein incorporated by reference in its entirety.

### II.c.ii TALEN

In some aspects, a gene editing tool that can be used to edit (e.g., reduce or inhibit) the expression of SETD7 gene and/or SETD7 protein is a nuclease agent, such as a Transcription Activator-Like Effector Nuclease (TALEN). TAL effector nucleases are a class of sequence-specific nucleases that can be used to make double-strand breaks at specific target sequences in the genome of a prokaryotic or eukaryotic organism. TAL effector nucleases are created by fusing a native or engineered transcription activator-like (TAL) effector, or functional part thereof, to the catalytic domain of an endonuclease, such as, for example, FokI.

The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al., (2010) PNAS 10.1073/pnas.1013133107; Scholze & Boch (2010) Virulence 1:428-432; Christian et al., Genetics (2010) 186:757-761; Li et al., (2010) Nuc. Acids Res. (2010) doi:10.1093/nar/gkg704; and Miller et al., (2011) Nature Biotechnology 29:143-148; all of which are herein incorporated by reference in their entirety.

Non-limiting examples of suitable TAL nucleases, and methods for preparing suitable TAL nucleases, are disclosed, e.g., in US Patent Application No. 2011/0239315 A1, 2011/0269234 A1, 2011/0145940 A1, 2003/0232410 A1, 2005/0208489 A1, 2005/0026157 A1, 2005/0064474 A1, 2006/0188987 A1, and 2006/0063231 A1 (each hereby incorporated by reference).

In various aspects, TAL effector nucleases are engineered that cut in or near a target nucleic acid sequence in, e.g., a genomic locus of interest, wherein the target nucleic acid sequence is at or near a sequence to be modified by a targeting vector. The TAL nucleases suitable for use with the various methods and compositions provided herein include those that are specifically designed to bind at or near target nucleic acid sequences to be modified by targeting vectors as described herein.

### II.c.iii Zinc Finger Nuclease (ZFN)

In some aspects, a gene editing tool useful for the present disclosure includes a nuclease agent, such as a zinc-finger nuclease (ZFN) system. Zinc finger-based systems comprise a fusion protein comprising two protein domains: a zinc finger DNA binding domain and an enzymatic domain. A “zinc finger DNA binding domain,” “zinc finger protein,” or “ZFP” is a protein, or a domain within a larger protein, that binds DNA in a sequence-specific manner through one or more zinc fingers, which are regions of amino acid sequence within the binding domain whose structure is stabilized through coordination of a zinc ion. The zinc finger domain, by binding to a target DNA sequence (e.g., SETD7), directs the activity of the enzymatic domain to the vicinity of the sequence and, hence, induces modification of the endogenous target gene in the vicinity of the target sequence. A zinc finger domain can be engineered to bind to virtually any desired sequence. As disclosed herein, in some aspects, the zinc finger domain binds a DNA sequence that encodes the SETD7 protein. Accordingly, after identifying a target genetic locus containing a target DNA sequence at which cleavage or recombination is desired (e.g., a target locus in a target gene referenced in Table 1), one or more zinc finger binding domains can be engineered to bind to one or more target DNA sequences in the target genetic locus. Expression of a fusion protein comprising a zinc finger binding domain and an enzymatic domain in a cell, effects modification in the target genetic locus.

In some aspects, a zinc finger binding domain comprises one or more zinc fingers. Miller et al., (1985) EMBO J. 4:1609-1614; Rhodes (1993) Scientific American February:56-65; U.S. Pat. No. 6,453,242. Typically, a single zinc finger domain is about 30 amino acids in length. An individual zinc finger binds to a three-nucleotide (i.e., triplet) sequence (or a four-nucleotide sequence which can overlap, by one nucleotide, with the four-nucleotide binding site of an adjacent zinc finger). Therefore, the length of a sequence to which a zinc finger binding domain is engineered to bind (e.g., a target sequence) will determine the number of zinc fingers in an engineered zinc finger binding domain. For example, for ZFPs in which the finger motifs do not bind to overlapping subsites, a six-nucleotide target sequence is bound by a two-finger binding domain; a nine-nucleotide target sequence is bound by a three-finger binding domain, etc. Binding sites for individual zinc fingers (i.e., subsites) in a target site need not be contiguous, but can be separated by one or several nucleotides, depending on the length and nature of the amino acids sequences between the zinc fingers (i.e., the inter-finger linkers) in a multi-finger binding domain. In some aspects, the DNA-binding domains of individual ZFNs comprise between three and six individual zinc finger repeats and can each recognize between 9 and 18 basepairs.

Zinc finger binding domains can be engineered to bind to a sequence of choice. See, for example, Beerli et al., (2002) Nature Biotechnol. 20:135-141; Pabo et al., (2001) Ann. Rev. Biochem. 70:313-340; Isalan et al., (2001) Nature Biotechnol. 19:656-660; Segal et al., (2001) Curr. Opin. Biotechnol. 12:632-637; Choo et al., (2000) Curr. Opin. Struct. Biol. 10:411-416. An engineered zinc finger binding domain can have a novel binding specificity, compared to a naturally-occurring zinc finger protein. Engineering methods include, but are not limited to, rational design and various types of selection.

### II.c.iv Meganucleases

In some aspects, a gene editing tool that be used to regulate SETD7 expression in a cell includes a nuclease agent such as a meganuclease system. Meganucleases have been classified into four families based on conserved sequence motifs, the families are the “LAGLIDADG,” “GIY-YIG,” “H-N-H,” and “His-Cys box” families. These motifs participate in the coordination of metal ions and hydrolysis of phosphodiester bonds.

HEases are notable for their long recognition sites, and for tolerating some sequence polymorphisms in their DNA substrates. Meganuclease domains, structure and function are known, see, for example, Guhan and Muniyappa (2003) Crit Rev Biochem Mol Biol 38:199-248; Lucas et al., (2001) Nucleic Acids Res 29:960-9; Jurica and Stoddard, (1999) Cell Mol Life Sci 55:1304-26; Stoddard, (2006) Q Rev Biophys 38:49-95; and Moure et al., (2002) Nat Struct Biol 9:764.

In some examples a naturally occurring variant, and/or engineered derivative meganuclease is used. Methods for modifying the kinetics, cofactor interactions, expression, optimal conditions, and/or recognition site specificity, and screening for activity are known, see for example, Epinat et al., (2003) Nucleic Acids Res 31:2952-62; Chevalier et al., (2002) Mol Cell 10:895-905; Gimble et al., (2003) Mol Biol 334:993-1008; Seligman et al., (2002) Nucleic Acids Res 30:3870-9; Sussman et al., (2004) J Mol Biol 342:31-41; Rosen et al., (2006) Nucleic Acids Res 34:4791-800; Chames et al., (2005) Nucleic Acids Res 33:e178; Smith et al., (2006) Nucleic Acids Res 34:e149; Gruen et al., (2002) Nucleic Acids Res 30:e29; Chen and Zhao, (2005) Nucleic Acids Res 33:e154; WO2005105989; WO2003078619; WO2006097854; WO2006097853; WO2006097784; and WO2004031346; each of which is herein incorporated by reference in its entirety.

Any meganuclease can be used herein, including, but not limited to, I-SceI, I-SceII, I-SceIII, I-SceIV, I-SceV, I-SecVI, I-SceVII, I-CeuI, I-CeuAIIP, I-CreI, I-CrepsbIP, I-CrepsbIIP, I-CrepsbIIIP, I-CrepsbIVP, I-TliI, I-PpoI, PI-PspI, F-SceI, F-SceII, F-SuvI, F-TevI, F-TevII, I-AmaI, I-Anil, I-ChuI, I-CmoeI, I-CpaI, I-CpaII, I-CsmI, I-CvuI, I-CvuAIP, I-DdiI, I-DdiII, I-DirI, I-DmoI, I-HmuI, I-HmuII, I-HsNIP, I-LlaI, I-MsoI, I-NaaI, I-NanI, I-NcIIP, I-NgrIP, I-NitI, I-NjaI, I-Nsp236IP, I-PakI, I-PboIP, I-PcuIP, I-PcuAI, I-PcuVI, I-PgrIP, I-PobIP, I-PorIIP, I-PbpIP, I-SpBetaTP, I-Scal, I-SexIP, I-SneIP, I-SpomI, I-SpomCP, I-SpomIP, I-SpomIIP, I-SquIP, I-Ssp6803I, I-SthPhiJP, I-SthPhiST3P, I-SthPhiSTe3bP, I-TdeIP, I-TevI, I-TevII, I-TevIII, I-UarAP, I-UarHGPAIP, I-UarHGPA13P, I-VinIP, I-ZbiIP, PI-MtuI, PI-MtuHIP, PI-MtuHIIP, PI-PfuI, PI-PfuII, PI-PkoI, PI-PkoII, PI-Rma43812IP, PI-SpBetaIP, PI-SceI, PI-TfuI, PI-TfuII, PI-ThyI, PI-TliI, PI-TliII, or any active variants or fragments thereof.

### III. Fusion and Conjugates

In some aspects, the SETD7 modulator of the present disclosure (a SETD7 modulator polypeptide, e.g., a recombinant polypeptide, synthetic polypeptide, or polypeptide encoded by SETD7 modulator polynucleotide) is a fusion protein or conjugate comprising at least one heterologous moiety or an isolated polynucleotide encoding the fusion protein or conjugate.

As used herein, the term “heterologous moiety” refers to any molecule (chemical or biological), e.g., a half-life extending moiety or a detectable moiety, that is different from a SETD7 polypeptide or polynucleotide modulator disclosed herein, and which is genetically fused, conjugated, and/or otherwise associated to a SETD7 modulator of the present disclosure.

In some aspects, at least one heterologous moiety comprises a serum half-life extending moiety. The term “half-life extending moiety” refers to a pharmaceutically acceptable moiety, domain, or molecule covalently linked (“conjugated” or “fused”) to a SETD7 modulator of the present disclosure, optionally via a non-naturally encoded amino acid, directly or via a linker, that prevents or mitigates in vivo proteolytic degradation or other activity-diminishing chemical modification of the SETD7 modulator of the present disclosure, increases half-life, and/or improves or alters other pharmacokinetic or biophysical properties including but not limited to increasing the rate of absorption, reducing toxicity, improving solubility, reducing protein aggregation, increasing biological activity and/or target selectivity of the SETD7 modulator of the present disclosure, increasing manufacturability, and/or reducing immunogenicity of the SETD7 modulator of the present disclosure, compared to a reference compound such as a non-conjugated or non-fused form of the SETD7 modulator of the present disclosure.

In the context of the present disclosure, the terms “fused” or “fusion” indicate that at least two polypeptide chains have been operably linked and recombinantly expressed. In some aspects, two polypeptide chains can be “fused” as a result of chemical synthesis. In the context of the present disclosure, the terms “conjugate” or “conjugation” denote that two molecular entities (e.g., two polypeptides, or a polypeptide and a polymer such as PEG) have been chemically linked.

In some aspects, the serum half-life extending moiety comprises an Fc region, albumin, albumin binding polypeptide, a fatty acid, PAS, a glycine-rich homo-amino-acid polymer (HAP), the R subunit of the C-terminal peptide (CTP) of human chorionic gonadotropin, polyethylene glycol (PEG), hydroxyethyl starch (HES), XTEN, albumin-binding small molecules, or a combination thereof.

As used herein, the term “Fc region” is defined as the portion of a antibody which corresponds to the Fc region of the native immunoglobulin, i.e., as formed by the dimeric association of the respective Fc domains of its two heavy chains. A native Fc region forms a homodimer with another Fc region. In one aspect, the “Fc region” refers to the portion of a single immunoglobulin heavy chain beginning in the hinge region just upstream of the papain cleavage site (i.e. residue 216′ in IgG, taking the first residue of heavy chain constant region to be 114) and ending at the C-terminus of the antibody. Accordingly, a complete Fc domain comprises at least a hinge domain, a CH2 domain, and a CH3 domain. The Fc region of an immunoglobulin constant region, depending on the immunoglobulin isotype can include the CH2, CH3, and CH4 domains, as well as the hinge region. Fusion proteins comprising an Fc region of an immunoglobulin bestow several desirable properties on a fusion protein including increased stability, increased serum half-life (see Capon et al., 1989, Nature 337:525) as well as binding to Fc receptors such as the neonatal Fc receptor (FcRn) (U.S. Pat. Nos. 6,086,875, 6,485,726, 6,030,613; WO 03/077834; US2003-0235536A1).

In certain aspects, the half-life extension moiety linked, e.g., fused or conjugated, to a SETD7 modulator of the present disclosure is an albumin binding moiety, which comprises an albumin binding peptide, a bacterial albumin binding domain, an albumin-binding antibody fragment, or any combinations thereof. For example, the albumin binding protein can be a bacterial albumin binding protein, an antibody or an antibody fragment including domain antibodies (see U.S. Pat. No. 6,696,245). An albumin binding protein, for example, can be a bacterial albumin binding domain, such as the one of streptococcal protein G (Konig, T. and Skerra, A. (1998) J. Immunol. Methods 218, 73-83). Other examples of albumin binding peptides that can be used as conjugation partner are, for instance, those having a Cys-Xaa 1-Xaa 2-Xaa 3-Xaa 4-Cys consensus sequence, wherein Xaa 1 is Asp, Asn, Ser, Thr, or Trp; Xaa 2 is Asn, Gln, H is, Ile, Leu, or Lys; Xaa 3 is Ala, Asp, Phe, Trp, or Tyr; and Xaa 4 is Asp, Gly, Leu, Phe, Ser, or Thr as described in US patent application 2003/0069395 or Dennis et al. (Dennis et al. (2002) J. Biol. Chem. 277, 35035-35043).

In other aspects, the half-life extension moiety linked, e.g., fused or conjugated, to a SETD7 modulator of the present disclosure is a PAS sequence. A “PAS sequence,” as used herein, means an amino acid sequence comprising mainly alanine and serine residues or comprising mainly alanine, serine, and proline residues, the amino acid sequence forming random coil conformation under physiological conditions. Accordingly, the PAS sequence is a building block, an amino acid polymer, or a sequence cassette comprising, consisting essentially of, or consisting of alanine, serine, and proline which can be used as a part of the heterologous moiety in the fusion protein. Exemplary PAS sequences are provided, e.g., in US Pat. Publ. No. 2010/0292130 A1 and PCT Appl. Publ. No. WO 2008/155134 A1, both of which are incorporated by reference in their entireties.

In certain aspects, the half-life extension moiety linked, e.g., fused or conjugated, to a SETD7 modulator of the present disclosure is a glycine-rich homo-amino-acid polymer (HAP). The HAP sequence can comprise a repetitive sequence of glycine, which has at least 50 amino acids, at least 100 amino acids, 120 amino acids, 140 amino acids, 160 amino acids, 180 amino acids, 200 amino acids, 250 amino acids, 300 amino acids, 350 amino acids, 400 amino acids, 450 amino acids, or 500 amino acids in length. Non-limiting examples of the HAP sequence includes, but are not limited to (Gly)ₙ, (Gly₄Ser)ₙ or S(Gly₄Ser)ₙ, wherein n is 1, 2, 3, 4, 5, 6, 7, 8, 9, 10, 11, 12, 13, 14, 15, 16, 17, 18, 19, or 20. In one aspect, n is 20, 21, 22, 23, 24, 25, 26, 26, 28, 29, 30, 31, 32, 33, 34, 35, 36, 37, 38, 39, or 40. In another aspect, n is 50, 60, 70, 80, 90, 100, 110, 120, 130, 140, 150, 160, 170, 180, 190, or 200.

In other aspects, the half-life extension moiety linked, e.g., fused or conjugated, to a SETD7 modulator of the present disclosure is a soluble polymer known in the art, including, but not limited to, polyethylene glycol (PEG), ethylene glycol/propylene glycol copolymers, carboxymethylcellulose, dextran, or polyvinyl alcohol. The soluble polymer can be attached to any positions within the sequence SETD7 modulator of the present disclosure or at either or both termini. The soluble polymer can be attached at random positions within the SETD7 modulator sequence or at predetermined positions within the SETD7 modulator sequence and may include one, two, three or more attached soluble polymer moieties. In some aspects, the polymer is attached to a side chain of a naturally occurring amino acid. In other aspects, the polymer is attached to a side chain of a non-naturally encoded amino acid, e.g., a phenylalanine derivative such as para-acetyl-L-phenylalanine. The soluble polymer can be of any molecular weight, and can be branched or unbranched.

In certain aspects, the half-life extension moiety linked, e.g., fused or conjugated, to a SETD7 modulator of the present disclosure is hydroxyethyl starch (HES) or a derivative thereof. HES is a derivative of naturally occurring amylopectin and is degraded by alpha-amylase in the body. HES is a substituted derivative of the carbohydrate polymer amylopectin, which is present in corn starch at a concentration of up to 95% by weight. HES exhibits advantageous biological properties and is used as a blood volume replacement agent and in hemodilution therapy in the clinics (Sommermeyer et al., Krankenhauspharmazie, 8(8), 271-278 (1987); and Weidler et al., Arzneim.-Forschung/Drug Res., 41, 494-498 (1991)).

In certain aspects, the half-life extension moiety linked, e.g., fused or conjugated, to a SETD7 modulator of the present disclosure is a polysialic acid (PSA) or a derivative thereof. PSAs are naturally occurring unbranched polymers of sialic acid produced by certain bacterial strains and in mammals in certain cells Roth J., et al. (1993) in Polysialic Acid: From Microbes to Man, eds Roth J., Rutishauser U., Troy F. A. (Birkhauser Verlag, Basel, Switzerland), pp 335-348.

In some aspects, the half-life extension moiety linked, e.g., fused or conjugated, to a SETD7 modulator of the present disclosure is an XTEN sequence. As used here “XTEN sequence” refers to extended length polypeptides with non-naturally occurring, substantially non-repetitive sequences that are composed mainly of small hydrophilic amino acids, with the sequence having a low degree or no secondary or tertiary structure under physiologic conditions. As a fusion protein partner, XTENs can serve as a carrier, conferring certain desirable pharmacokinetic, physicochemical and pharmaceutical properties when linked to a SETD7 modulator of the present disclosure to create a fusion protein. Such desirable properties include but are not limited to enhanced pharmacokinetic parameters and solubility characteristics. Examples of XTEN sequences that can be used according to the present disclosure are disclosed in US Patent Publication Nos. 2010/0239554 A1, 2010/0323956 A1, 2011/0046060 A1, 2011/0046061 A1, 2011/0077199 A1, or 2011/0172146 A1, or International Patent Publication Nos. WO 2010091122 A1, WO 2010144502 A2, WO 2010144508 A1, WO 2011028228 A1, WO 2011028229 A1, or WO 2011028344 A2, all of which are herein incorporated by reference in their entireties.

In some aspects, the serum half-life of a SETD7 modulator polypeptide of the present disclosure comprising a half-time extending moiety at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% higher than the plasma half-life of a corresponding SETD7 modulator polypeptide without serum half-life extending moiety.

In some aspects, a SETD7 modulator polypeptide of the present disclosure comprising at least one heterologous moiety which comprises a detectable moiety, e.g., a radionuclide, a fluorescent molecule, or a contrast agent.

### IV. Polynucleotide Modifications

In some aspects, a SETD7 modulator polynucleotide of the present disclosure comprises at least one chemically modified nucleobase, sugar, backbone, or any combination thereof. Thus, a SETD7 modulator polynucleotide of the present disclosure can comprise one or more modifications. In some aspects, a SETD7 modulator polynucleotide of the present disclosure comprises at least one nucleotide analogue. In some aspects, at least one nucleotide analogue introduced by using IVT (in vitro transcription) or chemical synthesis is selected from the group consisting of a 2′-O-methoxyethyl-RNA (2′-MOE-RNA) monomer, a 2′-fluoro-DNA monomer, a 2′-O-alkyl-RNA monomer, a 2′-amino-DNA monomer, a locked nucleic acid (LNA) monomer, a cEt monomer, a cMOE monomer, a 5′-Me-LNA monomer, a 2′-(3-hydroxy)propyl-RNA monomer, an arabino nucleic acid (ANA) monomer, a 2′-fluoro-ANA monomer, an anhydrohexitol nucleic acid (HNA) monomer, an intercalating nucleic acid (INA) monomer, and a combination of two or more of said nucleotide analogues. In some aspects, the optimized nucleic acid molecule comprises at least one backbone modification, for example, a phosphorothioate internucleotide linkage.

In some aspects, a SETD7 modulator polynucleotide of the present disclosure can be chemically modified at terminal locations, for example by introducing M (2′-O-methyl), MS (2′-O-methyl 3′ phosphorothioate), or MSP (2′-O-methy 3′thioPACE, phosphonoacetate) modifications, or combinations thereof at positions 1, 2, 3 respect to the 5′ and/or 3′ termini.

Modified SETD7 modulator polynucleotides of the present disclosure need not be uniformly modified along the entire length of the molecule. Different nucleotide modifications and/or backbone structures may exist at various positions in the nucleic acid. One of ordinary skill in the art will appreciate that the nucleotide analogs or other modification(s) may be located at any position(s) of a nucleic acid such that the function of the nucleic acid is not substantially decreased. A modification may also be a 5′ or 3′ terminal modification. The nucleic acids may contain at a minimum one and at maximum 100% modified nucleotides, or any intervening percentage, such as at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% modified nucleotides.

In some aspects, a SETD7 modulator polynucleotide of the present disclosure can include modifications to prevent rapid degradation by endo- and exo-nucleases. Modifications include, but are not limited to, for example, (a) end modifications, e.g., 5′ end modifications (phosphorylation dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages.

Specific examples of synthetic, modified SETD7 modulator polynucleotides of the present disclosure useful with the methods described herein include, but are not limited to, a SETD7 modulator polynucleotides containing modified or non-natural internucleoside linkages. Synthetic, modified SETD7 modulator polynucleotides having modified internucleoside linkages include, among others, those that do not have a phosphorus atom in the internucleoside linkage. In other embodiments, the synthetic, modified SETD7 modulator polynucleotides has a phosphorus atom in its internucleoside linkage(s).

Non-limiting examples of modified internucleoside linkages include phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, T-5′ linked analogs of these, and those) having inverted polarity wherein the adjacent pairs of nucleoside units are linked 3′-5′ to 5′-3′ or T-5′ to 5′-T. Various salts, mixed salts and free acid forms are also included.

Modified internucleoside linkages that do not include a phosphorus atom therein have internucleoside linkages that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts.

In some aspects, a SETD7 modulator polynucleotide of the present disclosure can be codon optimized by introducing one or more synonymous codon changes. As used herein, the terms “codon optimization,” “codon optimized,” and grammatical variants thereof refer to the modification of the primary sequence of a nucleic acid by replacing synonymous codons in order to increase its translational efficiency. Accordingly, codon optimization comprises switching the codons used in a SETD7 modulator polynucleotide of the present disclosure without changing the amino acid sequence that it encodes for, which typically dramatically increases the abundance of the protein the codon optimized gene encodes because it generally removes “rare” codons and replaces them with abundant codons, or removes codon with a low tRNA recharge rate with codon with high tRNA recharge rates. Such codon optimization can, for example, (i) improve protein yield in recombinant protein expression, or (ii) improve the stability, half life, or other desirable property of an mRNA or a DNA encoding a binding molecule disclosed herein, wherein such mRNA or DNA is administered to a subject in need thereof.

SETD7 modulator polynucleotide sequences of the present disclosure can be codon optimized using any methods known in the art at the time the present application was filed.

In some aspects, a SETD7 modulator polynucleotide encoding a SETD7 modulator polypeptide has been sequence optimized. As used herein, the term “sequence optimized” refers to the modification of the sequence of a nucleic acid by to introduce features that increase its translational efficiency, remove features that reduce its translational efficiency, or in general improve properties related to expression efficacy after administration in vivo. Such properties include, but are not limited to, improving nucleic acid stability (e.g., mRNA stability), increasing translation efficacy in the target tissue, reducing the number of truncated proteins expressed, improving the folding or prevent misfolding of the expressed proteins, reducing toxicity of the expressed products, reducing cell death caused by the expressed products, or increasing and/or decreasing protein aggregation

### V. Vectors and Cells

The present disclosure also provides a vector comprising an isolated polynucleotide, e.g., a SETD7 modulator polynucleotide disclosed herein. In some aspects, the SETD7 modulator is a polypeptide encoded by any of the polynucleotides disclosed herein, e.g., an isolated polynucleotide (e.g., a SETD7 modulator polynucleotide encoding a SETD7 polypeptide) or a vector (e.g., a vector comprising a SETD7 modulator polynucleotide encoding a SETD7 polypeptide).

In some aspects, the vector is viral vector. In some aspects, the viral vector is an adenoviral vector or an adenoassociated viral vector. In some aspects, the adenoviral vector is a third generation adenoviral vector. ADEASY™ is by far the most popular method for creating adenoviral vector constructs. The system consists of two types of plasmids: shuttle (or transfer) vectors and adenoviral vectors. The transgene of interest is cloned into the shuttle vector, verified, and linearized with the restriction enzyme PmeI. This construct is then transformed into ADEASIER-1 cells, which are BJ5183 E. coli cells containing PADEASY™. PADEASY™ is a ˜33 Kb adenoviral plasmid containing the adenoviral genes necessary for virus production. The shuttle vector and the adenoviral plasmid have matching left and right homology arms which facilitate homologous recombination of the transgene into the adenoviral plasmid. One can also co-transform standard BJ5183 with supercoiled PADEASY™ and the shuttle vector, but this method results in a higher background of non-recombinant adenoviral plasmids. Recombinant adenoviral plasmids are then verified for size and proper restriction digest patterns to determine that the transgene has been inserted into the adenoviral plasmid, and that other patterns of recombination have not occurred. Once verified, the recombinant plasmid is linearized with PacI to create a linear dsDNA construct flanked by ITRs. 293 or 911 cells are transfected with the linearized construct, and virus can be harvested about 7-10 days later. In addition to this method, other methods for creating adenoviral vector constructs known in the art at the time the present application was filed can be used to practice the methods disclosed herein.

In other aspects, the viral vector is a retroviral vector, e.g., a lentiviral vector (e.g., a third or fourth generation lentiviral vector). Lentiviral vectors are usually created in a transient transfection system in which a cell line is transfected with three separate plasmid expression systems. These include the transfer vector plasmid (portions of the HIV provirus), the packaging plasmid or construct, and a plasmid with the heterologous envelop gene (env) of a different virus. The three plasmid components of the vector are put into a packaging cell which is then inserted into the HIV shell. The virus portions of the vector contain insert sequences so that the virus cannot replicate inside the cell system. Current third generation lentiviral vectors encode only three of the nine HIV-1 proteins (Gag, Pol, Rev), which are expressed from separate plasmids to avoid recombination-mediated generation of a replication-competent virus. In fourth generation lentiviral vectors, the retroviral genome has been further reduced (see, e.g., TAKARA® LENTI-X™ fourth-generation packaging systems).

In some aspects, a nucleic acid sequence comprising a SETD7 modulator polynucleotide, e.g., a SETD7 modulator polynucleotide encoding a SETD7 modulator polypeptide, can be inserted into the genome of a target cell (e.g., pancreatic cell) or a host cell (e.g., a stem cell for transplantation to the target tissue) by using CRISPR/Cas systems and genome edition alternatives such as zinc-finger nucleases (ZFNs), transcription activator-like effector nucleases (TALENs), and meganucleases (MNs).

In some aspects, the polynucleotide of the present disclosure comprises a virus, a plasmid, or a phagemid encoding a SETD7 modulator. In some aspects, the virus is selected from the group consisting of an adeno-associated virus (AAV), a retrovirus, a lentivirus, an adenovirus, an SV40-type virus, a polyomavirus, an Epstein-Barr virus, a papilloma viruses, a herpes virus, a vaccinia virus, a polio virus, and an RNA virus.

Any AAV vector known in the art can be used in the methods disclosed herein. The AAV vector can comprise a known vector or can comprise a variant, fragment, or fusion thereof. In some embodiments, the AAV vector is selected from the group consisting of AAV type 1 (AAV1), AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, and any combination thereof.

In some embodiments, the AAV vector is derived from an AAV vector selected from the group consisting of AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, and any combination thereof.

In some embodiments, the AAV vector is a chimeric vector derived from at least two AAV vectors selected from the group consisting of AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, and any combination thereof.

In some aspects, the AAV vector comprises regions of at least two different AAV vectors known in the art.

In some aspects, the AVV vector comprises a portion of an AAV vector selected from the group consisting of AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, and any combination thereof.

In some aspects, the AVV vector comprises a splice acceptor site. In some aspects, the AVV vector comprises a promoter. Any promoter known in the art can be used in the AAV vector of the present disclosure. In some aspects, the AAV vector comprises a constitutively active promoter (constitutive promoter). In some aspects, the promoter is an inducible promoter. In some aspects, the inducible promoter is a tissue specific promoter.

In some aspects, the AVV vector comprises one or more enhancers. In some aspects, the one or more enhancer are present in the AAV alone or together with a promoter disclosed herein. In some embodiments, the AAV vector comprises a 3′UTR poly(A) tail sequence. In some embodiments, the 3′UTR poly(A) tail sequence is selected from the group consisting of bGH poly(A), actin poly(A), hemoglobin poly(A), and any combination thereof. In some embodiments, the 3′UTR poly(A) tail sequence comprises bGH poly(A).

Thus, the present disclosure also provides cells comprising a SETD7 modulator polynucleotide, e.g., a SETD7 modulator polynucleotide encoding a SETD7 modulator polypeptide disclosed herein, or a vector comprising a SETD7 modulator polynucleotide.

In some aspects, a SETD7 modulator of the present disclosure, e.g., a SETD7 modulator polynucleotide such as a SETD7 modulator polynucleotide encoding a SETD7 modulator polypeptide disclosed herein, or a vector comprising a SETD7 modulator polynucleotide, can be incorporated into a cell in vivo, in vitro, or ex vivo

### VI. Delivery Systems

In some aspects, a SETD7 modulator of the present disclosure (e.g., a SETD7 modulator polypeptide, a SETD7 modulator polynucleotide, or a vector comprising a SETD7 modulator polynucleotide encoding a SETD7 modulator polypeptide) can be administered with a delivery agent, e.g., a lipidoid, a liposome, a lipoplex, a lipid nanoparticle, a polymeric compound, a peptide, a protein, a cell, a nanoparticle mimic, a nanotube, a micelle, or a conjugate.

Thus, the present disclosure also provides is a composition comprising a SETD7 modulator of the present disclosure (e.g., a SETD7 modulator polypeptide, a SETD7 modulator polynucleotide, or a vector comprising a SETD7 modulator polynucleotide encoding a SETD7 modulator polypeptide) and a delivery agent, e.g., a lipidoid, a liposome, a lipoplex, a lipid nanoparticle, a polymeric compound, a peptide, a protein, a cell, a nanoparticle mimic, a nanotube, a micelle, or a conjugate. In some aspects, the delivery agent comprises a cationic carrier unit comprising

[WP]-L1-[CC]-L2-[AM] (formula I)

or

[WP]-L1-[AM]-L2-[CC] (formula II)

wherein
WP is a water-soluble biopolymer moiety;
CC is a positively charged carrier moiety;
AM is an adjuvant moiety; and,
L1 and L2 are independently optional linkers, and
wherein when mixed with a nucleic acid at an ionic ratio of about 1:1, the cationic carrier unit forms a micelle.

In some aspects, composition comprising a SETD7 modulator of the present disclosure (e.g., a SETD7 modulator polypeptide, a SETD7 modulator polynucleotide, or a vector comprising a SETD7 modulator polynucleotide encoding a SETD7 modulator polypeptide) interacts with the cationic carrier unit via an ionic bond.

In some aspects, the water-soluble polymer comprises poly(alkylene glycols), poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyglycerol, polyphosphazene, polyoxazolines (“POZ”) poly(N-acryloylmorpholine), or any combinations thereof. In some aspects, the water-soluble polymer comprises polyethylene glycol (“PEG”), polyglycerol, or poly(propylene glycol) (“PPG”). In some aspects, the water-soluble polymer comprises:

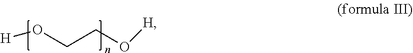

wherein n is 1-1000.

In some aspects, the n is at least about 110, at least about 111, at least about 112, at least about 113, at least about 114, at least about 115, at least about 116, at least about 117, at least about 118, at least about 119, at least about 120, at least about 121, at least about 122 at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, or at least about 141. In some aspects, the n is about 80 to about 90, about 90 to about 100, about 100 to about 110, about 110 to about 120, about 120 to about 130, about 140 to about 150, about 150 to about 160.

In some aspects, the water-soluble polymer is linear, branched, or dendritic. In some aspects, the cationic carrier moiety comprises one or more basic amino acids. In some aspects, the cationic carrier moiety comprises at least three, at least four, at least five, at least six, at least seven, at least eight, at least nine, at least ten, at least 11, at least 12, at least 13, at least 14, at last 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 basic amino acids. In some aspects, the cationic carrier moiety comprises about 30 to about 50 basic amino acids. In some aspects, the basic amino acid comprises arginine, lysine, histidine, or any combination thereof. In some aspects, the cationic carrier moiety comprises about 40 lysine monomers.

In some aspects, the adjuvant moiety is capable of modulating an immune response, an inflammatory response, and/or a tissue microenvironment. In some aspects, the adjuvant moiety comprises an imidazole derivative, an amino acid, a vitamin, or any combination thereof. In some aspects, the adjuvant moiety comprises:

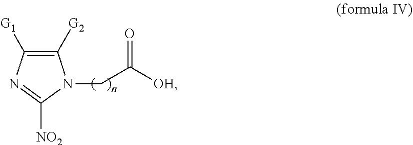

wherein each of G1 and G2 is H, an aromatic ring, or 1-10 alkyl, or G1 and G2 together form an aromatic ring, and wherein n is 1-10.

In some aspects, the adjuvant moiety comprises nitroimidazole. In some aspects, the adjuvant moiety comprises metronidazole, tinidazole, nimorazole, dimetridazole, pretomanid, ornidazole, megazol, azanidazole, benznidazole, or any combination thereof. In some aspects, the adjuvant moiety comprises an amino acid.

In some aspects, the adjuvant moiety comprises

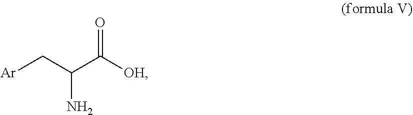

wherein Ar is

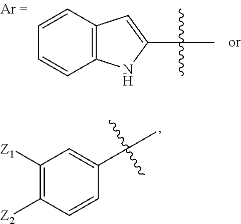

and
wherein each of Z1 and Z2 is H or OH.

In some aspects, the adjuvant moiety comprises a vitamin. In some aspects, the vitamin comprises a cyclic ring or cyclic hetero atom ring and a carboxyl group or hydroxyl group. In some aspects, the vitamin comprises:

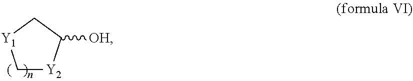

wherein each of Y1 and Y2 is C, N, O, or S, and wherein n is 1 or 2.

In some aspects, the vitamin is selected from the group consisting of vitamin A, vitamin B1, vitamin B2, vitamin B3, vitamin B6, vitamin B7, vitamin B9, vitamin B12, vitamin C, vitamin D2, vitamin D3, vitamin E, vitamin M, vitamin H, and any combination thereof. In some aspects, the vitamin is vitamin B3.

In some aspects, the adjuvant moiety comprises at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, or at least about 20 vitamin B3. In some aspects, the adjuvant moiety comprises about 10 vitamin B3.

In some aspects, the composition comprises about a water-soluble biopolymer moiety with about 120 to about 130 PEG units, a cationic carrier moiety comprising a poly-lysine with about 30 to about 40 lysines, and an adjuvant moiety with about 5 to about 10 vitamin B3.

The present disclosure also provides a micelle comprising a SETD7 modulator of the present disclosure (e.g., a SETD7 modulator polypeptide, a SETD7 modulator polynucleotide, or a vector comprising a SETD7 modulator polynucleotide encoding a SETD7 modulator polypeptide) wherein the SETD7 modulator of the present disclosure (e.g., a SETD7 modulator polypeptide, a SETD7 modulator polynucleotide, or a vector comprising a SETD7 modulator polynucleotide encoding a SETD7 modulator polypeptide), and the delivery agent are associated with each other.

In some aspects, the association is a covalent bond, a non-covalent bond, or an ionic bond. In some aspects, the positive charge of the cationic carrier moiety of the cationic carrier unit is sufficient to form a micelle when mixed with the polynucleotide, vector, or polypeptide disclosed herein in a solution, wherein the overall ionic ratio of the positive charges of the cationic carrier moiety of the cationic carrier unit and the negative charges of the polynucleotide, vector, or polypeptide in the solution is about 1:1.

In some aspects, the cationic carrier unit is capable of protecting the SETD7 modulator of the present disclosure (e.g., a SETD7 modulator polypeptide, a SETD7 modulator polynucleotide, or a vector comprising a SETD7 modulator polynucleotide encoding a SETD7 modulator polypeptide) from enzymatic degradation. See U.S. Prov. Appl. 62/867,097, which is herein incorporated by reference in its entirety.

The present disclosure also provides a SETD7 modulator (e.g., a SETD7 modulator polypeptide; a SETD7 modulator polynucleotide; a vector comprising a SETD7 modulator polynucleotide encoding a SETD7 modulator polypeptide; or a cell, micelle, or pharmaceutical compositions disclosed herein) for use as a medicament. Also provided is a SETD7 modulator (e.g., a SETD7 modulator polypeptide; a SETD7 modulator polynucleotide; a vector comprising a SETD7 modulator polynucleotide encoding a SETD7 modulator polypeptide; or a cell, micelle, or pharmaceutical compositions disclosed herein) for use in the treatment of a metabolic disease in a subject in need thereof, e.g., diabetes. Also provided is a SETD7 modulator (e.g., a SETD7 modulator polypeptide; a SETD7 modulator polynucleotide; a vector comprising a SETD7 modulator polynucleotide encoding a SETD7 modulator polypeptide; or a cell, micelle, or pharmaceutical compositions disclosed herein) for use in the treatment of cancer.

The present disclosure also provides the use of a SETD7 modulator (e.g., a SETD7 modulator polypeptide; a SETD7 modulator polynucleotide; a vector comprising a SETD7 modulator polynucleotide encoding a SETD7 modulator polypeptide; or a cell, micelle, or pharmaceutical compositions disclosed herein) in the manufacture of treatment for a metabolic disease, e.g., diabetes. The present disclosure also provides the use of a SETD7 modulator (e.g., a SETD7 modulator polypeptide; a SETD7 modulator polynucleotide; a vector comprising a SETD7 modulator polynucleotide encoding a SETD7 modulator polypeptide; or a cell, micelle, or pharmaceutical compositions disclosed herein) in the manufacture of treatment for cancer.

### VII. Pharmaceutical Compositions

The present disclosure also provides pharmaceutical compositions comprising SETD7 modulators of the present disclosure (e.g., SETD7 modulator polypeptides; SETD7 modulator polynucleotides; vectors comprising a SETD7 modulator polynucleotide encoding a SETD7 modulator polypeptide; or cells or micelles disclosed herein) that are suitable for administration to a subject. The pharmaceutical compositions generally comprise a SETD7 modulator of the present disclosure (e.g., a SETD7 modulator polypeptide; a SETD7 modulator polynucleotide; a vectors comprising a SETD7 modulator polynucleotide encoding a SETD7 modulator polypeptide; or a cell or micelle disclosed herein) and a pharmaceutically-acceptable excipient or carrier in a form suitable for administration to a subject. Pharmaceutically acceptable excipients or carriers are determined in part by the particular composition being administered, as well as by the particular method used to administer the composition.

There is a wide variety of suitable formulations of pharmaceutical compositions comprising a SETD7 modulator of the present disclosure (see, e.g., Remington's Pharmaceutical Sciences, Mack Publishing Co., Easton, Pa. 18th ed. (1990)). The pharmaceutical compositions are generally formulated sterile and in full compliance with all Good Manufacturing Practice (GMP) regulations of the U.S. Food and Drug Administration. In some aspects, the pharmaceutical composition comprises one or more SETD7 modulators of the present disclosure.

In certain aspects, the SETD7 modulators of the present disclosure are co-administered with of one or more additional therapeutic agents, in a pharmaceutically acceptable carrier. In some aspects, the pharmaceutical composition comprising the SETD7 modulators of the present disclosure is administered prior to administration of the additional therapeutic agent(s). In other aspects, the pharmaceutical composition comprising the SETD7 modulators of the present disclosure is administered after the administration of the additional therapeutic agent(s). In further aspects, the pharmaceutical composition comprising the SETD7 modulator of the present disclosure is administered concurrently with the additional therapeutic agent(s).

Acceptable carriers, excipients, or stabilizers are nontoxic to recipients (e.g., animals or humans) at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Examples of carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the SETD7 modulators of the present disclosure, use thereof in the compositions is contemplated.

Supplementary therapeutic agents can also be incorporated into the compositions of the present disclosure. Typically, a pharmaceutical composition is formulated to be compatible with its intended route of administration. The SETD7 modulators of the present disclosure can be administered by parenteral, topical, intravenous, oral, subcutaneous, intra-arterial, intradermal, transdermal, rectal, intracranial, intraperitoneal, intranasal, intratumoral, intramuscular route or as inhalants. In certain aspects, the SETD7 modulators of the present disclosure are administered intravenously, e.g. by injection. The SETD7 modulators of the present disclosure can optionally be administered in combination with other therapeutic agents that are at least partly effective in treating the disease, disorder or condition for which the SETD7 modulators of the present disclosure are intended, e.g., for the treatment of diabetes or cancer.

Solutions or suspensions can include the following components: a sterile diluent such as water, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The preparation can be enclosed in ampoules, disposable syringes or multiple dose vials made of glass or plastic.

Pharmaceutical compositions suitable for injectable use include sterile aqueous solutions (if water soluble) or dispersions and sterile powders. For intravenous administration, suitable carriers include physiological saline, bacteriostatic water, Cremophor EL™ (BASF, Parsippany, N.J.) or phosphate buffered saline (PBS). The composition is generally sterile and fluid to the extent that easy syringeability exists. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. If desired, isotonic compounds, e.g., sugars, polyalcohols such as mannitol, sorbitol, and sodium chloride can be added to the composition. Prolonged absorption of the injectable compositions can be brought about by including in the composition a compound which delays absorption, e.g., aluminum monostearate and gelatin.

Pharmaceutical compositions of the present disclosure can be sterilized by conventional, well known sterilization techniques. Aqueous solutions can be packaged for use or filtered under aseptic conditions and lyophilized, the lyophilized preparation being combined with a sterile aqueous solution prior to administration.

Sterile injectable solutions can be prepared by incorporating the SETD7 modulators of the present disclosure in an effective amount and in an appropriate solvent with one or a combination of ingredients enumerated herein, as desired. Generally, dispersions are prepared by incorporating the SETD7 modulators of the present disclosure into a sterile vehicle that contains a basic dispersion medium and any desired other ingredients. In the case of sterile powders for the preparation of sterile injectable solutions, methods of preparation are vacuum drying and freeze-drying that yields a powder of the active ingredient plus any additional desired ingredient from a previously sterile-filtered solution thereof. The SETD7 modulators of the present disclosure can be administered in the form of a depot injection or implant preparation which can be formulated in such a manner to permit a sustained or pulsatile release of the SETD7 modulators of the present disclosure.

Systemic administration of pharmaceutical compositions comprising the SETD7 modulators of the present disclosure can also be by transmucosal means. For transmucosal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of, e.g., nasal sprays.

In certain aspects, pharmaceutical compositions comprising the SETD7 modulators of the present disclosure can be administered intravenously into a subject that would benefit from the pharmaceutical composition. In certain other aspects, the composition is administered to the lymphatic system, e.g., by intralymphatic injection or by intranodal injection (see e.g., Senti et al., PNAS 105(46): 17908 (2008)), or by intramuscular injection, by subcutaneous administration, by intratumoral injection, by direct injection into the thymus, or into the liver.

In certain aspects, the pharmaceutical composition comprising the SETD7 modulators of the present disclosure is administered as a liquid suspension. In certain aspects, the pharmaceutical composition is administered as a formulation that is capable of forming a depot following administration. In certain preferred aspects, the depot slowly releases the micelles described herein into circulation, or remains in depot form.

Typically, pharmaceutically-acceptable compositions are highly purified to be free of contaminants, are biocompatible and not toxic, and are suited to administration to a subject. If water is a constituent of the carrier, the water is highly purified and processed to be free of contaminants, e.g., endotoxins.

The pharmaceutically-acceptable carrier can be lactose, dextrose, sucrose, sorbitol, mannitol, starch, gum acacia, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and/or mineral oil, but is not limited thereto. The pharmaceutical composition can further include a lubricant, a wetting agent, a sweetener, a flavor enhancer, an emulsifying agent, a suspension agent, and/or a preservative.

The pharmaceutical compositions described herein can comprise micelles comprising SETD7 modulators of the present disclosure and optionally a pharmaceutically active or therapeutic agent. The therapeutic agent can be a biological agent, a small molecule agent, or a nucleic acid agent.

Dosage forms are provided that comprise SETD7 modulators of the present disclosure. In some aspects, the dosage form is formulated as a liquid suspension for intravenous injection. Actual dosage levels of SETD7 modulators of the present disclosure can be varied so as to obtain an amount of the active ingredient which is effective to achieve the desired therapeutic response for a particular subject, composition, and mode of administration, without being toxic to the subject. The selected dosage level depends upon a variety of pharmacokinetic factors including the activity of the particular therapeutic agent, the route of administration, the time of administration, the rate of excretion of the particular compound being employed, the severity of the condition, other health considerations affecting the subject, and the status of liver and kidney function of the subject. It also depends on the response to administration of the agents, including factors such as blood sugar level or the level of glycated hemoglobin, body mass index as well as levels of enzymes involved in liver disease such as ALT and AST. It also depends on the duration of the treatment, other drugs, compounds and/or materials used in combination with the particular therapeutic agent employed, as well as the age, weight, condition, general health and prior medical history of the subject being treated, and like factors. Methods for determining optimal dosages are described in the art, e.g., Remington: The Science and Practice of Pharmacy, Mack Publishing Co., 20th ed., 2000.

The SETD7 modulators of the present disclosure (e.g., SETD7 modulator polypeptides; SETD7 modulator polynucleotides; vectors comprising a SETD7 modulator polynucleotide encoding a SETD7 modulator polypeptide; cells; micelles; or pharmaceutical compositions disclosed herein) may be used concurrently with other drugs. To be specific, the micelles or pharmaceutical compositions of the present disclosure may be used together with medicaments such as hormonal therapeutic agents, chemotherapeutic agents, immunotherapeutic agents, medicaments inhibiting the action of cell growth factors or cell growth factor receptors and the like.

In some aspects, SETD7 modulators of the present disclosure (e.g., SETD7 modulator polypeptides; SETD7 modulator polynucleotides; vectors comprising a SETD7 modulator polynucleotide encoding a SETD7 modulator polypeptide; cells; micelles; or pharmaceutical compositions disclosed herein) can be administered in combination with one or more therapeutic agents used to treat type 2 diabetes disclosed herein. One commonly used class of therapeutic agents used to treat type 2 diabetes is the biguanides. The prototype of this class, and still one of the most commonly used agents for the treatment of diabetes, is metformin. Other related biguanide drugs such as phenformin or buformin have been used, but have been withdrawn due to significant side effects. Another class of therapeutic agents is the sulfonylureas. This class of agents includes acetohexamide, carbutamide, chlorpropamide, glycyclamide, metahexamide, tolazamide, tolbutamide, glibenclamide, glibomuride, gliclazide, glipizide, gliquidone, glisoxepide, glyclopyramide, and glimepiride. Although these agents may be effective in many cases of type 2 diabetes, they are associated with weight gain and may induce hypoglycemia, which can be severe. Yet another class of antidiabetic agents is the thiazolidinediones, including pioglitazone and rosiglitazone. These agents are PPAR activators and act to decrease insulin resistance and to increase storage of fatty acids, forcing cells to utilize carbohydrates for oxidation. Still another class of antidiabetic agents is the DPP-4 inhibitors. These agents are inhibitors of dipeptidyl peptidase-4. These agents act to lower the levels of glucagon and reduce blood sugar levels. They also act to increase incretin levels, which act to promote insulin release. These agents include sitagliptin, vildagliptin, saxagliptin, linagliptin, gemigliptin, anagliptin, teneligliptin, alogliptin, trelagliptin, and omarigliptin. Another class of antidiabetic agents is the gliflozins. These agents act by inhibiting sodium-glucose transport protein 2 (SGLT2) and inhibit reabsorption of glucose in the kidney, thereby lowering blood sugar. These agents include canagliflozin, dapagliflozin, and empagliflozin. Still another class of antidiabetic agents is the glucagon-like peptide-receptor agonists. These drugs act by increasing secretion of insulin. These agents include exatenide, liraglutide, lixisenatide, albiglutide, and dulaglutide. Another class of antidiabetic agents are the amylin analogs, including pramlintide.

In some aspects, a SETD7 modulator of the present disclosure (e.g., a SETD7 modulator polypeptides or a SETD7 modulator polynucleotide) is a prodrug. The term “prodrug” is also meant to include any covalently bonded carriers which release the active compound in vivo when the prodrug is administered to a subject. Prodrugs of a therapeutically active compound, as described herein, can be prepared by modifying one or more functional groups present in the therapeutically active compound in such a way that the modifications are cleaved, either in routine manipulation or in vivo, to yield the parent therapeutically active compound. The use of prodrug systems is described in T. Jarvinen et al., “Design and Pharmaceutical Applications of Prodrugs” in Drug Discovery Handbook (S. C. Gad, ed., Wiley-Interscience, Hoboken, N.J., 2005), ch. 17, pp. 733-796, incorporated herein by this reference.

### VIII. Indications

The SETD7 modulators of the present disclosure (e.g., SETD7 modulator polypeptides; SETD7 modulator polynucleotides; vectors comprising a SETD7 modulator polynucleotide encoding a SETD7 modulator polypeptide; cells; micelles; or pharmaceutical compositions disclosed herein) or combinations can be administered to a subject, e.g., a human subject, to treat diseases or conditions disclosed herein, such as diabetes (e.g., type 2 diabetes) or cancer.

As used herein, the term “diabetes,” without further limitation, refers to type 2 diabetes. However, certain methods and compositions according to the present disclosure can be useful for the treatment of type 1 diabetes or prediabetes or obesity or chronic liver disease as well. The recitation of “type 2 diabetes” in the present application is not to be interpreted to mean that any method or composition recited in the present application is not useful for the treatment of type 1 diabetes or prediabetes or obesity or chronic liver disease.

As used herein, the term “obesity” refers to a body mass index (BMI) of greater than 30. In general, a BMI from 30.0 to 35.0 is defined as class I obesity. A BMI from 35.0 to 40.0 is defined as class II obesity. A BMI of over 40.0 is defined as class III obesity. Obesity I associated with an increased risk of cardiovascular disease, hypertension, type 2 diabetes, sleep apnea, certain types of cancer, osteoarthritis and asthma, and may aggravate musculoskeletal conditions such as those resulting in back pain.

Type 2 diabetes is a metabolic disorder in which cellular uptake of glucose is impaired that causes blood glucose levels to rise higher than normal. In Type 2 diabetes, this is typically caused by the body not being able to utilize insulin properly; this is called insulin resistance. Long term complications for type 2 diabetes can include, but are not limited to, renal failure, peripheral neuropathy, diabetic retinopathy, cardiovascular complications, circulatory disorders, and reduced resistance to infections. Additionally, there is increasing evidence that type 2 diabetes can be associated with an increased frequency of some forms of Alzheimer's disease. Evidence suggests that some forms of Alzheimer's disease are triggered by insulin resistance in the brain. This condition is most often used to describe people who have type 2 diabetes and are also diagnosed with Alzheimer's or dementia, and has been termed “type 3 diabetes.”

Chronic liver disease, such as Nonalcoholic Fatty Liver Disease (NALFD) has been associated with prediabetes and type 2 diabetes and may contribute to elevated blood glucose levels and insulin resistance observed in these disorders. Chronic liver disease can lead to the development of nonalcoholic steatohepatitis, cirrhosis or liver cancer and their related complications. Small molecules that target epigenetic enzymes that can reverse chronic liver disease such as NALFD and that can decrease levels of liver enzymes such as ALT and AST would be of therapeutic advantage. There is also a strong association between type 2 diabetes and obesity.

Not only is type 2 diabetes far more common in obese individuals than those of normal weight, individuals with type 2 diabetes who were previously obese but who manage to lose enough weight so that they are no longer considered obese have a far better prognosis.

In some aspects, SETD7 modulators of the present disclosure can be used to prevent symptoms or sequelae of diseases and conditions disclosed herein. For example, treatment of type 2 diabetes can include any improvement or reduction of progression of type 2 diabetes, or, where appropriate, type 1 diabetes or prediabetes or obesity or chronic liver disease, which can be evaluated by one or more of the following criteria: reduction in blood glucose; reduction in glycated hemoglobin; improvement in response to glucose tolerance test; reduction in urinary frequency, urinary urgency, or excessive thirst; reduction in pain associated with peripheral neuropathy; improvement in wound healing, improvement in fatigue, body mass index, liver enzyme levels or any other sign or symptom associated with type 2 diabetes. The terms “treating,” treatment” or similar terminology are not intended to imply a permanent cure for type 2 diabetes, or, where appropriate, type 1 diabetes or prediabetes or chronic liver disease.

### IX. Methods of Treatment and Use

The present disclosure also provides methods of treating a disease or condition in a subject in need thereof comprising administering a SETD7 modulator of the present disclosure or a combination thereof to a subject, e.g., a mammal, such as a human subject.

Also provided are methods for treating a metabolic disease or disorder in a subject in need thereof comprising administering to the subject a therapeutically effective amount of a SETD7 modulator of the present disclosure (e.g., a polypeptide, polynucleotide, vector, composition, micelle, or pharmaceutical composition disclosed herein) or a combination thereof. In some aspects, the metabolic disease or disorder is diabetes, obesity, or insulin resistance. In some aspects, the diabetes is type 2 diabetes mellitus.

The present disclosure also provides methods for treating a cancer in a subject in need thereof comprising administering to the subject a therapeutically effective amount of a SETD7 modulator of the present disclosure (e.g., a polypeptide, polynucleotide, vector, composition, micelle, or pharmaceutical composition disclosed herein) or a combination thereof.

In some aspects, the SETD7 modulators of the present disclosure can be administered via intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

In some aspects, the SETD7 modulators of the present disclosure can be used concurrently with other medicaments or treatment suitable for the treatment of the diseases and conditions disclosed herein.

The present disclosure also provides methods for preventing or reducing (i.e., decreasing) nuclear translocation of SETD7 in a cell comprising contacting the cell with an effective amount of a SETD7 modulators of the present disclosure (e.g., a polypeptide, polynucleotide, vector, composition, micelle, or pharmaceutical composition disclosed herein).

In some aspects, contacting the cell with an effective amount of a SETD7 modulator of the present disclosure (e.g., a polypeptide, polynucleotide, vector, composition, micelle, or pharmaceutical composition disclosed herein) can decrease nuclear translocation of SETD7 by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to the level of nuclear translocation of SETD7 observed under control conditions (e.g., without contacting the cell with an effective amount of a SETD7 modulator of the present disclosure).

Also provided is a method for preventing or reducing (i.e. decreasing) nuclear accumulation of SETD7 in a cell comprising contacting the cell with an effective amount of a polynucleotide, vector, composition, micelle, or pharmaceutical composition disclosed herein.

In some aspects, contacting the cell with an effective amount of a SETD7 modulator of the present disclosure (e.g., a polypeptide, polynucleotide, vector, composition, micelle, or pharmaceutical composition disclosed herein) can decrease nuclear accumulation of SETD7 by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to the level of nuclear accumulation of SETD7 observed under control conditions (e.g., without contacting the cell with an effective amount of a SETD7 modulator of the present disclosure).

The present disclosure also provides method for preventing or reducing (i.e., decreasing) histone H3K4 monomethylation in a cell comprising contacting the cell with an effective amount of a SETD7 modulator of the present disclosure (e.g., a polypeptide, polynucleotide, vector, composition, micelle, or pharmaceutical composition disclosed herein).

In some aspects, contacting the cell with an effective amount of a SETD7 modulator of the present disclosure (e.g., a polypeptide, polynucleotide, vector, composition, micelle, or pharmaceutical composition disclosed herein) can decrease histone H3K4 monomethylation by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to the level of histone H3K4 monomethylation observed under control conditions (e.g., without contacting the cell with an effective amount of a SETD7 modulator of the present disclosure).

Also provided is a method for preventing or reducing (i.e., decreasing) p53 monomethylation in a cell comprising contacting the cell with an effective amount of a SETD7 modulator of the present disclosure (e.g., a polypeptide, polynucleotide, vector, composition, micelle, or pharmaceutical composition disclosed herein).

In some aspects, contacting the cell with an effective amount of a SETD7 modulator of the present disclosure (e.g., a polypeptide, polynucleotide, vector, composition, micelle, or pharmaceutical composition disclosed herein) can decrease p53 monomethylation by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to the level of p53 monomethylation observed under control conditions (e.g., without contacting the cell with an effective amount of a SETD7 modulator of the present disclosure).

### X. Kits

The present disclosure also provides kits or products of manufacture, comprising a SETD7 modulator of the present disclosure (e.g., a polynucleotide, vector, composition, cell, micelle, or pharmaceutical composition disclosed herein) and optionally instructions for use, e.g., instructions for use according to the methods disclosed herein.

In some aspects, the kit comprises a cationic carrier unit, a micelle, or a pharmaceutical composition comprising a SETD7 modulator of the present disclosure. In some aspects, the kit or product of manufacture comprises a SETD7 modulator of the present disclosure and a cationic carrier unit or a micelle disclosed herein in one or more containers.

In some aspects, the kit or product of manufacture comprises a SETD7 modulator of the present disclosure, optionally a cationic carrier unit or a micelle disclosed herein, and optionally a brochure. In some aspects, the kit or product of manufacture comprises a SETD7 modulator of the present disclosure, optionally a cationic carrier unit or a micelle disclosed herein, and optionally instructions for use. One skilled in the art will readily recognize that a SETD7 modulator of the present disclosure can be readily incorporated into one of the established kit formats which are well known in the art.

In some aspects, the kit or product of manufacture comprises a SETD7 modulator of the present disclosure, optionally a cationic carrier unit or a micelle disclosed herein, in dry form in a container (e.g., a glass vial), and optionally a vial with a solvent.

In some aspects, the kit or product of manufacture further comprises at least one container with a cationic carrier unit or a micelle disclosed herein (e.g., a glass vial) and a second container with the micelle's payload (e.g., a SETD7 modulator of the present disclosure).

In some aspects, the kit or product of manufacture comprises a cationic carrier unit disclosed herein in a dry form and the micelle's payload (e.g., a SETD7 modulator of the present disclosure) also in dry form in the same container, or in different containers. In some aspects, the kit or product of manufacture comprises a cationic carrier unit disclosed herein in solution and the micelle's payload (e.g., a SETD7 modulator of the present disclosure) also in solution in the same container, or in different containers.

In some aspects, the kit or product of manufacture comprises a micelle disclosed herein in solution, wherein a SETD7 modulator of the present disclosure is encapsulated in the micelle, and instructions for use. In some aspects, the kit or product of manufacture comprises a micelle of disclosed herein in dry form, wherein a SETD7 modulator of the present disclosure is encapsulated in the micelle, and instructions for use (e.g., instructions for reconstitution and administration).

SEQUENCES

| SEQ | | |
|---|---|---|
| ID | | |
| NO | Description | Sequence |
| 1 | Pattern/Motif X₁, X₂, and X₃ is Serine | X₁VAYGYDHX₂PPGKX₃ |
| | (S) or Threonine (T); a phosphomimetic | |
| | amino acid or analog thereof; or, a | |
| | combination thereof | |
| 2 | Wild type | TVAYGYDHSPPGKS |
| 3 | Derivative from native; including C- and | x-TVAYGYDHSPPGKS-y |
| | N- terminal synthetic modifications, x is | |
| | not NH2, y is not COOH | |
| 4 | Phosphorylated peptide, T substituted | SVAYGYDHSPPGKS |
| 5 | Phosphorylated peptide, 1^{st} S substituted | TVAYGYDHTPPGKS |
| 6 | Phosphorylated peptide, 2^{nd} S substituted | TVAYGYDHSPPGKT |
| 7 | Phosphorylated peptide, T and 1^{st} S | SVAYGYDHTPPGKS |
| | substituted | |
| 8 | Phosphorylated peptide, T and 2^{nd} S | SVAYGYDHSPPGKT |
| | substituted | |
| 9 | Phosphorylated peptide, 1^{st} S and 2^{nd} S | TVAYGYDHTPPGKT |
| | substituted | |
| 10 | Phosphorylated peptide, all substituted | SVAYGYDHTPPGKT |
| 11 | Non-phosphorylated peptide with | EVAYGYDHEPPGKE |
| | phosphomimetic D/E amino acids, all E | |
| 12 | Non-phosphorylated peptide with | DVAYGYDHDPPGKD |
| | phosphomimetic D/E amino acids, all D | |
| 13 | Non-phosphorylated peptide with | EVAYGYDHDPPGKE |
| | phosphomimetic D/E amino acids | |
| 14 | Non-phosphorylated peptide with | EVAYGYDHEPPGKD |
| | phosphomimetic D/E amino acids | |
| 15 | Non-phosphorylated peptide with | EVAYGYDHDPPGKD |
| | phosphomimetic D/E amino acids | |
| 16 | Non-phosphorylated peptide with | DVAYGYDHEPPGKE |
| | phosphomimetic D/E amino acids | |
| 17 | Non-phosphorylated peptide with | DVAYGYDHDPPGKE |
| | phosphomimetic D/E amino acids | |
| 18 | Non-phosphorylated peptide with | DVAYGYDHEPPGKD |
| | phosphomimetic D/E amino acids | |
| 19 | X₄, X₇ and X₈ are serine, threonine, | X₄VAX5GX₆DHX₇PPGKX₈ |
| | aspartic acid, glutamic acid, 2-amino- | |
| | 4-phosphobutyric acid, or | |
| | thiophosphoserine; and X₅ and X₆ are | |
| | tyrosine or 4-phosphomethyl-L- | |
| | phenylalanine. | |
| 20 | N-term extension to SEQ ID NO: 19 | DEEL |
| 21 | N-term extension to SEQ ID NO: 19 | ADEEL |
| 22 | C-term extension to SEQ ID NO: 19 | GPEA |
| 23 | C-term extension to SEQ ID NO: 19 | GPEAP |
| 24 | Extended phosphomimetic peptide EEE | ADEELEVAYGYDHEPPGKEGPEAP |
| 25 | Extended phosphomimetic peptide EDE | ADEELEVAYGYDHDPPGKEGPEAP |
| 26 | Extended phosphomimetic peptide EED | ADEELEVAYGYDHEPPGKDGPEAP |
| 27 | Extended phosphomimetic peptide EDD | ADEELEVAYGYDHDPPGKDGPEAP |
| 28 | Extended phosphomimetic peptide DEE | ADEELDVAYGYDHEPPGKEGPEAP |
| 29 | Extended phosphomimetic peptide DED | ADEELDVAYGYDHEPPGKDGPEAP |
| 30 | Extended phosphomimetic peptide DDE | ADEELDVAYGYDHDPPGKEGPEAP |
| 31 | Extended phosphomimetic peptide DDD | ADEELDVAYGYDHDPPGKDGPEAP |
| 32 | Extended phosphomimetic peptide | ADEELTVAYGYDHSPPGKSGPEAP |
| 33 | Phospho-mimicking mRNA sequence | GCNGAYGARGARYUNGARGUNGCNUAYGGNUAYGA |
| | [N: any nucleotide (A,G,T,C), Y: | YCAYGAYCCNCCNGGNAARGAYGGNCCNGARGCNC |
| | pyrimidine (C,T), R: purine (A,G)] | CNURR |
| 34 | Phospho-mimicking mRNA sequence | GCNGAYGARGARYUNACNGUNGCNUAYGGNUAYGA |
| | [N: any nucleotide (A,G,T,C), Y: | YCAYWSNCCNCCNGGNAARWSNGGNCCNGARGCNC |
| | pyrimidine (C,T), R: purine (A,G), W: | CNURR |
| | Weak (A,T), S: Strong (C,G)] | |
| 35 | Enzymatically inactive/phospho-mimicking | MDSDDEMVEEAVEGHLDDDGLPHGFCTVTYSSTDR |
| | mutant aa sequence (X = phosphomimetic amino | FEGNFVHGEKNGRGKFFFFDGSTLEGYYVDDALQG |
| | acid, e.g., acidic amino acid such as D or E) | QGVYTYEDGGVLQGTYVDGELNGPAQEYDTDGRLI |
| | Substrate binding sites (Tyr245, Lys317, | FKGQYKDNIRHGVCWIYYPDGGSLVGEVNEDGEMT |
| | Tyr335) and S-adenosyl-L-methionine | GEKIAYVYPDERTALYGKFIDGEMIEGKLATLMST |
| | binding site (Glu356) are highlighted. | EEGRPHFELMPGNSVYHFDKSTSSCISTNALLPDP |
| | H297A mutant | 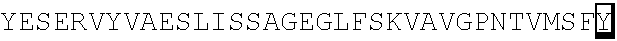 |
| | | NGVRITHQEVDSRDWALNGNTLSLDEETVIDVPEP |
| | | 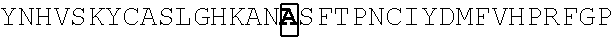 |
| | | 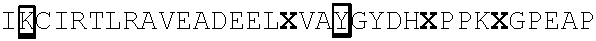 |
| | | 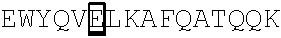 |
| 36 | Enzymatically inactive/phospho-mimicking | AUGGAYWSNGAYGAYGARAUGGUNGARGARGCNGU |
| | mutant mRNA sequence | NGARGGNCAYYUNGAYGAYGAYGGNYUNCCNCAYG |
| | [N: any nucleotide (A,G,T,C), Y: | GNUUYUGYACNGUNACNUAYWSNWSNACNGAYMGN |
| | pyrimidine (C,T), R: purine (A,G), W: | UUYGARGGNAAYUUYGUNCAYGGNGARAARAAYGG |
| | Weak (A,T), S: Strong (C,G)] | NMGNGGNAARUUYUUYUUYUUYGAYGGNWSNACNY |
| | | UNGARGGNUAYUAYGUNGAYGAYGCNYUNCARGGN |
| | | CARGGNGUNUAYACNUAYGARGAYGGNGGNGUNYU |
| | | NCARGGNACNUAYGUNGAYGGNGARYUNAAYGGNC |
| | | CNGCNCARGARUAYGAYACNGAYGGNMGNYUNAUH |
| | | UUYAARGGNCARUAYAARGAYAAYAUHMGNCAYGG |
| | | NGUNUGYUGGAUHUAYUAYCCNGAYGGNGGNWSNY |
| | | UNGUNGGNGARGUNAAYGARGAYGGNGARAUGACN |
| | | GGNGARAARAUHGCNUAYGUNUAYCCNGAYGARMG |
| | | NACNGCNYUNUAYGGNAARUUYAUHGAYGGNGARA |
| | | UGAUHGARGGNAARYUNGCNACNYUNAUGWSNACN |
| | | GARGARGGNMGNCCNCAYUUYGARYUNAUGCCNGG |
| | | NAAYWSNGUNUAYCAYUUYGAYAARWSNACNWSNW |
| | | SNUGYAUHWSNACNAAYGCNYUNYUNCCNGAYCCN |
| | | UAYGARWSNGARMGNGUNUAYGUNGCNGARWSNYU |
| | | NAUHWSNWSNGCNGGNGARGGNYUNUUYWSNAARG |
| | | UNGCNGUNGGNCCNAAYACNGUNAUGWSNUUYUAY |
| | | AAYGGNGUNMGNAUHACNCAYCARGARGUNGAYWS |
| | | NMGNGAYUGGGCNYUNAAYGGNAAYACNYUNWSNY |
| | | UNGAYGARGARACNGUNAUHGAYGUNCCNGARCCN |
| | | UAYAAYCAYGUNWSNAARUAYUGYGCNWSNYUNGG |
| | | NCAYAARGCNAAYGCNWSNUUYACNCCNAAYUGYA |
| | | UHUAYGAYAUGUUYGUNCAYCCNMGNUUYGGNCCN |
| | | AUHAARUGYAUHMGNACNYUNMGNGCNGUNGARGC |
| | | NGAYGARGARYUNGARGUNGCNUAYGGNUAYGAYC |
| | | AYGAYCCNCCNGGNAARGAYGGNCCNGARGCNCCN |
| | | GARUGGUAYCARGUNGARYUNAARGCNUUYCARGO |
| | | NACNCARCARAAR |
| 37 | SEDT7 WT full length amino acid | MDSDDEMVEEAVEGHLDDDGLPHGFCTVTYSSTDR |
| | sequence. Core peptide between arrows. | FEGNFVHGEKNGRGKFFFFDGSTLEGYYVDDALQG |
| | Extended peptide boxed. SET domain | QGVYTYEDGGVLQGTYVDGELNGPAQEYDTDGRLI |
| | is underlined | FKGQYKDNIRHGVCWIYYPDGGSLVGEVNEDGEMT |
| | | GEKIAYVYPDERTALYGKFIDGEMIEGKLATLMST |
| | | EEGRPHFELMPGNSVYHFDKSTSSCISTNALLPDP |
| | | YESERVYVAESLISSAGEGLFSKVAVGPNTVMSFY |
| | | NGVRITHQEVDSRDWALNGNTLSLDEETVIDVPEP |
| | | YNHVSKYCASLGHKANHSFTPNCIYDMFVHPRFGP |
| | | 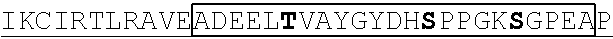 |
| | | EWYQVELKAFQATQQK |
| 38 | Phospho-mimicking peptide sequence | ADEELXVAYGYDHXPPGKXGPEAP |
| | (X = acidic amino acid: D or E) | |
| 39 | Homo sapiens SET domain containing 7, | ATGGATAGCGACGACGAGATGGTGGAGGAGGCGGT |
| | histone lysine methyltransferase | GGAAGGGCACCTGGACGATGACGGATTACCGCACG |
| | (SETD7), transcript variant 1, mRNA | GGTTCTGCACAGTCACCTACTCCTCCACAGACAGA |
| | NCBI Reference Sequence: | TTTGAGGGGAACTTTGTTCACGGAGAAAAGAACGG |
| | NM_030648.3 | ACGGGGGAAGTTCTTCTTCTTTGATGGCAGCACCC |
| | | TGGAGGGGTATTATGTGGATGATGCCTTGCAGGGC |
| | | CAGGGAGTTTACACTTACGAAGATGGGGGAGTTCT |
| | | CCAGGGCACGTATGTAGACGGAGAGCTGAACGGTC |
| | | CAGCCCAGGAATATGACACAGATGGGAGACTGATC |
| | | TTCAAGGGGCAGTATAAAGATAACATTCGTCATGG |
| | | AGTGTGCTGGATATATTACCCAGATGGAGGAAGCC |
| | | TTGTAGGAGAAGTAAATGAAGATGGGGAGATGACT |
| | | GGAGAGAAGATAGCCTATGTGTACCCTGATGAGAG |
| | | GACCGCACTTTATGGGAAATTTATTGATGGAGAGA |
| | | TGATAGAAGGCAAACTGGCTACCCTTATGTCCACT |
| | | GAAGAAGGGAGGCCTCACTTTGAACTGATGCCTGG |
| | | AAATTCAGTGTACCACTTTGATAAGTCGACTTCAT |
| | | CTTGCATTTCTACCAATGCTCTTCTTCCAGATCCT |
| | | TATGAATCAGAAAGGGTTTATGTTGCTGAATCTCT |
| | | TATTTCCAGTGCTGGAGAAGGACTTTTTTCAAAGG |
| | | TAGCTGTGGGACCTAATACTGTTATGTCTTTTTAT |
| | | AATGGAGTTCGAATTACACACCAAGAGGTTGACAG |
| | | CAGGGACTGGGCCCTTAATGGGAACACCCTCTCCC |
| | | TTGATGAAGAAACGGTCATTGATGTGCCTGAGCCC |
| | | TATAACCACGTATCCAAGTACTGTGCCTCCTTGGG |
| | | ACACAAGGCAAATCACTCCTTCACTCCAAACTGCA |
| | | TCTACGATATGTTTGTCCACCCCCGTTTTGGGCCC |
| | | ATCAAATGCATCCGCACCCTGAGAGCAGTGGAGGC |
| | | CGATGAAGAGCTCACCGTTGCCTATGGCTATGACC |
| | | ACAGCCCCCCCGGGAAGAGTGGGCCTGAAGCCCCT |
| | | GAGTGGTACCAGGTGGAGCTGAAGGCCTTCCAGGC |
| | | CACCCAGCAAAAGTGA |

The following examples are offered by way of illustration and not by way of limitation.

### EXAMPLES

### Example 1

### SETD7 Protein Expression in Different Tissues and Identification of Phosphorylation Sites

Tissue samples obtained from normal mice were taken to determine SETD7 protein expression levels. Proteins were extracted from the tissues and protein expression levels were measured using Western blot. SETD7 was observed to be specifically expressed in the brain and eye (results not shown).

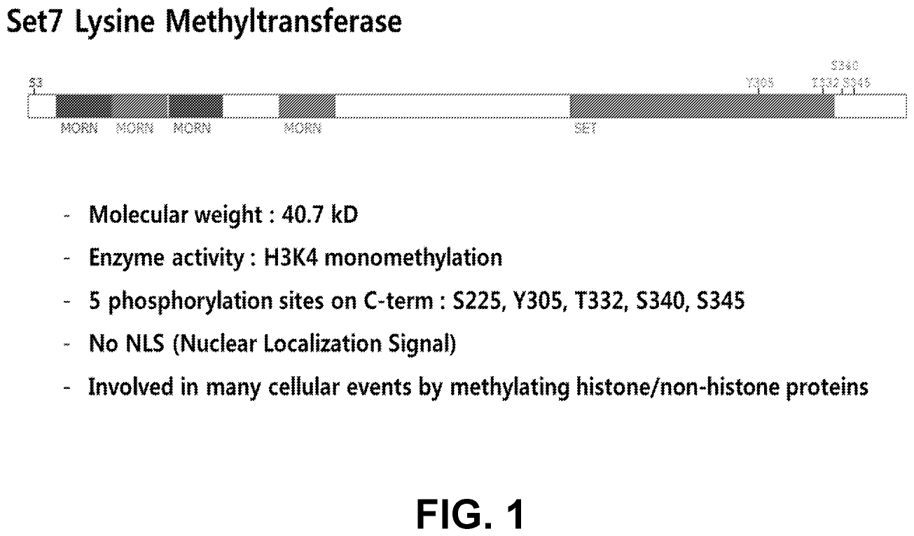

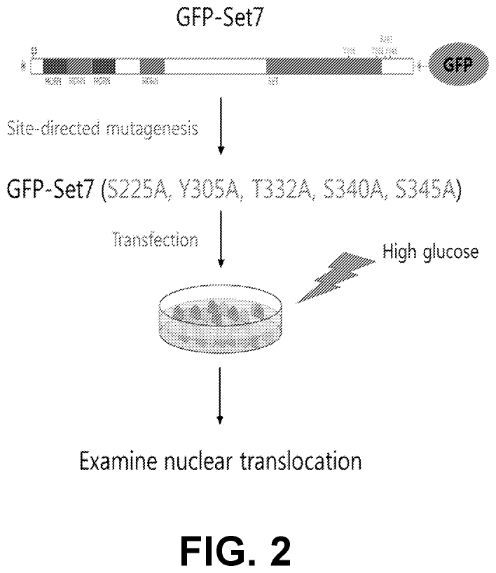

To identify the potential role of phosphorylatable sites located in the C-terminal regions of SETD7 (FIG. 1), phosphorylatable serines, threonines and tyrosines at positions 225, 305, 332, 340, and 345 where mutated to alanine (phospho-disrupting mutations) and expressed in human retinal pigment epithelial cells (RPE). Nuclear translocation or lack thereof in response to high glucose levels was examined (FIG. 2).

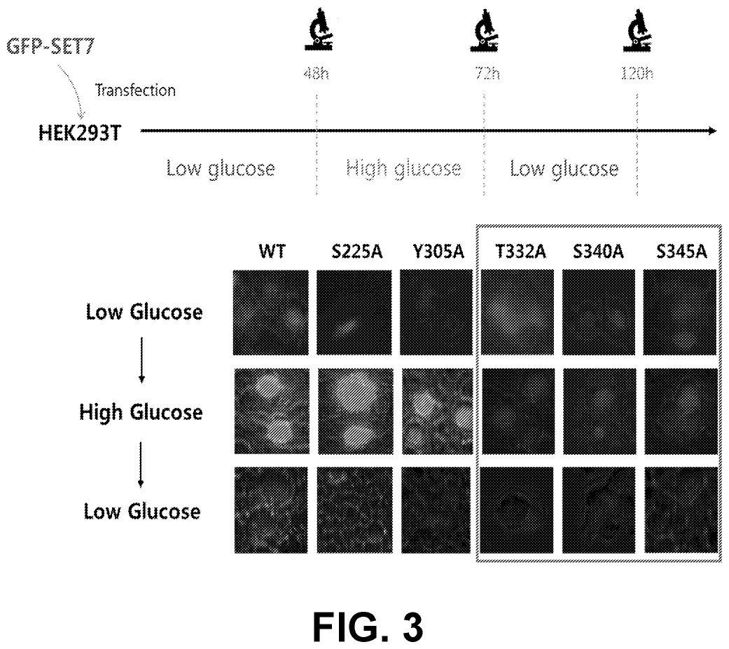

SETD7 cellular distribution changed in response to changes in glucose levels for wild type SETD7 (WT) and SETD7 mutants comprising a S225A substitution or a Y305A substitutions. WT SETD7 as well as the S225A and Y305A SETD7 mutants translocated normally to the nucleus. SETD7 mutants with T332A, S340A or S345 substitutions did not translocate to the nucleus in response to changes in glucose, indicating that phosphorylation at any of those positions is required for glucose-mediated nuclear translocation (FIG. 3).

### Example 2

### Expression of SETD7 Mutants in Cells Under Low or High Glucose Conditions

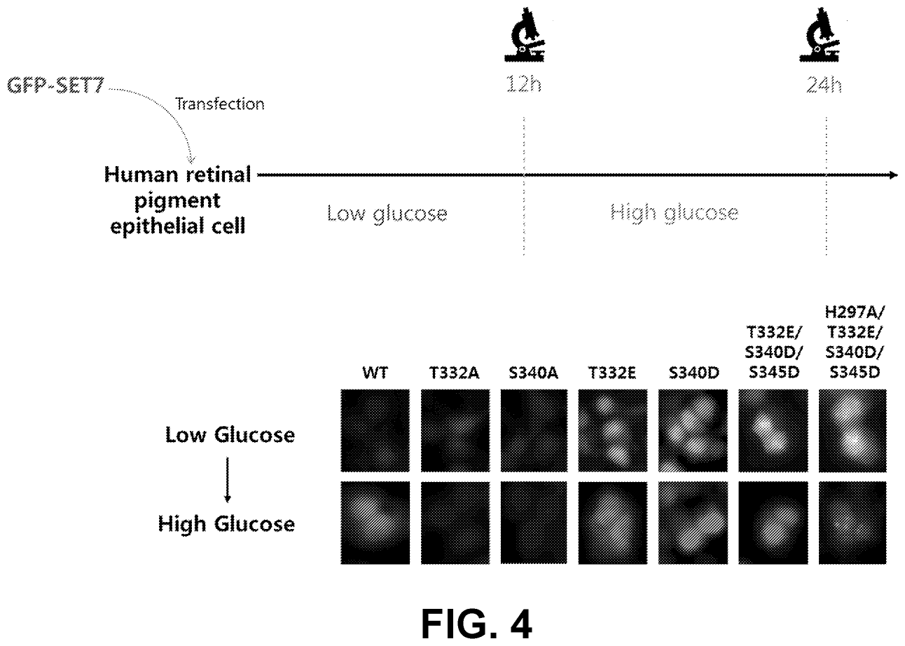

SETD7-GFP mutants with phospho-mimicking substitutions at positions 332, 340 and/or 345 were prepared. The phospho-mimicking substitutions were T332E, S340D, and/or S345D. The SETD7-GFP mutants were expressed in human retinal pigment epithelial (RPE) cells. The fluorescence intensity emitted from the GFP signal in the cells under high or low glucose condition was measured. SETD7 T332A and SETD7 S340A mutants, which could not be phosphorylated, were not translocated to the nucleus in the presence of low or high glucose levels. SETD7 with phospho-mimetic substitutions, such as SETD7 T332E, SETD7 S340D, and SETD7 T332E/S340D/S345D were translocated to the nucleus under low glucose and high glucose conditions. SETD7 H297A/T332E/S340D/S345D (catalytically inert mutant due to the H297A mutation) also translocated to the nucleus under low glucose and high glucoses conditions, i.e., the catalytically inert SETD7 protein accumulated in the nucleus regardless of glucose concentration (FIG. 4).

### Example 3

### Increase of Inflammatory Gene Expression by Overexpression of SETD7 Protein

Human retinal pigment epithelial cells will be transfected with wild type SETD7, phospho-mimicking constructs (e.g., comprising D or E substitutions at positions 332, 340, 345, or combinations thereof), or catalytically inactive mutants (e.g., H297A). The cells will be cultured under high glucose conditions (20 mM glucose) and mRNA and/or protein expression levels of inflammatory genes (e.g., NF-kb, IL-1β, etc) will be measured. The wild type and phospho-mimicking SETD7 constructs are expected to show increased inflammatory gene and protein expression while the catalytically inactive SETD7 mutant is expected to show reduced inflammatory gene and protein expression under high glucose conditions.

### Example 4

### Control of Inflammatory Gene Promoter by SETD7 Protein

In order to test whether SETD7 protein controls transcription of the inflammatory genes in human retinal pigment epithelial (RPE) cells, the cells will be transfected with wild type SETD7 constructs, phospho-disrupting mutants (e.g., comprising A substitutions at positions 332, 340, 345, or combinations thereof), and phospho-mimicking mutants (e.g., comprising D or E substitutions at positions 332, 340, 345, or combinations thereof). The cells will be cultured under low glucose or high glucose conditions and will be assayed with a chromatin immunoprecipitation assay.

Wild type SETD7 protein is expected not to interact with the promoters of inflammatory genes, but is expected to be recruited to the inflammatory gene promoters when exposed to high glucose. SETD7 phospho-disrupting mutants are expected not to be recruited to the inflammatory gene promoter regardless of the glucose concentration. The SETD7 phospho-mimicking mutants are expected to interact with the inflammatory gene promoters regardless of the glucose concentration.

### Example 5

### Competitive Inhibitor Suppressing Nuclear Translocation of SETD7 Protein

The RPE cells will be transfected with cDNA encoding phospho-mimicking mutants (i.e., T332, 5340, and/or 5345 substituted with glutamate or aspartate). The cells will be cultured under low glucose or high glucose conditions and will be assayed for the cellular expression and inflammatory gene expression. Cells transfected with the phospho-mimicking mutants are expected to show reduced SETD7 nuclear translocation compared to the control and reduced inflammatory gene expression.

### Example 6

### Effects of SETD7 Competitive Inhibitor on Diabetic Retinopathy

In order to study the effect of a competitive inhibitor of SETD7 nuclear translocation in diabetic retinopathy, mouse models will be induced to have diabetes using streptozotocin (STZ). mRNAs encoding various SETD7 mutants will be administered to the mouse eyes via intravitreal injection.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

The contents of all cited references (including literature references, patents, patent applications, and websites) that may be cited throughout this application are hereby expressly incorporated by reference in their entirety for any purpose, as are the references cited therein.

What is claimed is:

1. An isolated polynucleotide encoding a polypeptide which comprises the sequence
(SEQ ID NO: 1)
X₁VAYGYDHX₂PPGKX₃,
wherein at least one of X₁, X₂, and X₃ is
(i) Serine (S) or Threonine (T);
(ii) a phospho-mimetic amino acid or analog thereof, or,
(iii) a combination thereof,
wherein the polypeptide sequence is not TVAYGYDHSPPGKS (SEQ ID NO: 2; wild type),
wherein one, two, three, four or five amino acids other than X₁, X₂, and X₃ are optionally substituted with respect to their corresponding amino acids in SEQ ID NO: 2, and
wherein the polypeptide is capable of modulating nuclear translocation of endogenous SET domain containing 7, histone lysine methyltransferase (SETD7).

2. The isolated polynucleotide of claim 1, wherein the phospho-mimetic amino acid is Aspartic acid (D) or Glutamic acid (E).

3. The isolated polynucleotide of claim 1, wherein the phospho-mimetic amino acid is phosphoserine or phosphothreonine.

4. The isolated polynucleotide of claim 1, wherein the phospho-mimetic amino acid analog is a non-cleavable analog.

5. The isolated polynucleotide of claim 4, wherein the non-cleavable analog is a phosphoserine non-hydrolyzable analog.

6. The isolated polynucleotide of claim 5, wherein the non-hydrolyzable analog of phosphoserine is 2-amino-4-phosphobutyric acid.

7. The isolated polynucleotide of any one of claims 1 to 6, wherein the optionally substituted amino acids are conservative amino acid substitutions.

8. The isolated polynucleotide of claim 1, where at least one tyrosine (Y) is substituted.

9. The isolated polynucleotide of claim 8, wherein at least one tyrosine is substituted with phosphotyrosine, aspartic acid, glutamic acid, or an analog thereof.

10. The isolated polynucleotide of claim 9, wherein the phosphotyrosine analog is a non-hydrolyzable analog.

11. The isolated polynucleotide of claim 10, wherein the non-hydrolyzable analog of phosphotyrosine is 4-phosphomethyl-L-phenylalanine (Pmp).

12. The isolated polynucleotide of claim 1, wherein the phosphomimetic amino acid analog is a thiophosphate analog.

13. The isolated polynucleotide of claim 12, wherein the thiophosphate analog is thiophosphoserine.

14. The isolated polynucleotide of claim 1, wherein the sequence of the polypeptide comprises a sequence selected from the group consisting of

| | | |
|---|---|---|
| a. | x-TVAYGYDHSPPGKS-y; | (SEQ ID NO: 3) |
| b. | SVAYGYDHSPPGKS; | (SEQ ID NO: 4) |
| c. | TVAYGYDHTPPGKS; | (SEQ ID NO: 5) |
| d. | TVAYGYDHSPPGKT; | (SEQ ID NO: 6) |
| e. | SVAYGYDHTPPGKS; | (SEQ ID NO: 7) |
| f. | SVAYGYDHSPPGKT; | (SEQ ID NO: 8) |
| g. | TVAYGYDHTPPGKT; | (SEQ ID NO: 9) |
| h. | SVAYGYDHTPPGKT; | (SEQ ID NO: 10) |
| i. | EVAYGYDHEPPGKE; | (SEQ ID NO: 11) |
| j. | DVAYGYDHDPPGKD; | (SEQ ID NO: 12) |
| k. | EVAYGYDHDPPGKE; | (SEQ ID NO: 13) |
| l. | EVAYGYDHEPPGKD; | (SEQ ID NO: 14) |
| m. | EVAYGYDHDPPGKD; | (SEQ ID NO: 15) |
| n. | DVAYGYDHEPPGKE; | (SEQ ID NO: 16) |
| o. | DVAYGYDHDPPGKE; | (SEQ ID NO: 17) |
| and, | | |
| p. | DVAYGYDHEPPGKD; | (SEQ ID NO: 18) |

wherein x is an N-terminal modification and y is a C-terminal modification.

15. The isolated polynucleotide of claim 14, wherein the N-terminal modification is acetylation.

16. The isolated polynucleotide of claim 14, wherein the C-terminal modification is amidation.

17. The isolated polynucleotide of claim 8, wherein the polypeptide which comprises the sequence
(SEQ ID NO: 19)
X₄VAX₅GX₆DHX₇PPGKX₈
wherein X₄, X₇ and X₈ are selected from the group consisting of serine, threonine, aspartic acid, glutamic acid, 2-amino-4-phosphobutyric acid, and thiophosphoserine; and
wherein X₅ and X₆ are selected from the group consisting of tyrosine, and 4-phosphomethyl-L-phenylalanine.

18. The isolated polynucleotide of any one of claims 1 to 17, wherein the polypeptide further comprises
(i) an L, EL, EEL, DEEL (SEQ ID NO:20), or ADEEL (SEQ ID:21) amino acid sequence appended to its N-terminus;
(ii) a G, GP, GPE, GPEA (SEQ ID NO:22), or GPEAP (SEQ ID NO:23) amino acid sequence appended to its C-terminus; or,
(iii) a combination thereof.

19. The isolated polynucleotide of any one of claims 1 to 18, wherein the polypeptide comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence as set forth in:

| | | |
|---|---|---|
| (i) | ADEELEVAYGYDHEPPGKEGPEAP; | (SEQ ID NO: 24) |
| (ii) | ADEELEVAYGYDHDPPGKEGPEAP; | (SEQ ID NO: 25) |
| (iii) | ADEELEVAYGYDHEPPGKDGPEAP; | (SEQ ID NO: 26) |
| (iv) | ADEELEVAYGYDHDPPGKDGPEAP; | (SEQ ID NO: 27) |
| (v) | ADEELDVAYGYDHEPPGKEGPEAP; | (SEQ ID NO: 28) |
| (vi) | ADEELDVAYGYDHEPPGKDGPEAP; | (SEQ ID NO: 29) |
| (vii) | ADEELDVAYGYDHDPPGKEGPEAP; | (SEQ ID NO: 30) |
| or | | |
| (viii) | ADEELDVAYGYDHDPPGKDGPEAP. | (SEQ ID NO: 31) |

20. The isolated polynucleotide of any one of claims 1 to 18, wherein the polypeptide consists of or consists essentially of the sequence as set forth in:

| | | |
|---|---|---|
| (i) | ADEELEVAYGYDHEPPGKEGPEAP; | (SEQ ID NO: 24) |
| (ii) | ADEELEVAYGYDHDPPGKEGPEAP; | (SEQ ID NO: 25) |
| (iii) | ADEELEVAYGYDHEPPGKDGPEAP; | (SEQ ID NO: 26) |
| (iv) | ADEELEVAYGYDHDPPGKDGPEAP; | (SEQ ID NO: 27) |
| (v) | ADEELDVAYGYDHEPPGKEGPEAP; | (SEQ ID NO: 28) |
| (vi) | ADEELDVAYGYDHEPPGKDGPEAP; | (SEQ ID NO: 29) |
| (vii) | ADEELDVAYGYDHDPPGKEGPEAP; | (SEQ ID NO: 30) |
| or | | |
| (viii) | ADEELDVAYGYDHDPPGKDGPEAP. | (SEQ ID NO: 31) |

21. An isolated polynucleotide encoding a polypeptide which consists of or consists essentially of the sequence as set forth in SEQ ID NO: 2 (TVAYGYDHSPPGKS) and
(i) an L, EL, EEL, DEEL (SEQ ID NO:20), or ADEEL (SEQ ID:21) amino acid sequence appended to its N-terminus;
(ii) a G, GP, GPE, GPEA (SEQ ID NO:22), or GPEAP (SEQ ID NO:23) amino acid sequence appended to its C-terminus; or,
(iii) a combination thereof.

22. The isolated polynucleotide of claim 21, wherein the polypeptide consists essentially of or consists of an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence as set forth in ADEELTVAYGYDHSPPGKSGPEAP (SEQ ID NO:32).

23. The isolated polynucleotide of claim 22 wherein the polypeptide consists or consists essentially of ADEELTVAYGYDHSPPGKSGPEAP (SEQ ID NO:32).

24. The isolated polynucleotide of any one of claims 1 to 23, wherein the polypeptide has at least 14 amino acids, at least 15 amino acids, at least 16 amino acids, at least 17 amino acids, at least 18 amino acids, at least 19 amino acids, at least 20 amino acids, at least 21 amino acids, at least 22 amino acids, at least 23 amino acids, or at least 24 amino acids in length.

25. The isolated polynucleotide of any one of claims 1 to 21, wherein the polypeptide comprises an N-terminal capping modification, a C-terminal capping modification, or a combination thereof.

26. The isolated polynucleotide of claim 25, wherein the N-terminal capping modification is an N-terminal acetylation, formylation, acylation, pyroglutamylation, or carbamate, sulfonamide, or alkylamine modification.

27. The isolated polynucleotide of claim 25, wherein the C-terminal capping modification is a C-terminal amidation, N-alkyl amidation, aldehyde modification, or esterification.

28. The isolated polynucleotide of any one of claims 1 to 27, which comprises a nucleic acid sequence at least about 70%, at least about 75%, at least about 80%, at least about 85%, at least about 90%, at least about 95%, at least about 96%, at least about 97%, at least about 98%, at least about 99%, or about 100% sequence identity to the sequence as set forth in 5′-GCNGAYGARGARYUNGARGUNGCNUAYGGNUAYGAYCAYGAYCCNCCNGGNAAR GAYGGNCCNGARGCNCCNURR-3′ (SEQ ID NO:33), wherein N is any nucleotide (A, G, T, or C), Y is a pyrimidine (C or T), R is a purine (A or G).

29. The isolated polynucleotide of any one of claims 1 to 27, which comprises a nucleic acid sequence as set forth in 5′-GCNGAYGARGARYUNGARGUNGCNUAYGGNUAYGAYCAYGAYCCNCCNGGNAAR GAYGGNCCNGARGCNCCNURR-3′ (SEQ ID NO:33), wherein N is any nucleotide (A, G, T, or C), Y is a pyrimidine (C or T), R is a purine (A or G).

30. The isolated polynucleotide of any one of claims 1 to 27, which consists of or consists essentially of a nucleic acid sequence at least about 70%, at least about 75%, at least about 80%, at least about 85%, at least about 90%, at least about 95%, at least about 96%, at least about 97%, at least about 98%, at least about 99%, or about 100% sequence identity to the sequence as set forth in 5′-GCNGAYGARGARYUNACNGUNGCNUAYGGNUAYGAYCAYWSNCCNCCNGGNAAR WSNGGNCCNGARGCNCCNURR-3′ (SEQ ID NO: 34).

31. The isolated polynucleotide of any one of claims 1 to 30, wherein the polypeptide comprises at least about 20 amino acids, at least about 30 amino acids, at least about 40 amino acids, at least about 50 amino acids, at least about 60 amino acids, at least about 70 amino acids, at least about 80 amino acids, at least about 90 amino acids, at least about 100 amino acids, at least about 120 amino acids, at least about 140 amino acids, at least about 160 amino acids, at least about 180 amino acids, at least about 200 amino acids, at least about 220 amino acids, at least about 240 amino acids, at least about 260 amino acids, at least about 280 amino acids, at least about 300 amino acids, at least about 320 amino acids, at least about 340 amino acids or at least about 360 amino acids in length.

32. The isolated polynucleotide of any one of claims 1 to 31, wherein the polypeptide is enzymatically inactive.

33. The isolated polynucleotide of claim 32, wherein the enzymatically inactive polypeptide comprises an amino acid other than His at amino acid residue 297 corresponding to SEQ ID NO: 35.

34. The isolated polynucleotide of claim 33, wherein the amino acid at residue 297 corresponding to SEQ ID NO: 35 is Ala.

35. An isolated polynucleotide encoding a polypeptide, wherein the polypeptide comprises alanine at amino acid residue 297 corresponding to SEQ ID NO: 35.

36. The isolated polynucleotide of any one of claims 1 to 35, wherein the polypeptide comprises at least about 70%, at least about 75%, at least about 80%, at least about 85%, at least about 90%, at least about 95%, at least about 96%, at least about 97%, at least about 98%, at least about 99%, or about 100% sequence identity to the sequence as set forth in:
(SEQ ID NO: 35)
MDSDDEMVEEAVEGHLDDDGLPHGFCTVTYSSTDRFEGNFVHGEKNGRGK
FFFFDGSTLEGYYVDDALQGQGVYTYEDGGVLQGTYVDGELNGPAQEYDT
DGRLIFKGQYKDNIRHGVCWIYYPDGGSLVGEVNEDGEMTGEKIAYVYPD
ERTALYGKFIDGEMIEGKLATLMSTEEGRPHFELMPGNSVYHFDKSTSSC
ISTNALLPDPYESERVYVAESLISSAGEGLFSKVAVGPNTVMSFYNGVRI
THQEVDSRDWALNGNTLSLDEETVIDVPEPYNHVSKYCASLGHKANASFT
PNCIYDMFVHPRFGPIKCIRTLRAVEADEELXVAYGYDHXPPGKXGPEAP
EWYQVELKAFQATQQK.

37. The isolated polynucleotide of claim 36, wherein the polypeptide consists of or consists essentially of the sequence as set forth in:
(SEQ ID NO: 35)
MDSDDEMVEEAVEGHLDDDGLPHGFCTVTYSSTDRFEGNFVHGEKNGRGK
FFFFDGSTLEGYYVDDALQGQGVYTYEDGGVLQGTYVDGELNGPAQEYDT
DGRLIFKGQYKDNIRHGVCWIYYPDGGSLVGEVNEDGEMTGEKIAYVYPD
ERTALYGKFIDGEMIEGKLATLMSTEEGRPHFELMPGNSVYHFDKSTSSC
ISTNALLPDPYESERVYVAESLISSAGEGLFSKVAVGPNTVMSFYNGVRI
THQEVDSRDWALNGNTLSLDEETVIDVPEPYNHVSKYCASLGHKANASFT
PNCIYDMFVHPRFGPIKCIRTLRAVEADEELXVAYGYDHXPPGKXGPEAP
FEWYQVELKAFQATQQK.

38. The isolated polynucleotide of claim 36 or 37, which comprises or consists of
5′AUGGAYWSNGAYGAYGARAUGGUNGARGARGCNGUNGARGGNCAYYUN
GAYGAYGAYGGNYUNCCNCAYGGNUUYUGYACNGUNACNUAYWSNWSNAC
NGAYMGNUUYGARGGNAAYUUYGUNCAYGGNGARAARAAYGGNMGNGGNA
ARUUYUUYUUYUUYGAYGGNWSNACNYUNGARGGNUAYUAYGUNGAYGAY
GCNYUNCARGGNCARGGNGUNUAYACNUAYGARGAYGGNGGNGUNYUNCA
RGGNACNUAYGUNGAYGGNGARYUNAAYGGNCCNGCNCARGARUAYGAYA
CNGAYGGNMGNYUNAUHUUYAARGGNCARUAYAARGAYAAYAUHMGNCAY
GGNGUNUGYUGGAUHUAYUAYCCNGAYGGNGGNWSNYUNGUNGGNGARGU
NAAYGARGAYGGNGARAUGACNGGNGARAARAUHGCNUAYGUNUAYCCNG
AYGARMGNACNGCNYUNUAYGGNAARUUYAUHGAYGGNGARAUGAUHGAR
GGNAARYUNGCNACNYUNAUGWSNACNGARGARGGNMGNCCNCAYUUYGA
RYUNAUGCCNGGNAAYWSNGUNUAYCAYUUYGAYAARWSNACNWSNWSNU
GYAUHWSNACNAAYGCNYUNYUNCCNGAYCCNUAYGARWSNGARMGNGUN
UAYGUNGCNGARWSNYUNAUHWSNWSNGCNGGNGARGGNYUNUUYWSNAA
RGUNGCNGUNGGNCCNAAYACNGUNAUGWSNUUYUAYAAYGGNGUNMGNA
UHACNCAYCARGARGUNGAYWSNMGNGAYUGGGCNYUNAAYGGNAAYACN
YUNWSNYUNGAYGARGARACNGUNAUHGAYGUNCCNGARCCNUAYAAYCA
YGUNWSNAARUAYUGYGCNWSNYUNGGNCAYAARGCNAAYGCNWSNUUYA
CNCCNAAYUGYAUHUAYGAYAUGUUYGUNCAYCCNMGNUUYGGNCCNAUH
AARUGYAUHMGNACNYUNMGNGCNGUNGARGCNGAYGARGARYUNGARGU
NGCNUAYGGNUAYGAYCAYGAYCCNCCNGGNAARGAYGGNCCNGARGCNC
CNGARUGGUAYCARGUNGARYUNAARGCNUUYCARGCNACNCARCARAAR
3′
(SEQ ID NO: 36), wherein N is any nucleotide (A,G,T,C), Y is a pyrimidine (C,T), and R is a purine (A,G).

39. The isolated polynucleotide of any one of claims 1 to 38, wherein the polypeptide further comprises at least one heterologous moiety.

40. The isolated polynucleotide of claim 39, wherein at least one heterologous moiety comprises a serum half-life extending moiety.

41. The isolated polynucleotide of claim 40, wherein the serum half-life extending moiety comprises an Fc region, albumin, albumin binding polypeptide, a fatty acid, PAS, the β subunit of the C-terminal peptide (CTP) of human chorionic gonadotropin, polyethylene glycol (PEG), hydroxyethyl starch (HES), XTEN, albumin-binding small molecules, or a combination thereof.

42. The isolated polynucleotide of claim 40 or claim 41, wherein the serum half-life of the polypeptide at least about 10%, at least about 20%, at least about 30%, at least about 40%, at least about 50%, at least about 60%, at least about 70%, at least about 80%, at least about 90%, or at least about 100% higher than the plasma half-life of a corresponding polypeptide without serum half-life extending moiety.

43. The isolated polynucleotide of claim 39, wherein the at least one heterologous moiety comprises a detectable moiety.

44. The isolated polynucleotide of any one of claims 1 to 43, wherein the polynucleotide is a DNA or an RNA.

45. The isolated polynucleotide of any one of claims 1 to 44, wherein the polynucleotide comprises at least one chemically modified nucleobase, sugar, backbone, or any combination thereof.

46. The isolated polynucleotide of any one of claims 1 to 45, wherein the polynucleotide is codon optimized.

47. A vector comprising the isolated polynucleotide of any one of claims 1 to 46.

48. The vector claim 47, wherein the vector is viral vector.

49. The vector of claim 48, wherein the viral vector is an adenoviral vector or an adenoassociated viral vector.

50. The vector of claim 49, wherein the adenoviral vector is a third generation adenoviral vector.

51. The vector of claim 48, wherein the viral vector is a retroviral vector.

52. The vector of claim 51, wherein the retroviral vector is a lentiviral vector.

53. The vector of claim 52, wherein the lentiviral vector is a third or fourth generation lentiviral vector.

54. A polypeptide encoded by the polynucleotide of any one of claims 1 to 46 or by the vector of any one of claims 47 to 53.

55. A composition comprising the polynucleotide of any one of claims 1 to 46, the vector of any one of claims 47 to 53, or the polypeptide of claim 54, and a delivery agent.

56. The composition of claim 55, wherein the delivery agent comprises a lipidoid, a liposome, a lipoplex, a lipid nanoparticle, a polymeric compound, a peptide, a protein, a cell, a nanoparticle mimic, a nanotube, or a conjugate.

57. The composition of claim 55, wherein the delivery agent comprises a cationic carrier unit comprising
[WP]-L1-[CC]-L2-[AM] (formula I)
or
[WP]-L1-[AM]-L2-[CC] (formula II)
wherein
WP is a water-soluble biopolymer moiety;
CC is a positively charged carrier moiety;
AM is an adjuvant moiety; and,
L1 and L2 are independently optional linkers, and
wherein when mixed with a nucleic acid at an ionic ratio of about 1:1, the cationic carrier unit forms a micelle.

58. The composition of claim 57, wherein the polynucleotide of any one of claims 1 to 46, the vector of any one of claims 47 to 53, or the polypeptide of claim 54 interact with the cationic carrier unit via an ionic bond.

59. The composition of claim 57 or 58, wherein the water-soluble polymer comprises poly(alkylene glycols), poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyglycerol, polyphosphazene, polyoxazolines (“POZ”) poly(N-acryloylmorpholine), or any combinations thereof.

60. The composition of claims 57 to 59, wherein the water-soluble polymer comprises polyethylene glycol (“PEG”), polyglycerol, or poly(propylene glycol) (“PPG”).

61. The composition of any one of claims 57 to 69, wherein the water-soluble polymer comprises: 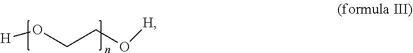
wherein n is 1-1000.

62. The composition of claim 61, wherein the n is at least about 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, or at least about 141.

63. The composition of claim 61, wherein the n is about 80 to about 90, about 90 to about 100, about 100 to about 110, about 110 to about 120, about 120 to about 130, about 140 to about 150, about 150 to about 160.

64. The composition of any one of claims 57 to 63, wherein the water-soluble polymer is linear, branched, or dendritic.

65. The composition of any one of claims 57 to 64, wherein the cationic carrier moiety comprises one or more basic amino acids.

66. The composition of claim 65, wherein the cationic carrier moiety comprises at least three, at least four, at least five, at least six, at least seven, at least eight, at least nine, at least ten, at least 11, at least 12, at least 13, at least 14, at last 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 basic amino acids.

67. The composition of claim 66, wherein the cationic carrier moiety comprises about 30 to about 50 basic amino acids.

68. The composition of claim 66 or claim 67, wherein the basic amino acid comprises arginine, lysine, histidine, or any combination thereof.

69. The composition of any one of claims 57 to 68, wherein the cationic carrier moiety comprises about 40 lysine monomers.

70. The composition of any one of claims 57 to 69, wherein the adjuvant moiety is capable of modulating an immune response, an inflammatory response, and/or a tissue microenvironment.

71. The composition of any one of claims 57 to 70, wherein the adjuvant moiety comprises an imidazole derivative, an amino acid, a vitamin, or any combination thereof.

72. The composition of claim 71, wherein the adjuvant moiety comprises: 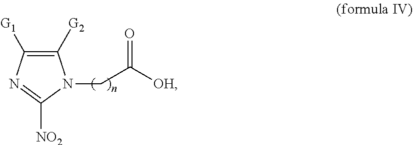
wherein each of G1 and G2 is H, an aromatic ring, or 1-10 alkyl, or G1 and G2 together form an aromatic ring, and wherein n is 1-10.

73. The composition of claim 71, wherein the adjuvant moiety comprises nitroimidazole.

74. The composition of claim 71, wherein the adjuvant moiety comprises metronidazole, tinidazole, nimorazole, dimetridazole, pretomanid, ornidazole, megazol, azanidazole, benznidazole, or any combination thereof.

75. The composition of any one of claims 57 to 70, wherein the adjuvant moiety comprises an amino acid.

76. The composition of claim 75, wherein the adjuvant moiety comprises 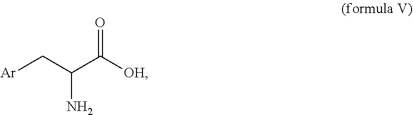
wherein Ar is 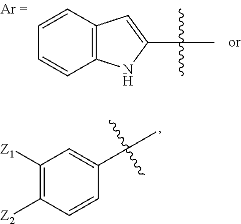
and
wherein each of Z1 and Z2 is H or OH.

77. The composition of any one of claims 57 to 70, wherein the adjuvant moiety comprises a vitamin.

78. The cationic carrier unit of claim 77, wherein the vitamin comprises a cyclic ring or cyclic hetero atom ring and a carboxyl group or hydroxyl group.

79. The composition of claim 77 or claim 78, wherein the vitamin comprises: 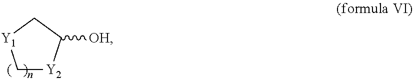
wherein each of Y1 and Y2 is C, N, O, or S, and wherein n is 1 or 2.

80. The composition of any one of claims 77 to 79, wherein the vitamin is selected from the group consisting of vitamin A, vitamin B1, vitamin B2, vitamin B3, vitamin B6, vitamin B7, vitamin B9, vitamin B12, vitamin C, vitamin D2, vitamin D3, vitamin E, vitamin M, vitamin H, and any combination thereof.

81. The composition of any one of claims 77 to 80, wherein the vitamin is vitamin B3.

82. The composition of any one of claims 77 to 81, wherein the adjuvant moiety comprises at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, or at least about 20 vitamin B3.

83. The composition of claim 82, wherein the adjuvant moiety comprises about 10 vitamin B3.

84. The composition of any one of claims 57 to 83, which comprises about a water-soluble biopolymer moiety with about 120 to about 130 PEG units, a cationic carrier moiety comprising a poly-lysine with about 30 to about 40 lysines, and an adjuvant moiety with about 5 to about 10 vitamin B3.

85. A micelle comprising the composition of any one of claims 57 to 84 wherein the polynucleotide of any one of claims 1 to 46, the vector of any one of claims 47 to 53, or the polypeptide of claim 54, and the delivery agent are associated with each other.

86. The micelle of claim 85, wherein the association is a covalent bond, a non-covalent bond, or an ionic bond.

87. The micelle of claim 85 or claim 86, wherein the cationic carrier unit of any one of claims 1 to 31, wherein the positive charge of the cationic carrier moiety of the cationic carrier unit is sufficient to form a micelle when mixed with the polynucleotide of any one of claims 1 to 46, the vector of any one of claims 47 to 53, or the polypeptide of claim 54 in a solution, wherein the overall ionic ratio of the positive charges of the cationic carrier moiety of the cationic carrier unit and the negative charges of the polynucleotide, vector, or polypeptide in the solution is about 1:1.

88. The micelle of any one of claims 85 to 87, wherein the cationic carrier unit is capable of protecting the polynucleotide, vector, or polypeptide from enzymatic degradation.

89. A cell comprising the polynucleotide of any one of claims 1 to 46, or the vector of any one of claims 47 to 53.

90. A pharmaceutical composition comprising the polynucleotide of any one of claims 1 to 46, the vector of any one of claims 47 to 53, the composition of any one of claim 54 to 84, the micelle of any one of claims 85 to 88, or the cell of claim 89, and an excipient.

91. A method for preventing or reducing nuclear translocation of SETD7 in a cell comprising contacting the cell with an effective amount of the polynucleotide of any one of claims 1 to 46, the vector of any one of claims 47 to 53, the composition of any one of claim 54 to 84, the micelle of any one of claims 85 to 88, or the pharmaceutical composition of claim 87.

92. A method for preventing or reducing nuclear accumulation of SETD7 in a cell comprising contacting the cell with an effective amount of the polynucleotide of any one of claims 1 to 46, the vector of any one of claims 47 to 53, the composition of any one of claim 54 to 84, the micelle of any one of claims 85 to 88, or the pharmaceutical composition of claim 90.

93. A method for preventing or reducing histone H3K4 monomethylation in a cell comprising contacting the cell with an effective amount of the polynucleotide of any one of claims 1 to 46, the vector of any one of claims 47 to 53, the composition of any one of claim 54 to 84, the micelle of any one of claims 85 to 88, or the pharmaceutical composition of claim 90.

94. A method for preventing or reducing p53 monomethylation in a cell comprising contacting the cell with an effective amount of the polynucleotide of any one of claims 1 to 46, the vector of any one of claims 47 to 53, the composition of any one of claim 54 to 84, the micelle of any one of claims 85 to 88, or the pharmaceutical composition of claim 90.

95. A method to treat a metabolic disease or disorder in a subject in need thereof comprising administering to the subject a therapeutically effective amount of the polynucleotide of any one of claims 1 to 46, the vector of any one of claims 47 to 53, the composition of any one of claim 54 to 84, the micelle of any one of claims 85 to 88, the cell of claim 89, or the pharmaceutical composition of claim 90.

96. The method of claim 95, wherein the metabolic disease or disorder is diabetes, obesity, or insulin resistance.

97. The method of claim 96, wherein the diabetes is type 2 diabetes mellitus.

98. A method to treat a cancer in a subject in need thereof comprising administering to the subject a therapeutically effective amount of the polynucleotide of any one of claims 1 to 46, the vector of any one of claims 47 to 53, the composition of any one of claim 54 to 84, the micelle of any one of claims 85 to 88, the cell of claim 89, or the pharmaceutical composition of claim 90.

99. A kit comprising the polynucleotide of any one of claims 1 to 46, the vector of any one of claims 47 to 53, the composition of any one of claim 54 to 84, the micelle of any one of claims 85 to 88, the cell of claim 89, or the pharmaceutical composition of claim 90.

100. An article of manufacture comprising the polynucleotide of any one of claims 1 to 46, the vector of any one of claims 47 to 53, the composition of any one of claim 54 to 84, the micelle of any one of claims 85 to 88, the cell of claim 89, or the pharmaceutical composition of claim 90.

101. The kit of claim 99 or article of manufacture of claim 100, further comprising instructions for use according to the methods of claims 91 to 98.

102. A polynucleotide of any one of claims 1 to 46, vector of any one of claims 47 to 53, composition of any one of claim 54 to 84, micelle of any one of claims 85 to 88, cell of claim 89, or pharmaceutical composition of claim 90 for use as a medicament.

103. A polynucleotide of any one of claims 1 to 46, vector of any one of claims 47 to 53, composition of any one of claim 54 to 84, micelle of any one of claims 85 to 88, cell of claim 89, or pharmaceutical composition of claim 90 for use in the treatment of a metabolic disease in a subject in need thereof.

104. The polynucleotide, vector, composition, micelle, cell, or pharmaceutical composition for use according to claim 103, wherein the metabolic disease is diabetes.

105. Use of a polynucleotide of any one of claims 1 to 46, vector of any one of claims 47 to 53, composition of any one of claim 54 to 84, micelle of any one of claims 85 to 88, cell of claim 89, or pharmaceutical composition of claim 90 in the manufacture of treatment for a metabolic disease.

106. The use according to claim 105, wherein the metabolic disease in diabetes.


SEQUENCE LISTING (39 sequences)

SEQ ID NO: 1 - protein, 14 aa, Artificial Sequence
  Synthetic Sequence
  MISC_FEATURE (1)..(1): Xaa is Serine (S), Threonine (T), a phosphomimetic amino acid such as aspartic acid (D) or glutamic acid (E), or an analog thereof
  MISC_FEATURE (9)..(9): Xaa is Serine (S), Threonine (T), a phosphomimetic amino acid such as aspartic acid (D) or glutamic acid (E), or an analog thereof
  MISC_FEATURE (14)..(14): Xaa is Serine (S), Threonine (T), a phosphomimetic amino acid such as aspartic acid (D) or glutamic acid (E), or an analog thereof
XVAYGYDHXPPGKX

SEQ ID NO: 2 - protein, 14 aa, Artificial Sequence
  Wild Type
TVAYGYDHSPPGKS

SEQ ID NO: 3 - protein, 14 aa, Artificial Sequence
  comprises N- and/or C-terminal modification
TVAYGYDHSPPGKS

SEQ ID NO: 4 - protein, 14 aa, Artificial Sequence
  Phosphorylated peptide, T>S substituted
TVAYGYDHSPPGKT

SEQ ID NO: 5 - protein, 14 aa, Artificial Sequence
  Phosphorylated peptide, 1st S of WT substituted
SVAYGYDHTPPGKS

SEQ ID NO: 6 - protein, 14 aa, Artificial Sequence
  Phosphorylated peptide, 2nd S of WT substituted
TVAYGYDHSPPGKT

SEQ ID NO: 7 - protein, 14 aa, Artificial Sequence
  Phosphorylated peptide, T and 1st S of WT substituted
SVAYGYDHTPPGKS

SEQ ID NO: 8 - protein, 14 aa, Artificial Sequence
  Phosphorylated peptide, T and 2nd S of WT substituted
SVAYGYDHSPPGKT

SEQ ID NO: 9 - protein, 14 aa, Artificial Sequence
  Phosphorylated peptide, 1st S and 2nd S of WT substituted
TVAYGYDHTPPGKT

SEQ ID NO: 10 - protein, 14 aa, Artificial Sequence
  Phosphorylated peptide, all S and T of of WT substituted
SVAYGYDHTPPGKT

SEQ ID NO: 11 - protein, 14 aa, Artificial Sequence
  Non-phosphorylated peptide with phosphomimetic D/E amino acids, all E
EVAYGYDHEPPGKE

SEQ ID NO: 12 - protein, 14 aa, Artificial Sequence
  Non-phosphorylated peptide with phosphomimetic D/E amino acids, all D
DVAYGYDHDPPGKD

SEQ ID NO: 13 - protein, 14 aa, Artificial Sequence
  Non-phosphorylated peptide with phosphomimetic D/E amino acids
EVAYGYDHDPPGKE

SEQ ID NO: 14 - protein, 14 aa, Artificial Sequence
  Non-phosphorylated peptide with phosphomimetic D/E amino acids
EVAYGYDHEPPGKD

SEQ ID NO: 15 - protein, 14 aa, Artificial Sequence
  Non-phosphorylated peptide with phosphomimetic D/E amino acids
EVAYGYDHDPPGKD

SEQ ID NO: 16 - protein, 14 aa, Artificial Sequence
  Non-phosphorylated peptide with phosphomimetic D/E amino acids
DVAYGYDHEPPGKE

SEQ ID NO: 17 - protein, 14 aa, Artificial Sequence
  Non-phosphorylated peptide with phosphomimetic D/E amino acids
DVAYGYDHDPPGKE

SEQ ID NO: 18 - protein, 14 aa, Artificial Sequence
  Non-phosphorylated peptide with phosphomimetic D/E amino acids
DVAYGYDHEPPGKD

SEQ ID NO: 19 - protein, 14 aa, Artificial Sequence
  Synthetic Sequence
  MISC_FEATURE (1)..(1): Xaa is serine (S), threonine (T), aspartic acid (D), glutamic acid (E), 2-amino-4-phosphobutyric acid (L-AP4, L-APB), or thiophosphoserine
  MISC_FEATURE (4)..(4): Xaa is  tyrosine (Y) or and 4-phosphomethyl- L-phenylalanine (Pmp)
  MISC_FEATURE (6)..(6): Xaa is  tyrosine (Y) or and 4-phosphomethyl- L-phenylalanine (Pmp)
  MISC_FEATURE (9)..(9): Xaa is serine (S), threonine (T), aspartic acid (D), glutamic acid (E), 2-amino-4-phosphobutyric acid (L-AP4, L-APB), or thiophosphoserine
  MISC_FEATURE (14)..(14): Xaa is serine (S), threonine (T), aspartic acid (D), glutamic acid (E), 2-amino-4-phosphobutyric acid (L-AP4, L-APB), or thiophosphoserine
XVAXGXDHXPPGKX

SEQ ID NO: 20 - protein, 4 aa, Artificial Sequence
  can be appended to the N-terminus of SEQ ID NO19
DEEL

SEQ ID NO: 21 - protein, 5 aa, Artificial Sequence
  can be appended to the N-terminus of SEQ ID NO19
ADEEL

SEQ ID NO: 22 - protein, 4 aa, Artificial Sequence
  can be appended to the C-terminus of SEQ ID NO19
GPEA

SEQ ID NO: 23 - protein, 5 aa, Artificial Sequence
  can be appended to the N-terminus of SEQ ID NO19
GPEAP

SEQ ID NO: 24 - protein, 24 aa, Artificial Sequence
  Extended phosphomimetic peptide EEE
ADEELEVAYGYDHEPPGKEGPEAP

SEQ ID NO: 25 - protein, 24 aa, Artificial Sequence
  Extended phosphomimetic peptide EDE
ADEELEVAYGYDHDPPGKEGPEAP

SEQ ID NO: 26 - protein, 24 aa, Artificial Sequence
  Extended phosphomimetic peptide EED
ADEELEVAYGYDHEPPGKDGPEAP

SEQ ID NO: 27 - protein, 24 aa, Artificial Sequence
  Extended phosphomimetic peptide EDD
ADEELEVAYGYDHDPPGKDGPEAP

SEQ ID NO: 28 - protein, 24 aa, Artificial Sequence
  Extended phosphomimetic peptide DEE
ADEELDVAYGYDHEPPGKEGPEAP

SEQ ID NO: 29 - protein, 24 aa, Artificial Sequence
  Extended phosphomimetic peptide DED
ADEELDVAYGYDHEPPGKDGPEAP

SEQ ID NO: 30 - protein, 24 aa, Artificial Sequence
  Extended phosphomimetic peptide DDE
ADEELDVAYGYDHDPPGKEGPEAP

SEQ ID NO: 31 - protein, 24 aa, Artificial Sequence
  Extended phosphomimetic peptide DDD
ADEELDVAYGYDHDPPGKDGPEAP

SEQ ID NO: 32 - protein, 24 aa, Artificial Sequence
  Extended phosphomimetic peptide
ADEELTVAYGYDHSPPGKSGPEAP

SEQ ID NO: 33 - DNA, 75 bp, Artificial Sequence
  Synthetic Sequence
  misc_feature (3)..(3): N is any nucleotide (e.g., A, G, T, U, or C)
  misc_feature (5)..(5): N is a pyrimidine (e.g., C, T, or U)
  misc_feature (6)..(6): n is a, c, g, t or u
  misc_feature (9)..(9): N is a purine (e.g., A or G)
  misc_feature (11)..(11): N is a purine (e.g., A or G)
  misc_feature (13)..(13): N is a pyrimidine (e.g., C, T, or U)
  misc_feature (15)..(15): N is any nucleotide (e.g., A, G, T, U, or C)
  misc_feature (18)..(18): N is a purine (e.g., A or G)
  misc_feature (21)..(21): N is any nucleotide (e.g., A, G, T, U, or C)
  misc_feature (24)..(24): N is any nucleotide (e.g., A, G, T, U, or C)
  misc_feature (27)..(27): N is a pyrimidine (e.g., C, T, or U)
  misc_feature (30)..(30): N is any nucleotide (e.g., A, G, T, U, or C)
  misc_feature (33)..(33): N is a pyrimidine (e.g., C, T, or U)
  misc_feature (36)..(36): N is a pyrimidine (e.g., C, T, or U)
  misc_feature (39)..(39): N is a pyrimidine (e.g., C, T, or U)
  misc_feature (42)..(42): N is a pyrimidine (e.g., C, T, or U)
  misc_feature (45)..(45): N is any nucleotide (e.g., A, G, T, U, or C)
  misc_feature (48)..(48): N is any nucleotide (e.g., A, G, T, U, or C)
  misc_feature (51)..(51): N is any nucleotide (e.g., A, G, T, U, or C)
  misc_feature (54)..(54): N is a purine (e.g., A or G)
  misc_feature (57)..(57): N is a pyrimidine (e.g., C, T, or U)
  misc_feature (60)..(60): N is any nucleotide (e.g., A, G, T, U, or C)
  misc_feature (63)..(63): N is any nucleotide (e.g., A, G, T, U, or C)
  misc_feature (66)..(66): N is a purine (e.g., A or G)
  misc_feature (69)..(69): N is any nucleotide (e.g., A, G, T, U, or C)
  misc_feature (72)..(72): N is any nucleotide (e.g., A, G, T, U, or C)
  misc_feature (74)..(74): N is a purine (e.g., A or G)
  misc_feature (75)..(75): N is a purine (e.g., A or G)
gcngangargarnungargungcnuanggnuangancanganccnccnggnaarganggnccngargcnccnurr

SEQ ID NO: 34 - DNA, 75 bp, Artificial Sequence
  Synthetic Sequence
  misc_feature (3)..(3): N is any nucleotide (e.g., A, G, T, U, or C)
  misc_feature (6)..(6): N is a pyrimidine (e.g., C, T, or U)
  misc_feature (9)..(9): N is a purine (A or G)
  misc_feature (11)..(11): N  is a purine (A or G)
  misc_feature (12)..(12): n is a, c, g, t or u
  misc_feature (13)..(13): N is a pyrimidine (e.g., C, T, or U)
  misc_feature (15)..(15): N is any nucleotide (e.g., A, G, T, U, or C)
  misc_feature (18)..(18): N is any nucleotide (e.g., A, G, T, U, or C)
  misc_feature (21)..(21): N is any nucleotide (e.g., A, G, T, U, or C)
  misc_feature (24)..(24): N is any nucleotide (e.g., A, G, T, U, or C)
  misc_feature (27)..(27): N is a pyrimidine (e.g., C, T, or U)
  misc_feature (30)..(30): N is any nucleotide (e.g., A, G, T, U, or C)
  misc_feature (33)..(33): N is a pyrimidine (e.g., C, T, or U)
  misc_feature (36)..(36): N is a pyrimidine (e.g., C, T, or U)
  misc_feature (39)..(39): N is a pyrimidine (e.g., C, T, or U)
  misc_feature (40)..(40): N  is a weak bonding nucleotide (e.g., A, T or U)
  misc_feature (41)..(41): N  is a strong bonding nucleotide (e.g., G or C)
  misc_feature (42)..(42): N is any nucleotide (e.g., A, G, T, U, or C)
  misc_feature (45)..(45): N is any nucleotide (e.g., A, G, T, U, or C)
  misc_feature (48)..(48): N is any nucleotide (e.g., A, G, T, U, or C)
  misc_feature (51)..(51): N is any nucleotide (e.g., A, G, T, U, or C)
  misc_feature (54)..(54): N  is a purine (A or G)
  misc_feature (55)..(55): N  is a weak bonding nucleotide (e.g., A, T or U)
  misc_feature (56)..(56): N  is a strong bonding nucleotide (e.g., G or C)
  misc_feature (57)..(57): N is any nucleotide (e.g., A, G, T, U, or C)
  misc_feature (60)..(60): N is any nucleotide (e.g., A, G, T, U, or C)
  misc_feature (63)..(63): N is any nucleotide (e.g., A, G, T, U, or C)
  misc_feature (66)..(66): N is a purine (A or G)
  misc_feature (69)..(69): N is any nucleotide (e.g., A, G, T, U, or C)
  misc_feature (72)..(72): N is any nucleotide (e.g., A, G, T, U, or C)
  misc_feature (74)..(74): N is a purine (A or G)
  misc_feature (75)..(75): N is a purine (A or G)
gcngangangannunacngungcnuanggnuangancannnnccnccnggnaannnnggnccngangcnccnunn

SEQ ID NO: 35 - protein, 366 aa, Artificial Sequence
  Synthetic Sequence
  misc_feature (332)..(332): Xaa can be any naturally occurring amino acid
  misc_feature (340)..(340): Xaa can be any naturally occurring amino acid
  misc_feature (345)..(345): Xaa can be any naturally occurring amino acid
MDSDDEMVEEAVEGHLDDDGLPHGFCTVTYSSTDRFEGNFVHGEKNGRGKFFFFDGSTLEGYYVDDALQGQGVYTYEDGGVLQGTYVDGELNGPAQEYDTDGRLIFKGQYKDNIRHGVCWIYYPDGGSLVGEVNEDGEMTGEKIAYVYPDERTALYGKFIDGEMIEGKLATLMSTEEGRPHFELMPGNSVYHFDKSTSSCISTNALLPDPYESERVYVAESLISSAGEGLFSKVAVGPNTVMSFYNGVRITHQEVDSRDWALNGNTLSLDEETVIDVPEPYNHVSKYCASLGHKANASFTPNCIYDMFVHPRFGPIKCIRTLRAVEADEELXVAYGYDHXPPGKXGPEAPEWYQVELKAFQATQQK

SEQ ID NO: 36 - protein, 961 aa, Artificial Sequence
  Synthetic Sequence
AGGAYWSNGAYGAYGARAGGNGARGARGCNGNGARGGNCAYYNGAYGAYGAYGGNYNCCNCAYGGNYGYACNGNACNAYWSNWSNACNGAYMGNYGARGGNAAYYGNCAYGGNGARAARAAYGGNMGNGGNAARYYYYGAYGGNWSNACNYNGARGGNAYAYGNGAYGAYGCNYNCARGGNCARGGNGNAYACNAYGARGAYGGNGGNGNYNCARGGNACNAYGNGAYGGNGARYNAAYGGNCCNGCNCARGARAYGAYACNGAYGGNMGNYNAHYAARGGNCARAYAARGAYAAYAHMGNCAYGGNGNGYGGAHAYAYCCNGAYGGNGGNWSNYNGNGGNGARGNAAYGARGAYGGNGARAGACNGGNGARAARAHGCNAYGNAYCCNGAYGARMGNACNGCNYNAYGGNAARYAHGAYGGNGARAGAHGARGGNAARYNGCNACNYNAGWSNACNGARGARGGNMGNCCNCAYYGARYNAGCCNGGNAAYWSNGNAYCAYYGAYAARWSNACNWSNWSNGYAHWSNACNAAYGCNYNYNCCNGAYCCNAYGARWSNGARMGNGNAYGNGCNGARWSNYNAHWSNWSNGCNGGNGARGGNYNYWSNAARGNGCNGNGGNCCNAAYACNGNAGWSNYAYAAYGGNGNMGNAHACNCAYCARGARGNGAYWSNMGNGAYGGGCNYNAAYGGNAAYACNYNWSNYNGAYGARGARACNGNAHGAYGNCCNGARCCNAYAAYCAYGNWSNAARAYGYGCNWSNYNGGNCAYAARGCNAAYGCNWSNYACNCCNAAYGYAHAYGAYAGYGNCAYCCNMGNYGGNCCNAHAARGYAHMGNACNYNMGNGCNGNGARGCNGAYGARGARYNGARGNGCNAYGGNAYGAYCAYGAYCCNCCNGGNAARGAYGGNCCNGARGCNCCNGARGGAYCARGNGARYNAARGCNYCARGCNACNCARCARAAR

SEQ ID NO: 37 - protein, 366 aa, Artificial Sequence
  Synthetic Sequence
MDSDDEMVEEAVEGHLDDDGLPHGFCTVTYSSTDRFEGNFVHGEKNGRGKFFFFDGSTLEGYYVDDALQGQGVYTYEDGGVLQGTYVDGELNGPAQEYDTDGRLIFKGQYKDNIRHGVCWIYYPDGGSLVGEVNEDGEMTGEKIAYVYPDERTALYGKFIDGEMIEGKLATLMSTEEGRPHFELMPGNSVYHFDKSTSSCISTNALLPDPYESERVYVAESLISSAGEGLFSKVAVGPNTVMSFYNGVRITHQEVDSRDWALNGNTLSLDEETVIDVPEPYNHVSKYCASLGHKANHSFTPNCIYDMFVHPRFGPIKCIRTLRAVEADEELTVAYGYDHSPPGKSGPEAPEWYQVELKAFQATQQK

SEQ ID NO: 38 - protein, 24 aa, Artificial Sequence
  Phospho-mimicking peptide sequence
  MISC_FEATURE (1)..(24): Xaa is either Asp or Glu
ADEELXVAYGYDHXPPGKXGPEAP

SEQ ID NO: 39 - DNA, 1101 bp, Artificial Sequence
  Synthetic Sequence
atggatagcgacgacgagatggtggaggaggcggtggaagggcacctggacgatgacggattaccgcacgggttctgcacagtcacctactcctccacagacagatttgaggggaactttgttcacggagaaaagaacggacgggggaagttcttcttctttgatggcagcaccctggaggggtattatgtggatgatgccttgcagggccagggagtttacacttacgaagatgggggagttctccagggcacgtatgtagacggagagctgaacggtccagcccaggaatatgacacagatgggagactgatcttcaaggggcagtataaagataacattcgtcatggagtgtgctggatatattacccagatggaggaagccttgtaggagaagtaaatgaagatggggagatgactggagagaagatagcctatgtgtaccctgatgagaggaccgcactttatgggaaatttattgatggagagatgatagaaggcaaactggctacccttatgtccactgaagaagggaggcctcactttgaactgatgcctggaaattcagtgtaccactttgataagtcgacttcatcttgcatttctaccaatgctcttcttccagatccttatgaatcagaaagggtttatgttgctgaatctcttatttccagtgctggagaaggacttttttcaaaggtagctgtgggacctaatactgttatgtctttttataatggagttcgaattacacaccaagaggttgacagcagggactgggcccttaatgggaacaccctctcccttgatgaagaaacggtcattgatgtgcctgagccctataaccacgtatccaagtactgtgcctccttgggacacaaggcaaatcactccttcactccaaactgcatctacgatatgtttgtccacccccgttttgggcccatcaaatgcatccgcaccctgagagcagtggaggccgatgaagagctcaccgttgcctatggctatgaccacagcccccccgggaagagtgggcctgaagcccctgagtggtaccaggtggagctgaaggccttccaggccacccagcaaaagtga